SEQUENCE LISTING (575 sequences)

SEQ ID NO: 1 - protein, 335 aa, Mus musculus
MCPQKLTISWFAIVLLVSPLMAMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRS

SEQ ID NO: 2 - protein, 162 aa, Homo sapiens
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 3 - protein, 267 aa, Homo sapiens
MAPRRARGCRTLGLPALLLLLLLRPPATRGITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 4 - protein, 234 aa, Homo sapiens
MAPRRARGCRTLGLPALLLLLLLRPPATRGITCPPPMSVEHADIWVKSYSLYSRERYICNSGFKRKAGTSSLTECVLNKATNVAHWTTPSLKCIKPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 5 - protein, 231 aa, Homo sapiens
MSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 6 - protein, 353 aa, Homo sapiens
MRLAGRQVPEQRSPPPPGLGSARPGSPAVSCGAAAMAPRRARGCRTLGLPALLLLLLLRPPATRDARDRLAVLAGRSRISESFNHEVQTHEACVRLRTMENCPQCHHHRTSRQQAGITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 7 - protein, 7 aa, synthetic construct
  REGION 1..7: note = Synthetic sequence
  REGION 1..7: note = misc_feature - Synthetic sequence
KVSALKE

SEQ ID NO: 8 - protein, 7 aa, synthetic construct
  REGION 1..7: note = Synthetic sequence
EVSALEK

SEQ ID NO: 9 - protein, 35 aa, synthetic construct
  REGION 1..35: note = Synthetic sequence
KVSALKEKVSALKEKVSALKEKVSALKEKVSALKE

SEQ ID NO: 10 - protein, 35 aa, synthetic construct
  REGION 1..35: note = Synthetic sequence
EVSALEKEVSALEKEVSALEKEVSALEKEVSALEK

SEQ ID NO: 11 - protein, 96 aa, Homo sapiens
MAPRRARGCRTLGLPALLLLLLLRPPATRGITCPPPMSVEHADIWVKSYSLYSRERYICNSGFKRKAGTSSLTECVLNKATNVAHWTTPSLKCIRD

SEQ ID NO: 12 - protein, 146 aa, Homo sapiens
MAPRRARGCRTLGLPALLLLLLLRPPATRGITCPPPMSVEHADIWVKSYSLYSRERYICNSGFKRKAGTSSLTECVLNKATNVAHWTTPSLKCIRDGGGGSGGGGSGGGGSEVSALEKEVSALEKEVSALEKEVSALEKEVSALEK

SEQ ID NO: 13 - protein, 403 aa, Homo sapiens
MRLAGRQVPEQRSPPPPGLGSARPGSPAVSCGAAAMAPRRARGCRTLGLPALLLLLLLRPPATRDARDRLAVLAGRSRISESFNHEVQTHEACVRLRTMENCPQCHHHRTSRQQAGITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHLGGGGSGGGGSGGGGSEVSALEKEVSALEKEVSALEKEVSALEKEVSALEK

SEQ ID NO: 14 - protein, 317 aa, Homo sapiens
MAPRRARGCRTLGLPALLLLLLLRPPATRGITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHLGGGGSGGGGSGGGGSEVSALEKEVSALEKEVSALEKEVSALEKEVSALEK

SEQ ID NO: 15 - protein, 215 aa, Homo sapiens
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGSGGGGSGGGGSCGGKVSALKEKVSALKEKVSALKEKVSALKEKVSALKE

SEQ ID NO: 16 - protein, 14 aa, Homo sapiens
TAHPSPSPRSAGQF

SEQ ID NO: 17 - protein, 10 aa, Homo sapiens
EYRMSPSNQT

SEQ ID NO: 18 - protein, 10 aa, Homo sapiens
YYRMSPSNQT

SEQ ID NO: 19 - protein, 14 aa, Mus musculus
TRYPSPSPKPEGRF

SEQ ID NO: 20 - protein, 10 aa, Homo sapiens
WNRLSPSNQT

SEQ ID NO: 21 - protein, 14 aa, Homo sapiens
TAHPSPSPRSAGQA

SEQ ID NO: 22 - protein, 14 aa, Homo sapiens
TAHPSPSPRSAGQR

SEQ ID NO: 23 - protein, 14 aa, Homo sapiens
TAHPSPSPRSAGQN

SEQ ID NO: 24 - protein, 14 aa, Homo sapiens
TAHPSPSPRSAGQD

SEQ ID NO: 25 - protein, 14 aa, Homo sapiens
TAHPSPSPRSAGQC

SEQ ID NO: 26 - protein, 14 aa, Homo sapiens
TAHPSPSPRSAGQQ

SEQ ID NO: 27 - protein, 14 aa, Homo sapiens
TAHPSPSPRSAGQE

SEQ ID NO: 28 - protein, 14 aa, Homo sapiens
TAHPSPSPRSAGQG

SEQ ID NO: 29 - protein, 14 aa, Homo sapiens
TAHPSPSPRSAGQH

SEQ ID NO: 30 - protein, 14 aa, Homo sapiens
TAHPSPSPRSAGQI

SEQ ID NO: 31 - protein, 14 aa, Homo sapiens
TAHPSPSPRSAGQL

SEQ ID NO: 32 - protein, 14 aa, Homo sapiens
TAHPSPSPRSAGQK

SEQ ID NO: 33 - protein, 14 aa, Homo sapiens
TAHPSPSPRSAGQM

SEQ ID NO: 34 - protein, 14 aa, Homo sapiens
TAHPSPSPRSAGQF

SEQ ID NO: 35 - protein, 14 aa, Homo sapiens
TAHPSPSPRSAGQP

SEQ ID NO: 36 - protein, 14 aa, Homo sapiens
TAHPSPSPRSAGQS

SEQ ID NO: 37 - protein, 14 aa, Homo sapiens
TAHPSPSPRSAGQT

SEQ ID NO: 38 - protein, 14 aa, Homo sapiens
TAHPSPSPRSAGQW

SEQ ID NO: 39 - protein, 14 aa, Homo sapiens
TAHPSPSPRSAGQY

SEQ ID NO: 40 - protein, 14 aa, Homo sapiens
TAHPSPSPRSAGQV

SEQ ID NO: 41 - protein, 14 aa, Homo sapiens
TAHPSPSPRSAGAF

SEQ ID NO: 42 - protein, 14 aa, Homo sapiens
TAHPSPSPRSAGRF

SEQ ID NO: 43 - protein, 14 aa, Homo sapiens
TAHPSPSPRSAGNF

SEQ ID NO: 44 - protein, 14 aa, Homo sapiens
TAHPSPSPRSAGDF

SEQ ID NO: 45 - protein, 14 aa, Homo sapiens
TAHPSPSPRSAGCF

SEQ ID NO: 46 - protein, 14 aa, Homo sapiens
TAHPSPSPRSAGQF

SEQ ID NO: 47 - protein, 14 aa, Homo sapiens
TAHPSPSPRSAGEF

SEQ ID NO: 48 - protein, 14 aa, Homo sapiens
TAHPSPSPRSAGGF

SEQ ID NO: 49 - protein, 14 aa, Homo sapiens
TAHPSPSPRSAGHF

SEQ ID NO: 50 - protein, 14 aa, Homo sapiens
TAHPSPSPRSAGIF

SEQ ID NO: 51 - protein, 14 aa, Homo sapiens
TAHPSPSPRSAGLF

SEQ ID NO: 52 - protein, 14 aa, Homo sapiens
TAHPSPSPRSAGKF

SEQ ID NO: 53 - protein, 14 aa, Homo sapiens
TAHPSPSPRSAGMF

SEQ ID NO: 54 - protein, 14 aa, Homo sapiens
TAHPSPSPRSAGFF

SEQ ID NO: 55 - protein, 14 aa, Homo sapiens
TAHPSPSPRSAGPF

SEQ ID NO: 56 - protein, 14 aa, Homo sapiens
TAHPSPSPRSAGSF

SEQ ID NO: 57 - protein, 14 aa, Homo sapiens
TAHPSPSPRSAGTF

SEQ ID NO: 58 - protein, 14 aa, Homo sapiens
TAHPSPSPRSAGWF

SEQ ID NO: 59 - protein, 14 aa, Homo sapiens
TAHPSPSPRSAGYF

SEQ ID NO: 60 - protein, 14 aa, Homo sapiens
TAHPSPSPRSAGVF

SEQ ID NO: 61 - protein, 14 aa, Homo sapiens
TAHPSPSPRSAAQF

SEQ ID NO: 62 - protein, 14 aa, Homo sapiens
TAHPSPSPRSARQF

SEQ ID NO: 63 - protein, 14 aa, Homo sapiens
TAHPSPSPRSANQF

SEQ ID NO: 64 - protein, 14 aa, Homo sapiens
TAHPSPSPRSADQF

SEQ ID NO: 65 - protein, 14 aa, Homo sapiens
TAHPSPSPRSACQF

SEQ ID NO: 66 - protein, 14 aa, Homo sapiens
TAHPSPSPRSAQQF

SEQ ID NO: 67 - protein, 14 aa, Homo sapiens
TAHPSPSPRSAEQF

SEQ ID NO: 68 - protein, 14 aa, Homo sapiens
TAHPSPSPRSAGQF

SEQ ID NO: 69 - protein, 14 aa, Homo sapiens
TAHPSPSPRSAHQF

SEQ ID NO: 70 - protein, 14 aa, Homo sapiens
TAHPSPSPRSAIQF

SEQ ID NO: 71 - protein, 14 aa, Homo sapiens
TAHPSPSPRSALQF

SEQ ID NO: 72 - protein, 14 aa, Homo sapiens
TAHPSPSPRSAKQF

SEQ ID NO: 73 - protein, 14 aa, Homo sapiens
TAHPSPSPRSAMQF

SEQ ID NO: 74 - protein, 14 aa, Homo sapiens
TAHPSPSPRSAFQF

SEQ ID NO: 75 - protein, 14 aa, Homo sapiens
TAHPSPSPRSAPQF

SEQ ID NO: 76 - protein, 14 aa, Homo sapiens
TAHPSPSPRSASQF

SEQ ID NO: 77 - protein, 14 aa, Homo sapiens
TAHPSPSPRSATQF

SEQ ID NO: 78 - protein, 14 aa, Homo sapiens
TAHPSPSPRSAWQF

SEQ ID NO: 79 - protein, 14 aa, Homo sapiens
TAHPSPSPRSAYQF

SEQ ID NO: 80 - protein, 14 aa, Homo sapiens
TAHPSPSPRSAVQF

SEQ ID NO: 81 - protein, 14 aa, Homo sapiens
TAHPSPSPRSAGQF

SEQ ID NO: 82 - protein, 14 aa, Homo sapiens
TAHPSPSPRSRGQF

SEQ ID NO: 83 - protein, 14 aa, Homo sapiens
TAHPSPSPRSNGQF

SEQ ID NO: 84 - protein, 14 aa, Homo sapiens
TAHPSPSPRSDGQF

SEQ ID NO: 85 - protein, 14 aa, Homo sapiens
TAHPSPSPRSCGQF

SEQ ID NO: 86 - protein, 14 aa, Homo sapiens
TAHPSPSPRSQGQF

SEQ ID NO: 87 - protein, 14 aa, Homo sapiens
TAHPSPSPRSEGQF

SEQ ID NO: 88 - protein, 14 aa, Homo sapiens
TAHPSPSPRSGGQF

SEQ ID NO: 89 - protein, 14 aa, Homo sapiens
TAHPSPSPRSHGQF

SEQ ID NO: 90 - protein, 14 aa, Homo sapiens
TAHPSPSPRSIGQF

SEQ ID NO: 91 - protein, 14 aa, Homo sapiens
TAHPSPSPRSLGQF

SEQ ID NO: 92 - protein, 14 aa, Homo sapiens
TAHPSPSPRSKGQF

SEQ ID NO: 93 - protein, 14 aa, Homo sapiens
TAHPSPSPRSMGQF

SEQ ID NO: 94 - protein, 14 aa, Homo sapiens
TAHPSPSPRSFGQF

SEQ ID NO: 95 - protein, 14 aa, Homo sapiens
TAHPSPSPRSPGQF

SEQ ID NO: 96 - protein, 14 aa, Homo sapiens
TAHPSPSPRSSGQF

SEQ ID NO: 97 - protein, 14 aa, Homo sapiens
TAHPSPSPRSTGQF

SEQ ID NO: 98 - protein, 14 aa, Homo sapiens
TAHPSPSPRSWGQF

SEQ ID NO: 99 - protein, 14 aa, Homo sapiens
TAHPSPSPRSYGQF

SEQ ID NO: 100 - protein, 14 aa, Homo sapiens
TAHPSPSPRSVGQF

SEQ ID NO: 101 - protein, 14 aa, Homo sapiens
TAHPSPSPRAAGQF

SEQ ID NO: 102 - protein, 14 aa, Homo sapiens
TAHPSPSPRRAGQF

SEQ ID NO: 103 - protein, 14 aa, Homo sapiens
TAHPSPSPRNAGQF

SEQ ID NO: 104 - protein, 14 aa, Homo sapiens
TAHPSPSPRDAGQF

SEQ ID NO: 105 - protein, 14 aa, Homo sapiens
TAHPSPSPRCAGQF

SEQ ID NO: 106 - protein, 14 aa, Homo sapiens
TAHPSPSPRQAGQF

SEQ ID NO: 107 - protein, 14 aa, Homo sapiens
TAHPSPSPREAGQF

SEQ ID NO: 108 - protein, 14 aa, Homo sapiens
TAHPSPSPRGAGQF

SEQ ID NO: 109 - protein, 14 aa, Homo sapiens
TAHPSPSPRHAGQF

SEQ ID NO: 110 - protein, 14 aa, Homo sapiens
TAHPSPSPRIAGQF

SEQ ID NO: 111 - protein, 14 aa, Homo sapiens
TAHPSPSPRLAGQF

SEQ ID NO: 112 - protein, 14 aa, Homo sapiens
TAHPSPSPRKAGQF

SEQ ID NO: 113 - protein, 14 aa, Homo sapiens
TAHPSPSPRMAGQF

SEQ ID NO: 114 - protein, 14 aa, Homo sapiens
TAHPSPSPRFAGQF

SEQ ID NO: 115 - protein, 14 aa, Homo sapiens
TAHPSPSPRPAGQF

SEQ ID NO: 116 - protein, 14 aa, Homo sapiens
TAHPSPSPRSAGQF

SEQ ID NO: 117 - protein, 14 aa, Homo sapiens
TAHPSPSPRTAGQF

SEQ ID NO: 118 - protein, 14 aa, Homo sapiens
TAHPSPSPRWAGQF

SEQ ID NO: 119 - protein, 14 aa, Homo sapiens
TAHPSPSPRYAGQF

SEQ ID NO: 120 - protein, 14 aa, Homo sapiens
TAHPSPSPRVAGQF

SEQ ID NO: 121 - protein, 14 aa, Homo sapiens
TAHPSPSPASAGQF

SEQ ID NO: 122 - protein, 14 aa, Homo sapiens
TAHPSPSPRSAGQF

SEQ ID NO: 123 - protein, 14 aa, Homo sapiens
TAHPSPSPNSAGQF

SEQ ID NO: 124 - protein, 14 aa, Homo sapiens
TAHPSPSPDSAGQF

SEQ ID NO: 125 - protein, 14 aa, Homo sapiens
TAHPSPSPCSAGQF

SEQ ID NO: 126 - protein, 14 aa, Homo sapiens
TAHPSPSPQSAGQF

SEQ ID NO: 127 - protein, 14 aa, Homo sapiens
TAHPSPSPESAGQF

SEQ ID NO: 128 - protein, 14 aa, Homo sapiens
TAHPSPSPGSAGQF

SEQ ID NO: 129 - protein, 14 aa, Homo sapiens
TAHPSPSPHSAGQF

SEQ ID NO: 130 - protein, 14 aa, Homo sapiens
TAHPSPSPISAGQF

SEQ ID NO: 131 - protein, 14 aa, Homo sapiens
TAHPSPSPLSAGQF

SEQ ID NO: 132 - protein, 14 aa, Homo sapiens
TAHPSPSPKSAGQF

SEQ ID NO: 133 - protein, 14 aa, Homo sapiens
TAHPSPSPMSAGQF

SEQ ID NO: 134 - protein, 14 aa, Homo sapiens
TAHPSPSPFSAGQF

SEQ ID NO: 135 - protein, 14 aa, Homo sapiens
TAHPSPSPPSAGQF

SEQ ID NO: 136 - protein, 14 aa, Homo sapiens
TAHPSPSPSSAGQF

SEQ ID NO: 137 - protein, 14 aa, Homo sapiens
TAHPSPSPTSAGQF

SEQ ID NO: 138 - protein, 14 aa, Homo sapiens
TAHPSPSPWSAGQF

SEQ ID NO: 139 - protein, 14 aa, Homo sapiens
TAHPSPSPYSAGQF

SEQ ID NO: 140 - protein, 14 aa, Homo sapiens
TAHPSPSPVSAGQF

SEQ ID NO: 141 - protein, 14 aa, Homo sapiens
TAHPSPSARSAGQF

SEQ ID NO: 142 - protein, 14 aa, Homo sapiens
TAHPSPSRRSAGQF

SEQ ID NO: 143 - protein, 14 aa, Homo sapiens
TAHPSPSNRSAGQF

SEQ ID NO: 144 - protein, 14 aa, Homo sapiens
TAHPSPSDRSAGQF

SEQ ID NO: 145 - protein, 14 aa, Homo sapiens
TAHPSPSCRSAGQF

SEQ ID NO: 146 - protein, 14 aa, Homo sapiens
TAHPSPSQRSAGQF

SEQ ID NO: 147 - protein, 14 aa, Homo sapiens
TAHPSPSERSAGQF

SEQ ID NO: 148 - protein, 14 aa, Homo sapiens
TAHPSPSGRSAGQF

SEQ ID NO: 149 - protein, 14 aa, Homo sapiens
TAHPSPSHRSAGQF

SEQ ID NO: 150 - protein, 14 aa, Homo sapiens
TAHPSPSIRSAGQF

SEQ ID NO: 151 - protein, 14 aa, Homo sapiens
TAHPSPSLRSAGQF

SEQ ID NO: 152 - protein, 14 aa, Homo sapiens
TAHPSPSKRSAGQF

SEQ ID NO: 153 - protein, 14 aa, Homo sapiens
TAHPSPSMRSAGQF

SEQ ID NO: 154 - protein, 14 aa, Homo sapiens
TAHPSPSFRSAGQF

SEQ ID NO: 155 - protein, 14 aa, Homo sapiens
TAHPSPSPRSAGQF

SEQ ID NO: 156 - protein, 14 aa, Homo sapiens
TAHPSPSSRSAGQF

SEQ ID NO: 157 - protein, 14 aa, Homo sapiens
TAHPSPSTRSAGQF

SEQ ID NO: 158 - protein, 14 aa, Homo sapiens
TAHPSPSWRSAGQF

SEQ ID NO: 159 - protein, 14 aa, Homo sapiens
TAHPSPSYRSAGQF

SEQ ID NO: 160 - protein, 14 aa, Homo sapiens
TAHPSPSVRSAGQF

SEQ ID NO: 161 - protein, 14 aa, Homo sapiens
TAHPSPAPRSAGQF

SEQ ID NO: 162 - protein, 14 aa, Homo sapiens
TAHPSPRPRSAGQF

SEQ ID NO: 163 - protein, 14 aa, Homo sapiens
TAHPSPNPRSAGQF

SEQ ID NO: 164 - protein, 14 aa, Homo sapiens
TAHPSPDPRSAGQF

SEQ ID NO: 165 - protein, 14 aa, Homo sapiens
TAHPSPCPRSAGQF

SEQ ID NO: 166 - protein, 14 aa, Homo sapiens
TAHPSPEPRSAGQF

SEQ ID NO: 167 - protein, 14 aa, Homo sapiens
TAHPSPQPRSAGQF

SEQ ID NO: 168 - protein, 14 aa, Homo sapiens
TAHPSPGPRSAGQF

SEQ ID NO: 169 - protein, 14 aa, Homo sapiens
TAHPSPHPRSAGQF

SEQ ID NO: 170 - protein, 14 aa, Homo sapiens
TAHPSPIPRSAGQF

SEQ ID NO: 171 - protein, 14 aa, Homo sapiens
TAHPSPLPRSAGQF

SEQ ID NO: 172 - protein, 14 aa, Homo sapiens
TAHPSPKPRSAGQF

SEQ ID NO: 173 - protein, 14 aa, Homo sapiens
TAHPSPMPRSAGQF

SEQ ID NO: 174 - protein, 14 aa, Homo sapiens
TAHPSPFPRSAGQF

SEQ ID NO: 175 - protein, 14 aa, Homo sapiens
TAHPSPPPRSAGQF

SEQ ID NO: 176 - protein, 14 aa, Homo sapiens
TAHPSPSPRSAGQF

SEQ ID NO: 177 - protein, 14 aa, Homo sapiens
TAHPSPTPRSAGQF

SEQ ID NO: 178 - protein, 14 aa, Homo sapiens
TAHPSPWPRSAGQF

SEQ ID NO: 179 - protein, 14 aa, Homo sapiens
TAHPSPYPRSAGQF

SEQ ID NO: 180 - protein, 14 aa, Homo sapiens
TAHPSPVPRSAGQF

SEQ ID NO: 181 - protein, 14 aa, Homo sapiens
TAHPSASPRSAGQF

SEQ ID NO: 182 - protein, 14 aa, Homo sapiens
TAHPSRSPRSAGQF

SEQ ID NO: 183 - protein, 14 aa, Homo sapiens
TAHPSNSPRSAGQF

SEQ ID NO: 184 - protein, 14 aa, Homo sapiens
TAHPSDSPRSAGQF

SEQ ID NO: 185 - protein, 14 aa, Homo sapiens
TAHPSCSPRSAGQF

SEQ ID NO: 186 - protein, 14 aa, Homo sapiens
TAHPSQSPRSAGQF

SEQ ID NO: 187 - protein, 14 aa, Homo sapiens
TAHPSESPRSAGQF

SEQ ID NO: 188 - protein, 14 aa, Homo sapiens
TAHPSGSPRSAGQF

SEQ ID NO: 189 - protein, 14 aa, Homo sapiens
TAHPSHSPRSAGQF

SEQ ID NO: 190 - protein, 14 aa, Homo sapiens
TAHPSISPRSAGQF

SEQ ID NO: 191 - protein, 14 aa, Homo sapiens
TAHPSLSPRSAGQF

SEQ ID NO: 192 - protein, 14 aa, Homo sapiens
TAHPSKSPRSAGQF

SEQ ID NO: 193 - protein, 14 aa, Homo sapiens
TAHPSMSPRSAGQF

SEQ ID NO: 194 - protein, 14 aa, Homo sapiens
TAHPSFSPRSAGQF

SEQ ID NO: 195 - protein, 14 aa, Homo sapiens
TAHPSPSPRSAGQF

SEQ ID NO: 196 - protein, 14 aa, Homo sapiens
TAHPSSSPRSAGQF

SEQ ID NO: 197 - protein, 14 aa, Homo sapiens
TAHPSTSPRSAGQF

SEQ ID NO: 198 - protein, 14 aa, Homo sapiens
TAHPSWSPRSAGQF

SEQ ID NO: 199 - protein, 14 aa, Homo sapiens
TAHPSYSPRSAGQF

SEQ ID NO: 200 - protein, 14 aa, Homo sapiens
TAHPSVSPRSAGQF

SEQ ID NO: 201 - protein, 14 aa, Homo sapiens
TAHPAPSPRSAGQF

SEQ ID NO: 202 - protein, 14 aa, Homo sapiens
TAHPRPSPRSAGQF

SEQ ID NO: 203 - protein, 14 aa, Homo sapiens
TAHPNPSPRSAGQF

SEQ ID NO: 204 - protein, 14 aa, Homo sapiens
TAHPDPSPRSAGQF

SEQ ID NO: 205 - protein, 14 aa, Homo sapiens
TAHPCPSPRSAGQF

SEQ ID NO: 206 - protein, 14 aa, Homo sapiens
TAHPQPSPRSAGQF

SEQ ID NO: 207 - protein, 14 aa, Homo sapiens
TAHPEPSPRSAGQF

SEQ ID NO: 208 - protein, 14 aa, Homo sapiens
TAHPGPSPRSAGQF

SEQ ID NO: 209 - protein, 14 aa, Homo sapiens
TAHPHPSPRSAGQF

SEQ ID NO: 210 - protein, 14 aa, Homo sapiens
TAHPIPSPRSAGQF

SEQ ID NO: 211 - protein, 14 aa, Homo sapiens
TAHPLPSPRSAGQF

SEQ ID NO: 212 - protein, 14 aa, Homo sapiens
TAHPKPSPRSAGQF

SEQ ID NO: 213 - protein, 14 aa, Homo sapiens
TAHPMPSPRSAGQF

SEQ ID NO: 214 - protein, 14 aa, Homo sapiens
TAHPFPSPRSAGQF

SEQ ID NO: 215 - protein, 14 aa, Homo sapiens
TAHPPPSPRSAGQF

SEQ ID NO: 216 - protein, 14 aa, Homo sapiens
TAHPSPSPRSAGQF

SEQ ID NO: 217 - protein, 14 aa, Homo sapiens
TAHPTPSPRSAGQF

SEQ ID NO: 218 - protein, 14 aa, Homo sapiens
TAHPWPSPRSAGQF

SEQ ID NO: 219 - protein, 14 aa, Homo sapiens
TAHPYPSPRSAGQF

SEQ ID NO: 220 - protein, 14 aa, Homo sapiens
TAHPVPSPRSAGQF

SEQ ID NO: 221 - protein, 14 aa, Homo sapiens
TAHASPSPRSAGQF

SEQ ID NO: 222 - protein, 14 aa, Homo sapiens
TAHRSPSPRSAGQF

SEQ ID NO: 223 - protein, 14 aa, Homo sapiens
TAHNSPSPRSAGQF

SEQ ID NO: 224 - protein, 14 aa, Homo sapiens
TAHDSPSPRSAGQF

SEQ ID NO: 225 - protein, 14 aa, Homo sapiens
TAHCSPSPRSAGQF

SEQ ID NO: 226 - protein, 14 aa, Homo sapiens
TAHQSPSPRSAGQF

SEQ ID NO: 227 - protein, 14 aa, Homo sapiens
TAHESPSPRSAGQF

SEQ ID NO: 228 - protein, 14 aa, Homo sapiens
TAHGSPSPRSAGQF

SEQ ID NO: 229 - protein, 14 aa, Homo sapiens
TAHHSPSPRSAGQF

SEQ ID NO: 230 - protein, 14 aa, Homo sapiens
TAHISPSPRSAGQF

SEQ ID NO: 231 - protein, 14 aa, Homo sapiens
TAHLSPSPRSAGQF

SEQ ID NO: 232 - protein, 14 aa, Homo sapiens
TAHKSPSPRSAGQF

SEQ ID NO: 233 - protein, 14 aa, Homo sapiens
TAHMSPSPRSAGQF

SEQ ID NO: 234 - protein, 14 aa, Homo sapiens
TAHFSPSPRSAGQF

SEQ ID NO: 235 - protein, 14 aa, Homo sapiens
TAHPSPSPRSAGQF

SEQ ID NO: 236 - protein, 14 aa, Homo sapiens
TAHSSPSPRSAGQF

SEQ ID NO: 237 - protein, 14 aa, Homo sapiens
TAHTSPSPRSAGQF

SEQ ID NO: 238 - protein, 14 aa, Homo sapiens
TAHWSPSPRSAGQF

SEQ ID NO: 239 - protein, 14 aa, Homo sapiens
TAHYSPSPRSAGQF

SEQ ID NO: 240 - protein, 14 aa, Homo sapiens
TAHVSPSPRSAGQF

SEQ ID NO: 241 - protein, 14 aa, Homo sapiens
TAAPSPSPRSAGQF

SEQ ID NO: 242 - protein, 14 aa, Homo sapiens
TARPSPSPRSAGQF

SEQ ID NO: 243 - protein, 14 aa, Homo sapiens
TANPSPSPRSAGQF

SEQ ID NO: 244 - protein, 14 aa, Homo sapiens
TADPSPSPRSAGQF

SEQ ID NO: 245 - protein, 14 aa, Homo sapiens
TACPSPSPRSAGQF

SEQ ID NO: 246 - protein, 14 aa, Homo sapiens
TAQPSPSPRSAGQF

SEQ ID NO: 247 - protein, 14 aa, Homo sapiens
TAEPSPSPRSAGQF

SEQ ID NO: 248 - protein, 14 aa, Homo sapiens
TAGPSPSPRSAGQF

SEQ ID NO: 249 - protein, 14 aa, Homo sapiens
TAHPSPSPRSAGQF

SEQ ID NO: 250 - protein, 14 aa, Homo sapiens
TAIPSPSPRSAGQF

SEQ ID NO: 251 - protein, 14 aa, Homo sapiens
TALPSPSPRSAGQF

SEQ ID NO: 252 - protein, 14 aa, Homo sapiens
TAKPSPSPRSAGQF

SEQ ID NO: 253 - protein, 14 aa, Homo sapiens
TAMPSPSPRSAGQF

SEQ ID NO: 254 - protein, 14 aa, Homo sapiens
TAFPSPSPRSAGQF

SEQ ID NO: 255 - protein, 14 aa, Homo sapiens
TAPPSPSPRSAGQF

SEQ ID NO: 256 - protein, 14 aa, Homo sapiens
TASPSPSPRSAGQF

SEQ ID NO: 257 - protein, 14 aa, Homo sapiens
TATPSPSPRSAGQF

SEQ ID NO: 258 - protein, 14 aa, Homo sapiens
TAWPSPSPRSAGQF

SEQ ID NO: 259 - protein, 14 aa, Homo sapiens
TAYPSPSPRSAGQF

SEQ ID NO: 260 - protein, 14 aa, Homo sapiens
TAVPSPSPRSAGQF

SEQ ID NO: 261 - protein, 14 aa, Homo sapiens
TAHPSPSPRSAGQF

SEQ ID NO: 262 - protein, 14 aa, Homo sapiens
TRHPSPSPRSAGQF

SEQ ID NO: 263 - protein, 14 aa, Homo sapiens
TNHPSPSPRSAGQF

SEQ ID NO: 264 - protein, 14 aa, Homo sapiens
TDHPSPSPRSAGQF

SEQ ID NO: 265 - protein, 14 aa, Homo sapiens
TCHPSPSPRSAGQF

SEQ ID NO: 266 - protein, 14 aa, Homo sapiens
TQHPSPSPRSAGQF

SEQ ID NO: 267 - protein, 14 aa, Homo sapiens
TEHPSPSPRSAGQF

SEQ ID NO: 268 - protein, 14 aa, Homo sapiens
TGHPSPSPRSAGQF

SEQ ID NO: 269 - protein, 14 aa, Homo sapiens
THHPSPSPRSAGQF

SEQ ID NO: 270 - protein, 14 aa, Homo sapiens
TIHPSPSPRSAGQF

SEQ ID NO: 271 - protein, 14 aa, Homo sapiens
TLHPSPSPRSAGQF

SEQ ID NO: 272 - protein, 14 aa, Homo sapiens
TKHPSPSPRSAGQF

SEQ ID NO: 273 - protein, 14 aa, Homo sapiens
TMHPSPSPRSAGQF

SEQ ID NO: 274 - protein, 14 aa, Homo sapiens
TFHPSPSPRSAGQF

SEQ ID NO: 275 - protein, 14 aa, Homo sapiens
TPHPSPSPRSAGQF

SEQ ID NO: 276 - protein, 14 aa, Homo sapiens
TSHPSPSPRSAGQF

SEQ ID NO: 277 - protein, 14 aa, Homo sapiens
TTHPSPSPRSAGQF

SEQ ID NO: 278 - protein, 14 aa, Homo sapiens
TWHPSPSPRSAGQF

SEQ ID NO: 279 - protein, 14 aa, Homo sapiens
TYHPSPSPRSAGQF

SEQ ID NO: 280 - protein, 14 aa, Homo sapiens
TVHPSPSPRSAGQF

SEQ ID NO: 281 - protein, 14 aa, Homo sapiens
AAHPSPSPRSAGQF

SEQ ID NO: 282 - protein, 14 aa, Homo sapiens
RAHPSPSPRSAGQF

SEQ ID NO: 283 - protein, 14 aa, Homo sapiens
NAHPSPSPRSAGQF

SEQ ID NO: 284 - protein, 14 aa, Homo sapiens
DAHPSPSPRSAGQF

SEQ ID NO: 285 - protein, 14 aa, Homo sapiens
CAHPSPSPRSAGQF

SEQ ID NO: 286 - protein, 14 aa, Homo sapiens
QAHPSPSPRSAGQF

SEQ ID NO: 287 - protein, 14 aa, Homo sapiens
EAHPSPSPRSAGQF

SEQ ID NO: 288 - protein, 14 aa, Homo sapiens
GAHPSPSPRSAGQF

SEQ ID NO: 289 - protein, 14 aa, Homo sapiens
HAHPSPSPRSAGQF

SEQ ID NO: 290 - protein, 14 aa, Homo sapiens
IAHPSPSPRSAGQF

SEQ ID NO: 291 - protein, 14 aa, Homo sapiens
LAHPSPSPRSAGQF

SEQ ID NO: 292 - protein, 14 aa, Homo sapiens
KAHPSPSPRSAGQF

SEQ ID NO: 293 - protein, 14 aa, Homo sapiens
MAHPSPSPRSAGQF

SEQ ID NO: 294 - protein, 14 aa, Homo sapiens
FAHPSPSPRSAGQF

SEQ ID NO: 295 - protein, 14 aa, Homo sapiens
PAHPSPSPRSAGQF

SEQ ID NO: 296 - protein, 14 aa, Homo sapiens
SAHPSPSPRSAGQF

SEQ ID NO: 297 - protein, 14 aa, Homo sapiens
TAHPSPSPRSAGQF

SEQ ID NO: 298 - protein, 14 aa, Homo sapiens
WAHPSPSPRSAGQF

SEQ ID NO: 299 - protein, 14 aa, Homo sapiens
YAHPSPSPRSAGQF

SEQ ID NO: 300 - protein, 14 aa, Homo sapiens
VAHPSPSPRSAGQF

SEQ ID NO: 301 - protein, 10 aa, Homo sapiens
WYRMSPSNQA

SEQ ID NO: 302 - protein, 10 aa, Homo sapiens
WYRMSPSNQR

SEQ ID NO: 303 - protein, 10 aa, Homo sapiens
WYRMSPSNQN

SEQ ID NO: 304 - protein, 10 aa, Homo sapiens
WYRMSPSNQD

SEQ ID NO: 305 - protein, 10 aa, Homo sapiens
WYRMSPSNQC

SEQ ID NO: 306 - protein, 10 aa, Homo sapiens
WYRMSPSNQQ

SEQ ID NO: 307 - protein, 10 aa, Homo sapiens
WYRMSPSNQE

SEQ ID NO: 308 - protein, 10 aa, Homo sapiens
WYRMSPSNQG

SEQ ID NO: 309 - protein, 10 aa, Homo sapiens
WYRMSPSNQH

SEQ ID NO: 310 - protein, 10 aa, Homo sapiens
WYRMSPSNQI

SEQ ID NO: 311 - protein, 10 aa, Homo sapiens
WYRMSPSNQL

SEQ ID NO: 312 - protein, 10 aa, Homo sapiens
WYRMSPSNQK

SEQ ID NO: 313 - protein, 10 aa, Homo sapiens
WYRMSPSNQM

SEQ ID NO: 314 - protein, 10 aa, Homo sapiens
WYRMSPSNQF

SEQ ID NO: 315 - protein, 10 aa, Homo sapiens
WYRMSPSNQP

SEQ ID NO: 316 - protein, 10 aa, Homo sapiens
WYRMSPSNQS

SEQ ID NO: 317 - protein, 10 aa, Homo sapiens
WYRMSPSNQT

SEQ ID NO: 318 - protein, 10 aa, Homo sapiens
WYRMSPSNQW

SEQ ID NO: 319 - protein, 10 aa, Homo sapiens
WYRMSPSNQY

SEQ ID NO: 320 - protein, 10 aa, Homo sapiens
WYRMSPSNQV

SEQ ID NO: 321 - protein, 10 aa, Homo sapiens
WYRMSPSNAT

SEQ ID NO: 322 - protein, 10 aa, Homo sapiens
WYRMSPSNRT

SEQ ID NO: 323 - protein, 10 aa, Homo sapiens
WYRMSPSNNT

SEQ ID NO: 324 - protein, 10 aa, Homo sapiens
WYRMSPSNDT

SEQ ID NO: 325 - protein, 10 aa, Homo sapiens
WYRMSPSNCT

SEQ ID NO: 326 - protein, 10 aa, Homo sapiens
WYRMSPSNQT

SEQ ID NO: 327 - protein, 10 aa, Homo sapiens
WYRMSPSNET

SEQ ID NO: 328 - protein, 10 aa, Homo sapiens
WYRMSPSNGT

SEQ ID NO: 329 - protein, 10 aa, Homo sapiens
WYRMSPSNHT

SEQ ID NO: 330 - protein, 10 aa, Homo sapiens
WYRMSPSNIT

SEQ ID NO: 331 - protein, 10 aa, Homo sapiens
WYRMSPSNLT

SEQ ID NO: 332 - protein, 10 aa, Homo sapiens
WYRMSPSNKT

SEQ ID NO: 333 - protein, 10 aa, Homo sapiens
WYRMSPSNMT

SEQ ID NO: 334 - protein, 10 aa, Homo sapiens
WYRMSPSNFT

SEQ ID NO: 335 - protein, 10 aa, Homo sapiens
WYRMSPSNPT

SEQ ID NO: 336 - protein, 10 aa, Homo sapiens
WYRMSPSNST

SEQ ID NO: 337 - protein, 10 aa, Homo sapiens
WYRMSPSNTT

SEQ ID NO: 338 - protein, 10 aa, Homo sapiens
WYRMSPSNWT

SEQ ID NO: 339 - protein, 10 aa, Homo sapiens
WYRMSPSNYT

SEQ ID NO: 340 - protein, 10 aa, Homo sapiens
WYRMSPSNVT

SEQ ID NO: 341 - protein, 10 aa, Homo sapiens
WYRMSPSAQT

SEQ ID NO: 342 - protein, 10 aa, Homo sapiens
WYRMSPSRQT

SEQ ID NO: 343 - protein, 10 aa, Homo sapiens
WYRMSPSNQT

SEQ ID NO: 344 - protein, 10 aa, Homo sapiens
WYRMSPSDQT

SEQ ID NO: 345 - protein, 10 aa, Homo sapiens
WYRMSPSCQT

SEQ ID NO: 346 - protein, 10 aa, Homo sapiens
WYRMSPSQQT

SEQ ID NO: 347 - protein, 10 aa, Homo sapiens
WYRMSPSEQT

SEQ ID NO: 348 - protein, 10 aa, Homo sapiens
WYRMSPSGQT

SEQ ID NO: 349 - protein, 10 aa, Homo sapiens
WYRMSPSHQT

SEQ ID NO: 350 - protein, 10 aa, Homo sapiens
WYRMSPSIQT

SEQ ID NO: 351 - protein, 10 aa, Homo sapiens
WYRMSPSLQT

SEQ ID NO: 352 - protein, 10 aa, Homo sapiens
WYRMSPSKQT

SEQ ID NO: 353 - protein, 10 aa, Homo sapiens
WYRMSPSMQT

SEQ ID NO: 354 - protein, 10 aa, Homo sapiens
WYRMSPSFQT

SEQ ID NO: 355 - protein, 10 aa, Homo sapiens
WYRMSPSPQT

SEQ ID NO: 356 - protein, 10 aa, Homo sapiens
WYRMSPSSQT

SEQ ID NO: 357 - protein, 10 aa, Homo sapiens
WYRMSPSTQT

SEQ ID NO: 358 - protein, 10 aa, Homo sapiens
WYRMSPSWQT

SEQ ID NO: 359 - protein, 10 aa, Homo sapiens
WYRMSPSYQT

SEQ ID NO: 360 - protein, 10 aa, Homo sapiens
WYRMSPSVQT

SEQ ID NO: 361 - protein, 10 aa, Homo sapiens
WYRMSPANQT

SEQ ID NO: 362 - protein, 10 aa, Homo sapiens
WYRMSPRNQT

SEQ ID NO: 363 - protein, 10 aa, Homo sapiens
WYRMSPNNQT

SEQ ID NO: 364 - protein, 10 aa, Homo sapiens
WYRMSPDNQT

SEQ ID NO: 365 - protein, 10 aa, Homo sapiens
WYRMSPCNQT

SEQ ID NO: 366 - protein, 10 aa, Homo sapiens
WYRMSPQNQT

SEQ ID NO: 367 - protein, 10 aa, Homo sapiens
WYRMSPENQT

SEQ ID NO: 368 - protein, 10 aa, Homo sapiens
WYRMSPGNQT

SEQ ID NO: 369 - protein, 10 aa, Homo sapiens
WYRMSPHNQT

SEQ ID NO: 370 - protein, 10 aa, Homo sapiens
WYRMSPINQT

SEQ ID NO: 371 - protein, 10 aa, Homo sapiens
WYRMSPLNQT

SEQ ID NO: 372 - protein, 10 aa, Homo sapiens
WYRMSPKNQT

SEQ ID NO: 373 - protein, 10 aa, Homo sapiens
WYRMSPMNQT

SEQ ID NO: 374 - protein, 10 aa, Homo sapiens
WYRMSPFNQT

SEQ ID NO: 375 - protein, 10 aa, Homo sapiens
WYRMSPPNQT

SEQ ID NO: 376 - protein, 10 aa, Homo sapiens
WYRMSPSNQT

SEQ ID NO: 377 - protein, 10 aa, Homo sapiens
WYRMSPTNQT

SEQ ID NO: 378 - protein, 10 aa, Homo sapiens
WYRMSPWNQT

SEQ ID NO: 379 - protein, 10 aa, Homo sapiens
WYRMSPYNQT

SEQ ID NO: 380 - protein, 10 aa, Homo sapiens
WYRMSPVNQT

SEQ ID NO: 381 - protein, 10 aa, Homo sapiens
WYRMSASNQT

SEQ ID NO: 382 - protein, 10 aa, Homo sapiens
WYRMSRSNQT

SEQ ID NO: 383 - protein, 10 aa, Homo sapiens
WYRMSNSNQT

SEQ ID NO: 384 - protein, 10 aa, Homo sapiens
WYRMSDSNQT

SEQ ID NO: 385 - protein, 10 aa, Homo sapiens
WYRMSCSNQT

SEQ ID NO: 386 - protein, 10 aa, Homo sapiens
WYRMSQSNQT

SEQ ID NO: 387 - protein, 10 aa, Homo sapiens
WYRMSESNQT

SEQ ID NO: 388 - protein, 10 aa, Homo sapiens
WYRMSGSNQT

SEQ ID NO: 389 - protein, 10 aa, Homo sapiens
WYRMSHSNQT

SEQ ID NO: 390 - protein, 10 aa, Homo sapiens
WYRMSISNQT

SEQ ID NO: 391 - protein, 10 aa, Homo sapiens
WYRMSLSNQT

SEQ ID NO: 392 - protein, 10 aa, Homo sapiens
WYRMSKSNQT

SEQ ID NO: 393 - protein, 10 aa, Homo sapiens
WYRMSMSNQT

SEQ ID NO: 394 - protein, 10 aa, Homo sapiens
WYRMSFSNQT

SEQ ID NO: 395 - protein, 10 aa, Homo sapiens
WYRMSPSNQT

SEQ ID NO: 396 - protein, 10 aa, Homo sapiens
WYRMSSSNQT

SEQ ID NO: 397 - protein, 10 aa, Homo sapiens
WYRMSTSNQT

SEQ ID NO: 398 - protein, 10 aa, Homo sapiens
WYRMSWSNQT

SEQ ID NO: 399 - protein, 10 aa, Homo sapiens
WYRMSYSNQT

SEQ ID NO: 400 - protein, 10 aa, Homo sapiens
WYRMSVSNQT

SEQ ID NO: 401 - protein, 10 aa, Homo sapiens
WYRMAPSNQT

SEQ ID NO: 402 - protein, 10 aa, Homo sapiens
WYRMRPSNQT

SEQ ID NO: 403 - protein, 10 aa, Homo sapiens
WYRMNPSNQT

SEQ ID NO: 404 - protein, 10 aa, Homo sapiens
WYRMDPSNQT

SEQ ID NO: 405 - protein, 10 aa, Homo sapiens
WYRMCPSNQT

SEQ ID NO: 406 - protein, 10 aa, Homo sapiens
WYRMQPSNQT

SEQ ID NO: 407 - protein, 10 aa, Homo sapiens
WYRMEPSNQT

SEQ ID NO: 408 - protein, 10 aa, Homo sapiens
WYRMGPSNQT

SEQ ID NO: 409 - protein, 10 aa, Homo sapiens
WYRMHPSNQT

SEQ ID NO: 410 - protein, 10 aa, Homo sapiens
WYRMIPSNQT

SEQ ID NO: 411 - protein, 10 aa, Homo sapiens
WYRMLPSNQT

SEQ ID NO: 412 - protein, 10 aa, Homo sapiens
WYRMKPSNQT

SEQ ID NO: 413 - protein, 10 aa, Homo sapiens
WYRMMPSNQT

SEQ ID NO: 414 - protein, 10 aa, Homo sapiens
WYRMFPSNQT

SEQ ID NO: 415 - protein, 10 aa, Homo sapiens
WYRMPPSNQT

SEQ ID NO: 416 - protein, 10 aa, Homo sapiens
WYRMSPSNQT

SEQ ID NO: 417 - protein, 10 aa, Homo sapiens
WYRMTPSNQT

SEQ ID NO: 418 - protein, 10 aa, Homo sapiens
WYRMWPSNQT

SEQ ID NO: 419 - protein, 10 aa, Homo sapiens
WYRMYPSNQT

SEQ ID NO: 420 - protein, 10 aa, Homo sapiens
WYRMVPSNQT

SEQ ID NO: 421 - protein, 10 aa, Homo sapiens
WYRASPSNQT

SEQ ID NO: 422 - protein, 10 aa, Homo sapiens
WYRRSPSNQT

SEQ ID NO: 423 - protein, 10 aa, Homo sapiens
WYRNSPSNQT

SEQ ID NO: 424 - protein, 10 aa, Homo sapiens
WYRDSPSNQT

SEQ ID NO: 425 - protein, 10 aa, Homo sapiens
WYRCSPSNQT

SEQ ID NO: 426 - protein, 10 aa, Homo sapiens
WYRQSPSNQT

SEQ ID NO: 427 - protein, 10 aa, Homo sapiens
WYRESPSNQT

SEQ ID NO: 428 - protein, 10 aa, Homo sapiens
WYRGSPSNQT

SEQ ID NO: 429 - protein, 10 aa, Homo sapiens
WYRHSPSNQT

SEQ ID NO: 430 - protein, 10 aa, Homo sapiens
WYRISPSNQT

SEQ ID NO: 431 - protein, 10 aa, Homo sapiens
WYRLSPSNQT

SEQ ID NO: 432 - protein, 10 aa, Homo sapiens
WYRKSPSNQT

SEQ ID NO: 433 - protein, 10 aa, Homo sapiens
WYRMSPSNQT

SEQ ID NO: 434 - protein, 10 aa, Homo sapiens
WYRFSPSNQT

SEQ ID NO: 435 - protein, 10 aa, Homo sapiens
WYRPSPSNQT

SEQ ID NO: 436 - protein, 10 aa, Homo sapiens
WYRSSPSNQT

SEQ ID NO: 437 - protein, 10 aa, Homo sapiens
WYRTSPSNQT

SEQ ID NO: 438 - protein, 10 aa, Homo sapiens
WYRWSPSNQT

SEQ ID NO: 439 - protein, 10 aa, Homo sapiens
WYRYSPSNQT

SEQ ID NO: 440 - protein, 10 aa, Homo sapiens
WYRVSPSNQT

SEQ ID NO: 441 - protein, 10 aa, Homo sapiens
WYAMSPSNQT

SEQ ID NO: 442 - protein, 10 aa, Homo sapiens
WYRMSPSNQT

SEQ ID NO: 443 - protein, 10 aa, Homo sapiens
WYNMSPSNQT

SEQ ID NO: 444 - protein, 10 aa, Homo sapiens
WYDMSPSNQT

SEQ ID NO: 445 - protein, 10 aa, Homo sapiens
WYCMSPSNQT

SEQ ID NO: 446 - protein, 10 aa, Homo sapiens
WYQMSPSNQT

SEQ ID NO: 447 - protein, 10 aa, Homo sapiens
WYEMSPSNQT

SEQ ID NO: 448 - protein, 10 aa, Homo sapiens
WYGMSPSNQT

SEQ ID NO: 449 - protein, 10 aa, Homo sapiens
WYHMSPSNQT

SEQ ID NO: 450 - protein, 10 aa, Homo sapiens
WYIMSPSNQT

SEQ ID NO: 451 - protein, 10 aa, Homo sapiens
WYLMSPSNQT

SEQ ID NO: 452 - protein, 10 aa, Homo sapiens
WYKMSPSNQT

SEQ ID NO: 453 - protein, 10 aa, Homo sapiens
WYMMSPSNQT

SEQ ID NO: 454 - protein, 10 aa, Homo sapiens
WYFMSPSNQT

SEQ ID NO: 455 - protein, 10 aa, Homo sapiens
WYPMSPSNQT

SEQ ID NO: 456 - protein, 10 aa, Homo sapiens
WYSMSPSNQT

SEQ ID NO: 457 - protein, 10 aa, Homo sapiens
WYTMSPSNQT

SEQ ID NO: 458 - protein, 10 aa, Homo sapiens
WYWMSPSNQT

SEQ ID NO: 459 - protein, 10 aa, Homo sapiens
WYYMSPSNQT

SEQ ID NO: 460 - protein, 10 aa, Homo sapiens
WYVMSPSNQT

SEQ ID NO: 461 - protein, 10 aa, Homo sapiens
WARMSPSNQT

SEQ ID NO: 462 - protein, 10 aa, Homo sapiens
WRRMSPSNQT

SEQ ID NO: 463 - protein, 10 aa, Homo sapiens
WNRMSPSNQT

SEQ ID NO: 464 - protein, 10 aa, Homo sapiens
WDRMSPSNQT

SEQ ID NO: 465 - protein, 10 aa, Homo sapiens
WCRMSPSNQT

SEQ ID NO: 466 - protein, 10 aa, Homo sapiens
WQRMSPSNQT

SEQ ID NO: 467 - protein, 10 aa, Homo sapiens
WERMSPSNQT

SEQ ID NO: 468 - protein, 10 aa, Homo sapiens
WGRMSPSNQT

SEQ ID NO: 469 - protein, 10 aa, Homo sapiens
WHRMSPSNQT

SEQ ID NO: 470 - protein, 10 aa, Homo sapiens
WIRMSPSNQT

SEQ ID NO: 471 - protein, 10 aa, Homo sapiens
WLRMSPSNQT

SEQ ID NO: 472 - protein, 10 aa, Homo sapiens
WKRMSPSNQT

SEQ ID NO: 473 - protein, 10 aa, Homo sapiens
WMRMSPSNQT

SEQ ID NO: 474 - protein, 10 aa, Homo sapiens
WFRMSPSNQT

SEQ ID NO: 475 - protein, 10 aa, Homo sapiens
WPRMSPSNQT

SEQ ID NO: 476 - protein, 10 aa, Homo sapiens
WSRMSPSNQT

SEQ ID NO: 477 - protein, 10 aa, Homo sapiens
WTRMSPSNQT

SEQ ID NO: 478 - protein, 10 aa, Homo sapiens
WWRMSPSNQT

SEQ ID NO: 479 - protein, 10 aa, Homo sapiens
WYRMSPSNQT

SEQ ID NO: 480 - protein, 10 aa, Homo sapiens
WVRMSPSNQT

SEQ ID NO: 481 - protein, 10 aa, Homo sapiens
AYRMSPSNQT

SEQ ID NO: 482 - protein, 10 aa, Homo sapiens
RYRMSPSNQT

SEQ ID NO: 483 - protein, 10 aa, Homo sapiens
NYRMSPSNQT

SEQ ID NO: 484 - protein, 10 aa, Homo sapiens
DYRMSPSNQT

SEQ ID NO: 485 - protein, 10 aa, Homo sapiens
CYRMSPSNQT

SEQ ID NO: 486 - protein, 10 aa, Homo sapiens
QYRMSPSNQT

SEQ ID NO: 487 - protein, 10 aa, Homo sapiens
EYRMSPSNQT

SEQ ID NO: 488 - protein, 10 aa, Homo sapiens
GYRMSPSNQT

SEQ ID NO: 489 - protein, 10 aa, Homo sapiens
HYRMSPSNQT

SEQ ID NO: 490 - protein, 10 aa, Homo sapiens
IYRMSPSNQT

SEQ ID NO: 491 - protein, 10 aa, Homo sapiens
LYRMSPSNQT

SEQ ID NO: 492 - protein, 10 aa, Homo sapiens
KYRMSPSNQT

SEQ ID NO: 493 - protein, 10 aa, Homo sapiens
MYRMSPSNQT

SEQ ID NO: 494 - protein, 10 aa, Homo sapiens
FYRMSPSNQT

SEQ ID NO: 495 - protein, 10 aa, Homo sapiens
PYRMSPSNQT

SEQ ID NO: 496 - protein, 10 aa, Homo sapiens
SYRMSPSNQT

SEQ ID NO: 497 - protein, 10 aa, Homo sapiens
TYRMSPSNQT

SEQ ID NO: 498 - protein, 10 aa, Homo sapiens
WYRMSPSNQT

SEQ ID NO: 499 - protein, 10 aa, Homo sapiens
YYRMSPSNQT

SEQ ID NO: 500 - protein, 10 aa, Homo sapiens
VYRMSPSNQT

SEQ ID NO: 501 - protein, 20 aa, Homo sapiens
METDTLLLWVLLLWVPGSTG

SEQ ID NO: 502 - protein, 20 aa, Homo sapiens
MYRMQLLSCIALSLALVTNS

SEQ ID NO: 503 - protein, 15 aa, synthetic construct
  REGION 1..15: note = Synthetic sequence
GGGGSGGGGSGGGGS

SEQ ID NO: 504 - protein, 16 aa, synthetic construct
  REGION 1..16: note = Synthetic sequence
GGGGSLVPRGSGGGGS

SEQ ID NO: 505 - protein, 6 aa, synthetic construct
  REGION 1..6: note = Synthetic sequence
GSGSGS

SEQ ID NO: 506 - protein, 8 aa, synthetic construct
  REGION 1..8: note = Synthetic sequence
GSGSGSGS

SEQ ID NO: 507 - protein, 14 aa, synthetic construct
  REGION 1..14: note = Synthetic sequence
GGGGSLVPRGSGGG

SEQ ID NO: 508 - protein, 10 aa, synthetic construct
  REGION 1..10: note = Synthetic sequence
GSGGHMGSGG

SEQ ID NO: 509 - protein, 11 aa, synthetic construct
  REGION 1..11: note = Synthetic sequence
GGSGGSGGSGG

SEQ ID NO: 510 - protein, 5 aa, synthetic construct
  REGION 1..5: note = Synthetic sequence
GGSGG

SEQ ID NO: 511 - protein, 8 aa, synthetic construct
  REGION 1..8: note = Synthetic sequence
GGSGGGGG

SEQ ID NO: 512 - protein, 18 aa, synthetic construct
  REGION 1..18: note = Synthetic sequence
GGGSEGGGSEGGGSEGGG

SEQ ID NO: 513 - protein, 8 aa, synthetic construct
  REGION 1..8: note = Synthetic sequence
AAGAATAA

SEQ ID NO: 514 - protein, 5 aa, synthetic construct
  REGION 1..5: note = Synthetic sequence
GGGGG

SEQ ID NO: 515 - protein, 5 aa, synthetic construct
  REGION 1..5: note = Synthetic sequence
GGSSG

SEQ ID NO: 516 - protein, 11 aa, synthetic construct
  REGION 1..11: note = Synthetic sequence
GSGGGTGGGSG

SEQ ID NO: 517 - skipped

SEQ ID NO: 518 - protein, 26 aa, synthetic construct
  REGION 1..26: note = Synthetic sequence
GSGSGSGSGGSGGSGGSGGSGGSGGS

SEQ ID NO: 519 - protein, 14 aa, synthetic construct
  REGION 1..14: note = Synthetic sequence
GSGGSGSGGSGGSG

SEQ ID NO: 520 - protein, 22 aa, synthetic construct
  REGION 1..22: note = Synthetic sequence
VKQTLNFDLLKLAGDVESNPGP

SEQ ID NO: 521 - protein, 20 aa, synthetic construct
  REGION 1..20: note = Synthetic sequence
QCTNYALLKLAGDVESNPGP

SEQ ID NO: 522 - protein, 19 aa, synthetic construct
  REGION 1..19: note = Synthetic sequence
ATNFSLLKQAGDVEENPGP

SEQ ID NO: 523 - protein, 20 aa, synthetic construct
  REGION 1..20: note = Synthetic sequence
HYAGYFADLLIHDIETNPGP

SEQ ID NO: 524 - protein, 25 aa, synthetic construct
  REGION 1..25: note = Synthetic sequence
GIFNAHYAGYFADLLIHDIETNPGP

SEQ ID NO: 525 - protein, 25 aa, synthetic construct
  REGION 1..25: note = Synthetic sequence
KAVRGYHADYYKQRLIHDVEMNPGP

SEQ ID NO: 526 - protein, 20 aa, synthetic construct
  REGION 1..20: note = Synthetic sequence
GATNFSLLKLAGDVELNPGP

SEQ ID NO: 527 - protein, 18 aa, synthetic construct
  REGION 1..18: note = Synthetic sequence
EGRGSLLTCGDVEENPGP

SEQ ID NO: 528 - protein, 19 aa, synthetic construct
  REGION 1..19: note = Synthetic sequence
AARQMLLLLSGDVETNPGP

SEQ ID NO: 529 - protein, 20 aa, synthetic construct
  REGION 1..20: note = Synthetic sequence
FLRKRTQLLMSGDVESNPGP

SEQ ID NO: 530 - protein, 20 aa, synthetic construct
  REGION 1..20: note = Synthetic sequence
GSWTDILLLLSGDVETNPGP

SEQ ID NO: 531 - protein, 19 aa, synthetic construct
  REGION 1..19: note = Synthetic sequence
TRAEEDELIRAGIESNPGP

SEQ ID NO: 532 - protein, 20 aa, synthetic construct
  REGION 1..20: note = Synthetic sequence
AKFQIDKILISGDVELNPGP

SEQ ID NO: 533 - protein, 20 aa, synthetic construct
  REGION 1..20: note = Synthetic sequence
SKFQIDKILISGDIELNPGP

SEQ ID NO: 534 - protein, 20 aa, synthetic construct
  REGION 1..20: note = Synthetic sequence
SSIIRTKMLVSGDVEENPGP

SEQ ID NO: 535 - protein, 20 aa, synthetic construct
  REGION 1..20: note = Synthetic sequence
CDAQRQKLLLSGDIEQNPGP

SEQ ID NO: 536 - DNA, 668 bp, synthetic construct
  misc_feature 1..668: note = Synthetic sequence
  source 1..668: mol_type = other DNA
gcgatctgacggttcactaaacgagctctgcttatataggcctcccaccgtacacgccacctcgacatactcgagtagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgct

SEQ ID NO: 537 - protein, 34 aa, synthetic construct
  REGION 1..34: note = Synthetic sequence
METDTLLLWVLLLWVPGSTGTAHPSPSPRSAGQF

SEQ ID NO: 538 - protein, 59 aa, synthetic construct
  REGION 1..59: note = Synthetic sequence
METDTLLLWVLLLWVPGSTGEYRMSPSNQTGGGGSGGGGSGGGGSTAHPSPSPRSAGQF

SEQ ID NO: 539 - protein, 59 aa, synthetic construct
  REGION 1..59: note = Synthetic sequence
METDTLLLWVLLLWVPGSTGYYRMSPSNQTGGGGSGGGGSGGGGSTAHPSPSPRSAGQF

SEQ ID NO: 540 - protein, 34 aa, synthetic construct
  REGION 1..34: note = Synthetic sequence
METDTLLLWVLLLWVPGSTGTRYPSPSPKPEGRF

SEQ ID NO: 541 - protein, 59 aa, synthetic construct
  REGION 1..59: note = Synthetic sequence
METDTLLLWVLLLWVPGSTGWNRLSPSNQTGGGGSGGGGSGGGGSTRYPSPSPKPEGRF

SEQ ID NO: 542 - protein, 107 aa, synthetic construct
  REGION 1..107: note = Synthetic sequence
METDTLLLWVLLLWVPGSTGTAHPSPSPRSAGQFTAHPSPSPRSAGQFTAHPSPSPRSAGQFGGGGSGGGGSGGGGSEYRMSPSNQTEYRMSPSNQTEYRMSPSNQT

SEQ ID NO: 543 - protein, 107 aa, synthetic construct
  REGION 1..107: note = Synthetic sequence
METDTLLLWVLLLWVPGSTGEYRMSPSNQTEYRMSPSNQTEYRMSPSNQTGGGGSGGGGSGGGGSTAHPSPSPRSAGQFTAHPSPSPRSAGQFTAHPSPSPRSAGQF

SEQ ID NO: 544 - protein, 231 aa, Homo sapiens
ISAMVRSPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 545 - protein, 229 aa, Mus musculus
ISAMVRSGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 546 - protein, 265 aa, synthetic construct
  REGION 1..265: note = Synthetic sequence
MYRMQLLSCIALSLALVTNSTAHPSPSPRSAGQFISAMVRSPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 547 - protein, 290 aa, synthetic construct
  REGION 1..290: note = Synthetic sequence
MYRMQLLSCIALSLALVTNSEYRMSPSNQTGGGGSGGGGSGGGGSTAHPSPSPRSAGQFISAMVRSPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 548 - protein, 290 aa, synthetic construct
  REGION 1..290: note = Synthetic sequence
MYRMQLLSCIALSLALVTNSYYRMSPSNQTGGGGSGGGGSGGGGSTAHPSPSPRSAGQFISAMVRSPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 549 - protein, 263 aa, synthetic construct
  REGION 1..263: note = Synthetic sequence
MYRMQLLSCIALSLALVTNSTRYPSPSPKPEGRFISAMVRSGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 550 - protein, 288 aa, synthetic construct
  REGION 1..288: note = Synthetic sequence
MYRMQLLSCIALSLALVTNSWNRLSPSNQTGGGGSGGGGSGGGGSTRYPSPSPKPEGRFISAMVRSGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 551 - DNA, 1335 bp, Homo sapiens
  source 1..1335: mol_type = genomic DNA
gagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 552 - DNA, 601 bp, synthetic construct
  misc_feature 1..601: note = Synthetic sequence
  source 1..601: mol_type = other DNA
cctctcccccccccccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgccacaaccc

SEQ ID NO: 553 - DNA, 536 bp, Human cytomegalovirus
  source 1..536: mol_type = genomic DNA
ccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgct

SEQ ID NO: 554 - protein, 22 aa, synthetic construct
  REGION 1..22: note = Synthetic sequence
GSGATNFSLLKQAGDVEENPGP

SEQ ID NO: 555 - DNA, 1433 bp, synthetic construct
  misc_feature 1..1433: note = Synthetic sequence
  source 1..1433: mol_type = other DNA
atgtcgggcgtcgggggagagggagttccctctgcgcttgcgattctagcctcgtggggctggacgttcgacacgccaaaccacgagtcggggatatcgccagatacgactcccgcagattccattcggggtgccgctgtggcctcacctgaccaacctttacacgggggcccggaacgggaggccacagcgccgtctttctccccaacgcgcgcggatgacggcccgccctgtaccgacgggccctacgtgacgtttgataccctgtttatggtgtcgtcgatcgacgaattagggcgtcgccagctcacggacaccatccgcaaggacctgcggttgtcgctggccaagtttagcattgcgtgcaccaagacctcctcgttttcgggaaacgccccgcgccaccacagacgcggggcgttccagcgcggcacgcgggcgccgcgcagcaacaaaagcctccagatgtttgtgttgtgcaaacgcgcccacgccgctcgagtgcgagagcagcttcgggtcgttattcagtcccgcaagccgcgcaagtattacacgcgatcttcggacgggcggctctgccccgccgtccccgtgttcgtccacgagttcgtctcgtccgagccaatgcgcctccaccgagataacgtcatgctggcctcgggggccgagtaaccgccccccccccatgccaccctcactgcccgtcgcgcgtgtttgatgttaataaataacacataaatttggctggttgtttgttgtctttaatggaccgcccgcaagggggggggggcatttcagtgtcgggtgacgagcgcgatccggccgggatcctaggaccccaaaagtttgtctgcgtattccagggcggggctcagttgaatctcccgcagcacctctaccagcaggtccgcggtgggctggagaaactcggccgtcccggggcaggcggttgtcgggggtggaggcgcggcgcccaccccgtgtgccgcgcctggcgtctcctctgggggcgacccgtaaatggttgcagtgatgtaaatggtgtccgcggtccagaccacggtcaaaatgccggccgtggcgctccgggcgctttcgccgcgcgaggagctgacccaggagtcgaacggatacgcgtacatatgggcgtcccacccgcgttcgagcttctggttgctgtcccggcctataaagcggtaggcacaaaattcggcgcgacagtcgataatcaccaacagcccaatgggggtgtgctggataacaacgcctccgcgcggcaggcggtcctggcgctcccggccccgtaccatgatcgcgcgggtgccgtactcaaaaacatgcaccacctgcgcggcgtcgggcagtgcgctggtcagcgaggccctggcgtggcataggctatacgcgatggtcgtctgtggattggacat

SEQ ID NO: 556 - DNA, 1533 bp, synthetic construct
  misc_feature 1..1533: note = Synthetic sequence
  source 1..1533: mol_type = other DNA
ggccctcaatccctaatacatttcagtgatggggtagtccagtgagccacatttgttgccttgttcagcacgcactctgtcagggagctggtgccggccttccgcttgaagccagagttgcagatgtatctctcccgggaatacaggctgtaagacttcacccagatgtctgcgtgctccacgctcattggaggtgggcaggtgatgccccttgtagcggggggtctcagcagcagcagcagcagcagggcaggcagtcccagggtccgacatcctctagcgcggcgtggagccatggtggcgaattctccaggcgatctgacggttcactaaacgagctctgcttatataggcctcccaccgtacacgccacctcgacatactcgagtagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagggatcctctagtcagctgacgcgtgctagcgcggccgcatcgataagcttgccaccatgcggatttccaagccccatctgcgctccatttccatccagtgttacctgtgcctgctgctgaatagccattttctgaccgaagccggcatccacgtgttcatcctgggctgcttttccgccggcctgccaaagaccgaggcaaactgggtgaatgtgatctctgacctgaagaagatcgaggatctgatccagagcatgcacatcgacgccaccctgtacacagagtccgatgtgcacccttcttgcaaggtgacagccatgaagtgtttcctgctggagctgcaggtcatctctctggagagcggcgacgcctctatccacgataccgtggagaacctgatcatcctggccaacaatagcctgagctccaacggcaatgtgacagagtccggctgcaaggagtgtgaggagctggaggagaagaatatcaaagagttcctgcagagtttcgtccatatcgtccagatgtttatcaatacttcctaagtcga

SEQ ID NO: 557 - DNA, 2304 bp, synthetic construct
  misc_feature 1..2304: note = Synthetic sequence
  source 1..2304: mol_type = other DNA
ggcccttacagatggtgactacagttttccaggtcctcgtctcttgatgaagttccccaggtgacgggcagggcctccatggcctccatctccacagaggccagagggggggtctgtctggacttcagataacaggccagcagggacacggcgctcaggccgcacagcagcactgtagaggtggagatggccactgtggtgtcggagtggccctgtgggtacacgcctggaggctggtggctggcagatgctgtcagctcccaattcttggctgtggtctgagaaggggtgccgtggctagactcgtgggagctgatctctgtggtgcctgtggatgggctcttagaaggcatcagctgggagccaggcacgattgctgctgtggttgctgcggtattgttagaggatgggctagatgctgcaggctccttgccggatgggctcagagactctggctgtggtgtcacgccggctgtggtcacggtgctgggtggtgcgggccgctggtgcaccagggcgggatcccggatgcacttcagagagggtgtggtccagtgggccacattggtggccttgttcagcacgcactctgtcagggagctggtgccggccttcctcttaaagccgctgttgcagatgtatctctcccgagaatacagggagtagctcttcacccagatgtcggcgtgctccacggacatagggggtggacatgtgatgcctgcctgctgtctgctggtccggtggtggtgacactgggggcaattctccattgtcctcagcctcacgcatgcctcgtgggtctgcacctcgtggttgaaagactcggagattctgctccggcctgccagcacggccaggcgatcccttgcgtcgcgtgttgcagggggcctcagcagcagcagtaataacagggcgggcaggcccagggtgcgacagcccctggctctccttggtgccattgctgctgctccgcaggacacggcgggtgagccgggtcttgcgctgcccagtccgggtggtggaggggatctctgttcggggacctgtcttcctgccagtctcatggtggcgaattctccaggcgatctgacggttcactaaacgagctctgcttatataggcctcccaccgtacacgccacctcgacatactcgagtagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagggatcctctagtcagctgacgcgtgctagcgcggccgcatcgataagcttgccaccatgcggatttccaagccccatctgcgctccatttccatccagtgttacctgtgcctgctgctgaatagccattttctgaccgaagccggcatccacgtgttcatcctgggctgcttttccgccggcctgccaaagaccgaggcaaactgggtgaatgtgatctctgacctgaagaagatcgaggatctgatccagagcatgcacatcgacgccaccctgtacacagagtccgatgtgcacccttcttgcaaggtgacagccatgaagtgtttcctgctggagctgcaggtcatctctctggagagcggcgacgcctctatccacgataccgtggagaacctgatcatcctggccaacaatagcctgagctccaacggcaatgtgacagagtccggctgcaaggagtgtgaggagctggaggagaagaatatcaaagagttcctgcagagtttcgtccatatcgtccagatgtttatcaatacttcctaagtcga

SEQ ID NO: 558 - DNA, 1842 bp, synthetic construct
  misc_feature 1..1842: note = Synthetic sequence
  source 1..1842: mol_type = other DNA
ggcccttatttttccagtgcggacacctctttttccagggctgacacctccttttccagggctgagacttctttttccagggcagacacctccttctccagggcagacacctcggagccgccgccgccggatcctcctcctccgcttcctcctcctccatcccggatacacttcagggagggtgtggtccagtgggccacatttgtggccttgttcagcacgcactctgtcagggagctggtgccggccttcctcttgaagccgctgttgcagatgtatctctcccgagaatacaggctgtaagacttcacccagatgtctgcgtgctccacgctcattggaggtgggcaggtgatgccccttgtagcaggtggtctcagcagcagcagcagcagcagggcaggcagtcccagtgtgcgacatcctcgggctcttctaggggccatggtggcgaattctccaggcgatctgacggttcactaaacgagctctgcttatataggcctcccaccgtacacgccacctcgacatactcgagtagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagggatcctctagtcagctgacgcgtgctagcgcggccgcatcgataagcttgccaccatgcggatttccaaacctcacctgcgatctatctccatccagtgctatctgtgcctgctgctgaactctcatttcctgaccgaagccggcatccacgtgttcatcctgggctgctttagcgccggcctgccaaagaccgaggcaaactgggtgaatgtgatctctgacctgaagaagatcgaggatctgatccagagcatgcacatcgacgccaccctgtacacagagtccgatgtgcacccttcttgcaaggtgacagccatgaagtgtttcctgctggagctgcaggtcatctctctggagagcggcgacgcctccatccacgataccgtggagaacctgatcatcctggccaacaatagcctgagctccaacggcaatgtgacagagtccggctgcaaggagtgtgaggagctggaggagaagaacatcaaggagttcctgcagtcttttgtgcacatcgtgcagatgtttatcaataccagcggaggaggaggatccggcggaggaggctctggcggcggcggcagctgtggcggcaaggtgtccgccctgaaggagaaggtgtctgccctgaaggaaaaagtgtccgctctgaaggaaaaggtgtccgcactgaaagaaaaggtctccgccctgaaggaataagtcga

SEQ ID NO: 559 - DNA, 2613 bp, synthetic construct
  misc_feature 1..2613: note = Synthetic sequence
  source 1..2613: mol_type = other DNA
ggcccttatttttccagtgcggacacctctttttccagtgctgacacctccttctccagggctgacacttctttttccagggcgctcacctctttttccagggcagacacctcggagccgccgccgccggagcctcctcctccgcttcctcctcctcccaggtggtgggaacaattctccagatcctcgtcgcgagaggatgtgccccaggtcactggcagggcctccattgcctccatctccacagatgccagaggtggggtctgcctggacttcagataacaggccagcaggctcacggcagacaggccgcacagcagcactgtggaggtgctgatggccactgtggtgtcggagtggccctgtgggtacacgcctggaggctggtgagaggcggatgctgtcagctcccagttcttggctgtggtctgagaaggggtgccgtggctagactcgtgggagctgatctctgtggtgccggtgcttggagacttggaaggcatcagctgggagccaggcacgattgctgctgtggttgctgcggtattgttagaggatgggctagatgctgcaggctccttgccgcttggagacagggactctggctgtggtgtcactcctgctgtggtcacggtgctgggtggtgcgggccgctggtgcaccagggcgggatcccggatgcacttcagagagggtgtggtccagtgggccacattggtggccttgttcagcacgcactctgtcagggagctggtgccggccttccgcttaaagccgctgttgcagatgtatctctcccgagaatacaggctgtaagacttcacccagatgtcggcgtgctccacggacatagggggtggacatgtgatgcctgcctgctgtctgctggtccggtggtggtgacactgggggcaattctccattgtcctcagcctcacgcatgcctcgtgggtctgcacctcgtggttgaaggactcgctgattctgctccggcctgccagcacggccaggcgatcccttgcgtcgcgtgttgcagggggcctcagcagcagcagtaataacagggcgggcaggcccagggtgcgacagcccctggctctccttggtgccattgctgctgctccgcaggacacagcgggactgccgggtcttgcactgcccagcccaggaggaggaggggatctctgctcggggacctgccttccagccagccgcatggtggcgaattctccaggcgatctgacggttcactaaacgagctctgcttatataggcctcccaccgtacacgccacctcgacatactcgagtagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagggatcctctagtcagctgacgcgtgctagcgcggccgcatcgataagcttgccaccatgcggatttccaaacctcacctgcgatctatctccatccagtgctatctgtgcctgctgctgaactctcatttcctgaccgaagccggcatccacgtgttcatcctgggctgctttagcgccggcctgccaaagaccgaggcaaactgggtgaatgtgatctctgacctgaagaagatcgaggatctgatccagagcatgcacatcgacgccaccctgtacacagagtccgatgtgcacccttcttgcaaggtgacagccatgaagtgtttcctgctggagctgcaggtcatctctctggagagcggcgacgcctccatccacgataccgtggagaacctgatcatcctggccaacaatagcctgagctccaacggcaatgtgacagagtccggctgcaaggagtgtgaggagctggaggagaagaacatcaaggagttcctgcagtcttttgtgcacatcgtgcagatgtttatcaataccagcggaggaggaggatccggcggaggaggctctggcggcggcggcagctgtggcggcaaggtgtccgccctgaaggagaaggtgtctgccctgaaggaaaaagtgtccgctctgaaggaaaaggtgtccgcactgaaagaaaaggtctccgccctgaaggaataagtcga

SEQ ID NO: 560 - DNA, 3135 bp, synthetic construct
  misc_feature 1..3135: note = Synthetic sequence
  source 1..3135: mol_type = other DNA
gagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaactagaagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcgccaccatgcggatttccaagccccatctgcgctccatttccatccagtgttacctgtgcctgctgctgaatagccattttctgaccgaagccggcatccacgtgttcatcctgggctgcttttccgccggcctgccaaagaccgaggcaaactgggtgaatgtgatctctgacctgaagaagatcgaggatctgatccagagcatgcacatcgacgccaccctgtacacagagtccgatgtgcacccttcttgcaaggtgacagccatgaagtgtttcctgctggagctgcaggtcatctctctggagagcggcgacgcctctatccacgataccgtggagaacctgatcatcctggccaacaatagcctgagctccaacggcaatgtgacagagtccggctgcaaggagtgtgaggagctggaggagaagaatatcaaagagttcctgcagagtttcgtccatatcgtccagatgtttatcaatacttcctaagaattcacgcgtcgagcatgcatctagggcggccaattccgcccctctcccccccccccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgccacaacccgggatcctctagagtcgacgccaccatggctccacgccgcgctagaggatgtcggaccctgggactgcctgccctgctgctgctgctgctgctgagaccccccgctacaaggggcatcacctgcccacctccaatgagcgtggagcacgcagacatctgggtgaagtcttacagcctgtattcccgggagagatacatctgcaactctggcttcaagcggaaggccggcaccagctccctgacagagtgcgtgctgaacaaggcaacaaatgtggctcactggactaccccatcactgaaatgtattagggattgagtcga

SEQ ID NO: 561 - DNA, 3906 bp, synthetic construct
  misc_feature 1..3906: note = Synthetic sequence
  source 1..3906: mol_type = other DNA
gagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaactagaagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcgccaccatgcggatttccaagccccatctgcgctccatttccatccagtgttacctgtgcctgctgctgaatagccattttctgaccgaagccggcatccacgtgttcatcctgggctgcttttccgccggcctgccaaagaccgaggcaaactgggtgaatgtgatctctgacctgaagaagatcgaggatctgatccagagcatgcacatcgacgccaccctgtacacagagtccgatgtgcacccttcttgcaaggtgacagccatgaagtgtttcctgctggagctgcaggtcatctctctggagagcggcgacgcctctatccacgataccgtggagaacctgatcatcctggccaacaatagcctgagctccaacggcaatgtgacagagtccggctgcaaggagtgtgaggagctggaggagaagaatatcaaagagttcctgcagagtttcgtccatatcgtccagatgtttatcaatacttcctaagaattcacgcgtcgagcatgcatctagggcggccaattccgcccctctcccccccccccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgccacaacccgggatcctctagagtcgacgccaccatgagactggcaggaagacaggtccccgaacagagatcccctccaccacccggactgggcagcgcaagacccggctcacccgccgtgtcctgcggagcagcagcaatggcaccaaggagagccaggggctgtcgcaccctgggcctgcccgccctgttattactgctgctgctgaggccccctgcaacacgcgacgcaagggatcgcctggccgtgctggcaggccggagcagaatctccgagtctttcaaccacgaggtgcagacccacgaggcatgcgtgaggctgaggacaatggagaattgcccccagtgtcaccaccaccggaccagcagacagcaggcaggcatcacatgtccaccccctatgtccgtggagcacgccgacatctgggtgaagagctactccctgtattctcgggagagatacatctgcaacagcggctttaagaggaaggccggcaccagctccctgacagagtgcgtgctgaacaaggccaccaatgtggcccactggaccacaccctctctgaagtgcatccgggatcccgccctggtgcaccagcggcccgcaccacccagcaccgtgaccacagccggcgtgacaccacagccagagtctctgagcccatccggcaaggagcctgcagcatctagcccatcctctaacaataccgcagcaaccacagcagcaatcgtgcctggctcccagctgatgccttctaagagcccatccacaggcaccacagagatcagctcccacgagtctagccacggcaccccttctcagaccacagccaagaattgggagctgacagcatctgccagccaccagcctccaggcgtgtacccacagggccactccgacaccacagtggccatctccacctctacagtgctgctgtgcggcctgagcgccgtgtccctgctggcctgttatctgaagtccagacagaccccccctctggcctctgtggagatggaggccatggaggccctgcccgtcacctggggaacttcatcaagagacgaggacctggaaaactgtagtcaccatctgtaagtcga

SEQ ID NO: 562 - DNA, 3487 bp, synthetic construct
  misc_feature 1..3487: note = Synthetic sequence
  source 1..3487: mol_type = other DNA
gagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaactagaagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcgccaccatgcggatttccaaacctcacctgcgatctatctccatccagtgctatctgtgcctgctgctgaactctcatttcctgaccgaagccggcatccacgtgttcatcctgggctgctttagcgccggcctgccaaagaccgaggcaaactgggtgaatgtgatctctgacctgaagaagatcgaggatctgatccagagcatgcacatcgacgccaccctgtacacagagtccgatgtgcacccttcttgcaaggtgacagccatgaagtgtttcctgctggagctgcaggtcatctctctggagagcggcgacgcctccatccacgataccgtggagaacctgatcatcctggccaacaatagcctgagctccaacggcaatgtgacagagtccggctgcaaggagtgtgaggagctggaggagaagaacatcaaggagttcctgcagtcttttgtgcacatcgtgcagatgtttatcaataccagcggaggaggaggatccggcggaggaggctctggcggcggcggcagctgtggcggcaaggtgtccgccctgaaggagaaggtgtctgccctgaaggaaaaagtgtccgctctgaaggaaaaggtgtccgcactgaaagaaaaggtctccgccctgaaggaataagaattcacgcgtcgagcatgcatctagggcggccaattccgcccctctcccccccccccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgccacaacccgggatcctctagagtcgacgccaccatggcccctagaagagcccgaggatgtcgcacactgggactgcctgccctgctgctgctgctgctgctgagaccacctgctacaaggggcatcacctgcccacctccaatgagcgtggagcacgcagacatctgggtgaagtcttacagcctgtattctcgggagagatacatctgcaacagcggcttcaagaggaaggccggcaccagctccctgacagagtgcgtgctgaacaaggccacaaatgtggcccactggaccacaccctccctgaagtgtatccgggatggaggaggaggaagcggaggaggaggatccggcggcggcggctccgaggtgtctgccctggagaaggaggtgtctgccctggaaaaagaagtctcagccctggaaaaggaggtgtcagccctggaaaaagaggtgtccgcactggaaaaataagtcgaagtcgggacctttttctccacaggcgtgacctttttattcagc

SEQ ID NO: 563 - DNA, 4215 bp, synthetic construct
  misc_feature 1..4215: note = Synthetic sequence
  source 1..4215: mol_type = other DNA
gagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaactagaagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcgccaccatgcggatttccaaacctcacctgcgatctatctccatccagtgctatctgtgcctgctgctgaactctcatttcctgaccgaagccggcatccacgtgttcatcctgggctgctttagcgccggcctgccaaagaccgaggcaaactgggtgaatgtgatctctgacctgaagaagatcgaggatctgatccagagcatgcacatcgacgccaccctgtacacagagtccgatgtgcacccttcttgcaaggtgacagccatgaagtgtttcctgctggagctgcaggtcatctctctggagagcggcgacgcctccatccacgataccgtggagaacctgatcatcctggccaacaatagcctgagctccaacggcaatgtgacagagtccggctgcaaggagtgtgaggagctggaggagaagaacatcaaggagttcctgcagtcttttgtgcacatcgtgcagatgtttatcaataccagcggaggaggaggatccggcggaggaggctctggcggcggcggcagctgtggcggcaaggtgtccgccctgaaggagaaggtgtctgccctgaaggaaaaagtgtccgctctgaaggaaaaggtgtccgcactgaaagaaaaggtctccgccctgaaggaataagaattcacgcgtcgagcatgcatctagggcggccaattccgcccctctcccccccccccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgccacaacccgggatcctctagagtcgacgccaccatgcggctggctggaaggcaggtccccgagcagagatcccctcctcctcctgggctgggcagtgcaagacccggcagtcccgctgtgtcctgcggagcagcagcaatggcaccaaggagagccaggggctgtcgcaccctgggcctgcccgccctgttattactgctgctgctgaggccccctgcaacacgcgacgcaagggatcgcctggccgtgctggcaggccggagcagaatcagcgagtccttcaaccacgaggtgcagacccacgaggcatgcgtgaggctgaggacaatggagaattgcccccagtgtcaccaccaccggaccagcagacagcaggcaggcatcacatgtccaccccctatgtccgtggagcacgccgacatctgggtgaagtcttacagcctgtattctcgggagagatacatctgcaacagcggctttaagcggaaggccggcaccagctccctgacagagtgcgtgctgaacaaggccaccaatgtggcccactggaccacaccctctctgaagtgcatccgggatcccgccctggtgcaccagcggcccgcaccacccagcaccgtgaccacagcaggagtgacaccacagccagagtccctgtctccaagcggcaaggagcctgcagcatctagcccatcctctaacaataccgcagcaaccacagcagcaatcgtgcctggctcccagctgatgccttccaagtctccaagcaccggcaccacagagatcagctcccacgagtctagccacggcaccccttctcagaccacagccaagaactgggagctgacagcatccgcctctcaccagcctccaggcgtgtacccacagggccactccgacaccacagtggccatcagcacctccacagtgctgctgtgcggcctgtctgccgtgagcctgctggcctgttatctgaagtccaggcagaccccacctctggcatctgtggagatggaggcaatggaggccctgccagtgacctggggcacatcctctcgcgacgaggatctggagaattgttcccaccacctgggaggaggaggaagcggaggaggaggctccggcggcggcggctccgaggtgtctgccctggaaaaagaggtgagcgccctggaaaaagaagtgtcagccctggagaaggaggtgtcagcactggaaaaagaggtgtccgcactggaaaaataagtcga

SEQ ID NO: 564 - DNA, 3425 bp, synthetic construct
  misc_feature 1..3425: note = Synthetic sequence
  source 1..3425: mol_type = other DNA
acattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactagtccagtgtggtggaattcgccaccatgtgccctcagaaactgactatctcatggttcgcaatcgtgctgctggtctcacccctgatggctatgtgggaactggaaaaggatgtgtacgtggtggaggtggactggacacccgatgcccctggcgagacagtgaacctgacatgcgacacccccgaggaggacgacatcacctggacatccgatcagcggcacggcgtgatcggctctggcaagaccctgacaatcaccgtgaaggagttcctggacgccggccagtacacatgtcacaagggcggcgagacactgagccactcccacctgctgctgcacaagaaggagaacggcatctggtccacagagatcctgaagaacttcaagaataagacctttctgaagtgcgaggcccccaattattctggccggttcacctgtagctggctggtgcagagaaacatggacctgaagtttaatatcaagagctcctctagctcccctgatagcagggcagtgacatgcggaatggcatctctgagcgccgagaaggtgaccctggaccagagagattacgagaagtattccgtgtcttgccaggaggatgtgacatgtcccaccgccgaggagacactgcctatcgagctggccctggaggcaaggcagcagaacaagtacgagaattatagcacctccttctttatcagagacatcatcaagccagatccccctaagaacctgcagatgaagcccctgaagaacagccaggtcgaggtgagctgggagtaccctgactcttggagcacaccacactcttatttcagcctgaagttctttgtgaggatccagcgcaagaaggagaagatgaaggagacagaggagggctgcaaccagaagggcgcctttctggtggagaagacatccaccgaggtgcagtgcaagggaggaaacgtgtgcgtgcaggcacaggatagatactataattctagctgctctaagtgggcctgcgtgccctgtagggtgcgctccggaggcggcggctctggaggaggaggaagcggaggaggaggaagcatgtgccagtccaggtacctgctgttcctggccaccctggccctgctgaaccacctgtccctggcacgcgtgatcccagtgtctggaccagcccggtgcctgtcccagtctagaaatctgctgaagaccacagacgatatggtgaagacagccagggagaagctgaagcactattcctgtaccgccgaggacatcgatcacgaggacatcacacgcgatcagacatccaccctgaagacctgcctgcctctggagctgcacaagaacgagtcttgtctggccacacgggagacaagctctaccacaagaggcagctgcctgccaccccagaagacatccctgatgatgaccctgtgcctgggcagcatctacgaggacctgaagatgtatcagacagagtttcaggccatcaatgccgccctgcagaaccacaatcaccagcagatcatcctggacaagggcatgctggtggccatcgatgagctgatgcagtccctgaaccacaatggcgagacactgaggcagaagcctccagtgggcgaggccgatccttaccgcgtgaagatgaagctgtgcatcctgctgcacgccttcagcacaagggtggtgaccatcaaccgcgtgatgggctatctgagctccgccggaggaggaggatccggcggaggaggctctggcggcggcggcagcggctccggcgccaccaacttttctctgctgaagcaggcaggcgacgtggaggagaatccaggacccatgaggatcagcaagccacacctgcgctctatcagcatccagtgctacctgtgcctgctgctgaatagccacttcctgacagaggccggcatccacgtgttcatcctgggctgtttttccgccggcctgccaaagaccgaggccaactgggtgaatgtgatctctgacctgaagaagatcgaggatctgatccagagcatgcacatcgacgccacactgtataccgagagcgatgtgcacccctcctgcaaggtgaccgccatgaagtgttttctgctggagctgcaggtcatcagcctggagtctggcgacgccagcatccacgatacagtggagaacctgatcatcctggccaacaatagcctgtctagcaacggcaatgtgaccgagtccggctgcaaggagtgtgaggagctggaggagaagaacatcaaggagttcctgcagtcctttgtgcacatcgtgcagatgttcatcaatacatccggcggcggcggctccggcggcggagggagcggaggaggcggctccggctctggcgccaccaactttagcctgctgaagcaggccggcgacgtggaagaaaatcctggaccaatggcacctaggagagcaaggggatgcagaaccctgggcctgccagccctgttattactgctgctgctgaggccccctgcaacaaggggaatcacctgtccaccccctatgagcgtggagcacgccgacatctgggtgaagagctactccctgtattctcgggagagatacatctgcaatagcggcttcaagcggaaggccggcacatcctctctgaccgagtgcgtgctgaacaaagcaacaaacgtggctcattggacaacaccatccctgaagtgcatccgcgactaagat

SEQ ID NO: 565 - DNA, 3912 bp, synthetic construct
  misc_feature 1..3912: note = Synthetic sequence
  source 1..3912: mol_type = other DNA
acattgattattgactagttattaatagtaatcaattacggggtcattagttcatagcccatatatggagttccccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactagtccagtgtggtggaattcgccaccatgtgccctcagaagctgactatctcttggtttgctattgtgctgctggtctcacccctgatggctatgtgggaactggaaaaggatgtgtacgtggtggaggtggactggacaccagatgcccccggcgagacagtgaacctgacatgcgacacccccgaggaggacgacatcacctggacatctgatcagaggcacggcgtgatcggaagcggcaagaccctgacaatcaccgtgaaggagttcctggacgccggccagtacacatgtcacaagggcggcgagacactgtctcacagccacctgctgctgcacaagaaggagaacggcatctggagcacagagatcctgaagaacttcaagaataagacctttctgaagtgcgaggccccaaattattccggcaggttcacctgttcttggctggtgcagcgcaacatggacctgaagtttaatatcaagagctcctctagctcccccgattccagggcagtgacatgcggaatggcatccctgtctgccgagaaggtgaccctggaccagcgcgattacgagaagtatagcgtgtcctgccaggaggatgtgacatgtcctaccgccgaggagacactgccaatcgagctggccctggaggccaggcagcagaacaagtacgagaattattctaccagcttctttatccgcgacatcatcaagccagatccccctaagaacctgcagatgaagcccctgaagaacagccaggtcgaggtgtcttgggagtacccagactcctggtctacaccccactcttatttcagcctgaagttctttgtgaggatccagcgcaagaaggagaagatgaaggagacagaggagggctgcaaccagaagggcgcctttctggtggagaagacatccaccgaggtgcagtgcaagggaggaaacgtgtgcgtgcaggcacaggatagatactataattctagctgcagcaagtgggcctgcgtgccttgtagggtgcgcagcggaggaggaggatccggaggaggcggctctggaggaggaggatctatgtgccagagccggtacctgctgttcctggccaccctggccctgctgaaccacctgagcctggcaagagtgatccccgtgagcggaccagcaaggtgcctgagccagtccagaaatctgctgaagaccacagacgatatggtgaagacagcccgggagaagctgaagcactatagctgtaccgccgaggacatcgatcacgaggacatcacaagagatcagacatctaccctgaagacctgcctgcccctggagctgcacaagaacgagagctgtctggccacacgggagacaagctctaccacaagaggctcttgcctgccaccccagaagacaagcctgatgatgaccctgtgcctgggcagcatctacgaggacctgaagatgtatcagaccgagtttcaggccatcaatgccgccctgcagaaccacaatcaccagcagatcatcctggacaagggcatgctggtggccatcgatgagctgatgcagagcctgaaccacaatggcgagacactgaggcagaagcctccagtgggagaggcagatccatacagagtgaagatgaagctgtgcatcctgctgcacgccttctccacaagggtggtgaccatcaaccgcgtgatgggctatctgagctccgccggaggaggaggaagcggcggaggaggcagcggcggcggcggctctggcagcggcgccaccaactttagcctgctgaagcaggcaggcgacgtggaggagaatccaggacccatgaggatctccaagccccacctgcgctccatctctatccagtgctacctgtgcctgctgctgaacagccacttcctgacagaggccggcatccacgtgttcatcctgggctgtttttctgccggcctgcctaagaccgaggccaactgggtgaatgtgatcagcgacctgaagaagatcgaggatctgatccagtccatgcacatcgacgccacactgtataccgagtctgatgtgcacccaagctgcaaggtgaccgccatgaagtgttttctgctggagctgcaggtcatcagcctggagtctggcgacgccagcatccacgatacagtggagaacctgatcatcctggccaacaattccctgtctagcaacggcaatgtgaccgagtctggctgcaaggagtgtgaggagctggaggagaagaacatcaaggagttcctgcagagctttgtgcacatcgtgcagatgttcatcaatacaagcggaggcggaggatctggcggcggcggcagtggcggaggaggaagcggctccggcgccaccaacttttccctgctgaagcaggccggcgacgtggaagaaaatcctggaccaatggcacctaggagagcaaggggatgcagaaccctgggcctgccagccctgttattactgctgctgctgaggccccctgcaacaaggggaatcacctgtccaccccctatgagcgtggagcacgccgacatctgggtgaagtcttacagcctgtattcccgggagagatacatctgcaactctggcttcaagaggaaggccggcacatcctctctgaccgagtgcgtgctgaacaaggccaccaatgtggcccactggaccacaccctctctgaagtgcatccgggaccccgccctggtgcaccagcggcccgcaccacccagcacagtgaccacagcaggagtgaccccacagcctgagtccctgtctccaagcggcaaggagccagcagcaagctccccttctagcaacaatacagcagcaaccacagcagcaatcgtgcctggctcccagctgatgccctccaagtctcctagcaccggcaccacagagatctcctctcacgagagctcccacggcacacccagccagaccacagccaagaattgggagctgaccgcatccgcctctcaccagcctccaggcgtgtaccctcagggccactccgataccacagtggccatcagcacatccaccgtgctgctgtgcggcctgagcgccgtgtccctgctggcctgttatctgaagagcaggcagaccccacctctggcatccgtggagatggaggccatggaggccctgcccgtcacctgggggacatcatcacgggacgaagacctggagaactgctcacatcatctgtaagat

SEQ ID NO: 566 - DNA, 3808 bp, synthetic construct
  misc_feature 1..3808: note = Synthetic sequence
  source 1..3808: mol_type = other DNA
acattgattattgactagttattaatagtaatcaattacggggtcattagttcatagcccatatatggagttccgcgttacataacttacggtaaatggctgtaactaataactgatcaataattatcattagttaatgccccagtaatcaagtatcgggtatatacctcaaggccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactagtccagtgtggtggaattcgccaccatgtgccctcagaaactgacaatctcctggttcgctatcgtcctgctggtctctcctctgatggctatgtgggaactggaaaaagatgtgtacgtggtggaggtggactggacacccgatgcccctggcgagacagtgaacctgacatgcgacacccccgaggaggacgacatcacctggacatccgatcagaggcacggcgtgatcggctctggcaagaccctgacaatcaccgtgaaggagttcctggacgccggccagtacacatgtcacaagggcggcgagacactgagccactcccacctgctgctgcacaagaaggagaacggcatctggtccacagagatcctgaagaacttcaagaataagacctttctgaagtgcgaggcccccaattatagcggccggttcacctgttcctggctggtgcagagaaacatggacctgaagtttaatatcaagagctcctctagctcccctgatagcagggcagtgacatgcggaatggcatctctgagcgccgagaaggtgaccctggaccagagagattacgagaagtattccgtgtcttgccaggaggatgtgacatgtcccaccgccgaggagacactgcctatcgagctggccctggaggcaaggcagcagaacaagtacgagaattatagcacctccttctttatcagagacatcatcaagccagatccccctaagaacctgcagatgaagcccctgaagaacagccaggtcgaggtgtcttgggagtaccctgactcttggagcacaccacactcttatttcagcctgaagttctttgtgaggatccagcgcaagaaggagaagatgaaggagacagaggagggctgtaaccagaagggcgcctttctggtggagaagacatccaccgaggtgcagtgcaagggaggaaacgtgtgcgtgcaggcacaggatcggtactataattctagctgctctaagtgggcctgcgtgccctgtagggtgcgcagcggaggaggaggatccggaggaggcggctctggaggaggaggatctatgtgccagagcaggtacctgctgttcctggccaccctggccctgctgaaccacctgtccctggcacgcgtgatcccagtgtctggaccagcccggtgcctgtcccagtctagaaatctgctgaagaccacagacgatatggtgaagacagccagggagaagctgaagcactatagctgtaccgccgaggacatcgatcacgaggacatcacacgcgatcagacatccaccctgaagacctgcctgcctctggagctgcacaagaacgagtcttgtctggccacacgggagacaagctctaccacaagaggcagctgcctgccaccccagaagacatccctgatgatgaccctgtgcctgggcagcatctacgaggacctgaagatgtatcagacagagtttcaggccatcaatgccgccctgcagaaccacaatcaccagcagatcatcctggacaagggcatgctggtggccatcgatgagctgatgcagtccctgaaccacaatggcgagacactgaggcagaagcctccagtgggcgaggccgatccttaccgcgtgaagatgaagctgtgcatcctgctgcacgccttcagcacaagggtggtgaccatcaaccgcgtgatgggctatctgagctccgccggaggaggaggaagcggcggaggaggcagcggcggcggcggcagcggctccggcgccaccaacttttctctgctgaagcaggcaggcgacgtggaggagaatccaggacccatgaggatcagcaagccacacctgcgctctatcagcatccagtgctacctgtgcctgctgctgaatagccacttcctgacagaggccggcatccacgtgttcatcctgggctgtttttccgccggcctgccaaagaccgaggccaactgggtgaatgtgatctctgacctgaagaagatcgaggatctgatccagagcatgcacatcgacgccacactgtataccgagtctgatgtgcaccccagctgcaaggtgaccgccatgaagtgttttctgctggagctgcaggtcatcagcctggagtctggcgacgcctccatccacgatacagtggagaacctgatcatcctggccaacaatagcctgtctagcaacggcaatgtgaccgagtccggctgcaaggagtgtgaggagctggaggagaagaacatcaaggagttcctgcagagctttgtgcacatcgtgcagatgttcatcaatacctccggcggcggaggatctggcggcggcggctccggcgggggaggatcctgcggcggcaaggtgtctgccctgaaggagaaggtgagcgccctgaaggaaaaggtgtccgccctgaaggagaaggtgtctgccctgaaggaaaaggtgagcgccctgaaggagggcggcggaggatctgggggcggcggctccgggggaggaggatccggctctggcgccacaaacttctccctgctgaagcaggccggcgacgtggaagaaaatcctggaccaatggcacctaggagagcaaggggatgcagaaccctgggcctgccagccctgttattactgctgctgctgaggccccctgcaacaaggggaatcacctgtccaccccctatgagcgtggagcacgccgacatctgggtgaagagctactccctgtattctcgggagagatacatctgcaatagcggctttaagcggaaggccggcacatcctctctgaccgagtgcgtgctgaacaaggccaccaatgtggcccactggaccacaccctccctgaagtgcatcagagatggcggcggcggatccggaggaggcgggtctggcggcggcggcagcgaggtgtccgccctggagaaggaggtgagcgccctggaaaaagaggtgagtgctctggaaaaggaagtgtctgccctggaaaaggaagtgtctgccctggaaaaatgagat

SEQ ID NO: 567 - DNA, 4247 bp, synthetic construct
  misc_feature 1..4247: note = Synthetic sequence
  source 1..4247: mol_type = other DNA
acattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactagtccagtgtggtggaattcgccaccatgtgcccccagaaactgacaatctcttggttcgcaatcgtgctgctggtctcacccctgatggctatgtgggagctggaaaaggatgtgtacgtggtggaggtggactggacaccagatgcccccggcgagacagtgaacctgacatgcgacacccccgaggaggacgacatcacctggacatccgatcagcggcacggcgtgatcggctctggcaagaccctgacaatcaccgtgaaggagttcctggacgccggccagtacacatgtcacaagggcggcgagacactgtcccactctcacctgctgctgcacaagaaggagaacggcatctggagcacagagatcctgaagaacttcaagaataagacctttctgaagtgcgaggccccaaattattccggcaggttcacctgttcttggctggtgcagcgcaacatggacctgaagtttaatatcaagagctcctctagctcccccgattccagggcagtgacatgcggaatggcaagcctgtccgccgagaaggtgaccctggaccagagggattacgagaagtattctgtgagctgccaggaggatgtgacatgtcctaccgccgaggagacactgccaatcgagctggccctggaggccaggcagcagaacaagtacgagaattattccacctctttctttatccgcgacatcatcaagccagatccccctaagaacctgcagatgaagcccctgaagaacagccaggtcgaggtgagctgggagtacccagacagctggtccacaccccactcctatttctctctgaagttctttgtgaggatccagcgcaagaaggagaagatgaaggagacagaggagggctgtaaccagaagggcgcctttctggtggagaagacatccaccgaggtgcagtgcaagggaggaaacgtgtgcgtgcaggcacaggatagatactataattctagctgcagcaagtgggcctgcgtgccttgtagggtgcgctccggaggcggcggctctggaggaggaggaagcggaggaggaggaagcatgtgccagtcccggtacctgctgttcctggccaccctggccctgctgaaccacctgtctctggccagagtgatccctgtgagcggaccagcaaggtgcctgtctcagagcagaaatctgctgaagaccacagacgatatggtgaagacagcccgggagaagctgaagcactatagctgtaccgccgaggacatcgatcacgaggacatcacaagagatcagacatccaccctgaagacctgcctgcccctggagctgcacaagaacgagtcttgtctggccacacgggagacaagctctaccacaagaggctcctgcctgccaccccagaagacatctctgatgatgaccctgtgcctgggcagcatctacgaggacctgaagatgtatcagaccgagtttcaggccatcaatgccgccctgcagaaccacaatcaccagcagatcatcctggacaagggcatgctggtggccatcgatgagctgatgcagagcctgaaccacaatggcgagacactgaggcagaagcctccagtgggagaggcagatccatacagagtgaagatgaagctgtgcatcctgctgcacgccttctccacaagggtggtgaccatcaaccgcgtgatgggctatctgagctccgccggaggaggaggatccggcggaggaggctctggcggcggcggctccggctctggcgccaccaacttttctctgctgaagcaggcaggcgacgtggaggagaatccaggacccatgaggatcagcaagccccacctgcgcagcatctccatccagtgctacctgtgcctgctgctgaatagccacttcctgacagaggccggcatccacgtgttcatcctgggctgtttttccgccggcctgcctaagaccgaggccaactgggtgaatgtgatctctgacctgaagaagatcgaggatctgatccagagcatgcacatcgacgccacactgtataccgagagcgatgtgcacccatcctgcaaggtgaccgccatgaagtgttttctgctggagctgcaggtcatcagcctggagtccggcgacgcaagcatccacgatacagtggagaacctgatcatcctggccaacaatagcctgtctagcaacggcaatgtgaccgagtccggctgcaaggagtgtgaggagctggaggagaagaacatcaaggagttcctgcagtcctttgtgcacatcgtgcagatgttcatcaatacttccggcggcggcggcagtggcggaggaggaagcggaggcggcggctcttgcggcggcaaggtgagcgccctgaaggagaaggtgtccgccctgaaggaaaaggtgtctgccctgaaggagaaggtgagcgccctgaaggaaaaggtgtccgccctgaaggaggggggcggcggcagcggcggaggagggagcgggggaggcggctctggcagcggcgccacaaacttcagcctgctgaagcaggccggcgacgtggaagaaaatcctggaccaatggcacctaggagagcaaggggatgcagaaccctgggcctgccagccctgttattactgctgctgctgaggccccctgcaacaaggggaatcacctgtccaccccctatgagcgtggagcacgccgacatctgggtgaagtcctactctctgtatagccgggagagatacatctgcaattccggctttaagaggaaggccggcacatcctctctgaccgagtgcgtgctgaacaaggccaccaatgtggcccactggaccacaccatccctgaagtgcatccgggaccccgccctggtgcaccagcggcccgcaccaccatctacagtgaccacagcaggagtgaccccacagcctgagagcctgtccccatctggcaaggagccagcagcaagctccccttctagcaacaatacagcagcaaccacagcagcaatcgtgcctggctcccagctgatgcccagcaagtccccttctaccggcaccacagagatctcctctcacgagagctcccacggcacacccagccagaccacagccaagaactgggagctgaccgcaagcgcctcccaccagcctccaggcgtgtaccctcagggccactccgataccacagtggccatctctacaagcaccgtgctgctgtgcggcctgtctgccgtgagcctgctggcctgttatctgaagtctaggcagaccccacctctggcaagcgtggagatggaggcaatggaggccctgccagtgacatggggcacctctagccgcgacgaggatctggagaattgcagccaccacctgggaggaggaggatcaggcggcggcgggtccggcggcggcggatccgaggtgtctgccctggagaaggaggtgtccgccctggaaaaagaagtgtcagccctggagaaagaagtgtcagccctggaaaaagaggtgtctgccctggaaaaataagat

SEQ ID NO: 568 - protein, 897 aa, synthetic construct
  REGION 1..897: note = Synthetic sequence
MCPQKLTISWFAIVLLVSPLMAMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGGGGSGGGGSGGGGSMCQSRYLLFLATLALLNHLSLARVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAGGGGSGGGGSGGGGSGSGATNFSLLKQAGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGSGGGGSGGGGSGSGATNFSLLKQAGDVEENPGPMAPRRARGCRTLGLPALLLLLLLRPPATRGITCPPPMSVEHADIWVKSYSLYSRERYICNSGFKRKAGTSSLTECVLNKATNVAHWTTPSLKCIRD

SEQ ID NO: 569 - protein, 1068 aa, synthetic construct
  REGION 1..1068: note = Synthetic sequence
MCPQKLTISWFAIVLLVSPLMAMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGGGGSGGGGSGGGGSMCQSRYLLFLATLALLNHLSLARVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAGGGGSGGGGSGGGGSGSGATNFSLLKQAGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGSGGGGSGGGGSGSGATNFSLLKQAGDVEENPGPMAPRRARGCRTLGLPALLLLLLLRPPATRGITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 570 - protein, 1000 aa, synthetic construct
  REGION 1..1000: note = Synthetic sequence
MCPQKLTISWFAIVLLVSPLMAMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGGGGSGGGGSGGGGSMCQSRYLLFLATLALLNHLSLARVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAGGGGSGGGGSGGGGSGSGATNFSLLKQAGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGSGGGGSGGGGSCGGKVSALKEKVSALKEKVSALKEKVSALKEKVSALKEGGGGSGGGGSGGGGSGSGATNFSLLKQAGDVEENPGPMAPRRARGCRTLGLPALLLLLLLRPPATRGITCPPPMSVEHADIWVKSYSLYSRERYICNSGFKRKAGTSSLTECVLNKATNVAHWTTPSLKCIRDGGGGSGGGGSGGGGSEVSALEKEVSALEKEVSALEKEVSALEKEVSALEK

SEQ ID NO: 571 - protein, 1171 aa, synthetic construct
  REGION 1..1171: note = Synthetic sequence
MCPQKLTISWFAIVLLVSPLMAMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGGGGSGGGGSGGGGSMCQSRYLLFLATLALLNHLSLARVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAGGGGSGGGGSGGGGSGSGATNFSLLKQAGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGSGGGGSGGGGSCGGKVSALKEKVSALKEKVSALKEKVSALKEKVSALKEGGGGSGGGGSGGGGSGSGATNFSLLKQAGDVEENPGPMAPRRARGCRTLGLPALLLLLLLRPPATRGITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHLGGGGSGGGGSGGGGSEVSALEKEVSALEKEVSALEKEVSALEKEVSALEK

SEQ ID NO: 572 - DNA, 1230 bp, Herpes Simplex Virus
  source 1..1230: mol_type = genomic DNA
gtgacgattcccccaatggccgcgcgtcccaggggaggcaggcccaccgcggggcggccccgtccccggggaccaacccggcgcccccaaagaatatcattagcatgcacggcccggcccccgatttgggggcccaacccggtgtcccccaaagaaccccattagcatgcccctcccgccgacgcaacaggggcttggcctgcgtcggtgccccggggcttcccgccttcccgaagaaactcattaccatacccggaaccccaggggaccaatgcgggttcattgagcgacccgcgggccaatgcgcgaggggccgtgtgttccgccaaaaaagcaattagcataacccggaaccccaggggagtggttacgcgcggcgcgggaggcggggaataccggggttgcccattaagggccgcgggaattgccggaagcgggaagggcggccggggccgcccattagttacctgggactgtgcggttgggacggcgcccgtgggcccgggcggccgggggcggcgggggccgcgatggcggcggcggcgggccatggagacagagagcgtgccggggtggtagagtttgacaggcaagcatgtgcgtgcagaggcgagtagtgcttgcctgtctaactcgctagtctcggccgcggggggcccgggctgcccgccgccgccgctttaaagggccgcgcgcgacccccggggggtgtgttttggggggggcccgttttcggggtctggccgctcctccccccgctcctccccccgctcctccccccgctcctccccccgctcctccccccgctcctccccccgctcctccccccgctcctccccccgctcctccccccgctcctccccccgctcctccccccgctcctccccccgctcctccccccgctcctccccccgctcctccccccgctcctccccccgctcctccccccgctcctccccccgctcccgcggccccgccccccacgcccgccgcgcgcgcgcacgccgcccggaccgccgcccgccttttttgcgcgcgcgcgcgcccgcggggggcccgggctgccacaggtgaaaccaacagagcacggcgcactccgcacgtcacacgtcacgtcatccaccacacctgcccaacaacacaactcacagcgacaactcaccgcgcaacaactcctgttcctcatccacacgtcaccgcgcacctcccgctcctccagacgtaccccgg

SEQ ID NO: 573 - DNA, 3185 bp, Herpes Simplex Virus
  source 1..3185: mol_type = genomic DNA
atgctcgccgtccgttccctgcagcacctctcaaccgtcgtcttgataacggcgtacggcctcgtgctcgtgtggtacaccgtcttcggtgccagtccgctgcaccgatgtatttacgcggtacgccccaccggcaccaacaacgacaccgccctcgtgtggatgaaaatgaaccagaccctattgtttctgggggccccgacgcacccccccaacgggggctggcgcaaccacgcccatatctgctacgccaatcttatcgcgggtagggtcgtgcccttccaggtcccacctgacgccatgaatcgtcggatcatgaacgtccacgaggcagttaactgtctggagaccctatggtacacacgggtgcgtctggtggtcgtagggtggttcctgtatctggcgttcgtcgccctccaccaacgccgatgtatgtttggcgtcgtgagtcccgcccacaagatggtggccccggccacctacctcttgaactacgcaggccgcatcgtatcgagcgtgttcctgcagtacccctacacgaaaattacccgcctgctctgcgagctgtcggtccagcggcaaaacctggttcagttgtttgagacggacccggtcaccttcttgtaccaccgccccgccatcggggtcatcgtaggctgcgagttgatgctacgctttgtggccgtgggtctcatcgtcggcaccgctttcatatcccggggggcatgtgcgatcacataccccctgtttctgaccatcaccacctggtgttttgtctccaccatcggcctgacagagctgtattgtattctgcggcggggcccggcccccaagaacgcagacaaggccgccgccccggggcgatccaaggggctgtcgggcgtctgcgggcgctgctgttccatcatcctctcgggcatcgcagtgcgattgtgttatatcgccgtggtggccggggtggtgctcgtggcgcttcactacgagcaggagatccagaggcgcctgtttgatgtatgacgtcacatccaggccggcggaaaccgtaacggcatatgcaaattggaaactgtcctgtcttggggcccacccacccgacgcgtcatatgcaaatgaaaatcggtcccccgaggccacgtgtagcctggatcccaacgaccccgcccatgggtcccaattggccgtcccgttaccaagaccaacccagccagcatatccacccccgcccgggtccccgcggaagcggaacggggtatgtgatatgctaattaaatacgggaatttccggggactttccgggaatttccggggactttccgggaatttccctacagaggtgcatattaacagagcttttgtcctggagaatgccacgtacttatggtgtctgattggtccttgtctgtgccggaggtggggcgggggccccgcccggggggcggaacgaggaggggtttgggagagccggccccggcaccacgggtataaggacatccaccacccggccggtggtggtgtgcagccgtgttccaaccacggtcacgcttcggtgcctctccccgattcgggcccggtcgctcgctaccggtgcgccaccaccagaggccatatccgacaccccagccccgacggcagccgacagcccggtcatggcgactgacattgatatgctaattgacctcggcctggacctctccgacagcgatctggacgaggacccccccgagccggcggagagccgccgcgacgacctggaatcggacagcagcggggagtgttcctcgtcggacgaggacatggaagacccccacggagaggacggaccggagccgatactcgacgccgctcgcccggcggtccgcccgtctcgtccagaagaccccggcgtacccagcacccagacgcctcgtccgacggagcggcagggccccaacgatcctcaaccagcgccccacagtgtgtggtcgcgcctcggggcccggcgaccgtcttgctcccccgagcagcacgggggcaaggtggcccgcctccaacccccaccgaccaaagcccagcctgcccgcggcggacgccgtgggcgtcgcaggggtcggggtcgcggtggtcccggggctgccgatggtttgtcggacccccgccggcgtgcccccagaaccaatcgcaaccctgggggaccccgccccggggcggggtggacggacggccccggcgccccccatggcgaggcgtggcgcggcagtgagcagcccgacccacccggaggccagcggacacggggcgtgcgccaagcaccccccccgctaatgacgctggcgattgcccccccgcccgcggacccccgcgccccggccccggagcgaaaggcgcccgccgccgacaccatcgacgccaccacgcggttggtcctgcgctccatctccgagcgcgcggcggtcgaccgcatcagcgagagctttggccgcagcgcacaggtcatgcacgacccctttggggggcagccgtttcccgccgcgaatagcccctgggccccggtgctggcgggccaaggagggccctttgacgccgagaccagacgggtctcctgggaaaccttggtcgcccacggcccgagcctctatcgcacttttgccggcaatcctcgggccgcatcgaccgccaaggccatgcgcgactgcgtgctgcgccaagaaaatttcatcgaggcgctggcctccgccgacgagacgctggcgtggtgcaagatgtgcatccaccacaacctgccgctgcgcccccaggaccccattatcgggacggccgcggctgtgctggataacctcgccacgcgcctgcggccctttctccagtgctacctgaaggcgcgaggcctgtgcggcctggacgaactgtgttcgcggcggcgtctggcggacattaaggacattgcatccttcgtgtttgtcattctggccaggctcgccaaccgcgtcgagcgtggcgtcgcggagatcgactacgcgacccttggtgtcggggtcggagagaagatgcatttctacctccccggggcctgcatggcgggcctgatcgaaatcctagacacgcaccgccaggagtgttcgagtcgtgtctgcgagttgacggccagtcacatcgtcgcccccccgtacgtgcacggcaaatatttttattgcaactccctgttttag

SEQ ID NO: 574 - DNA, 3670 bp, Herpes Simplex Virus
  source 1..3670: mol_type = genomic DNA
atgtcgggcgtcgggggagagggagttccctctgcgcttgcgattctagcctcgtggggctggacgttcgacacgccaaaccacgagtcggggatatcgccagatacgactcccgcagattccattcggggtgccgctgtggcctcacctgaccaacctttacacgggggcccggaacgggaggccacagcgccgtctttctccccaacgcgcgcggatgacggcccgccctgtaccgacgggccctacgtgacgtttgataccctgtttatggtgtcgtcgatcgacgaattagggcgtcgccagctcacggacaccatccgcaaggacctgcggttgtcgctggccaagtttagcattgcgtgcaccaagacctcctcgttttcgggaaacgccccgcgccaccacagacgcggggcgttccagcgcggcacgcgggcgccgcgcagcaacaaaagcctccagatgtttgtgttgtgcaaacgcgcccacgccgctcgagtgcgagagcagcttcgggtcgttattcagtcccgcaagccgcgcaagtattacacgcgatcttcggacgggcggctctgccccgccgtccccgtgttcgtccacgagttcgtctcgtccgagccaatgcgcctccaccgagataacgtcatgctggcctcgggggccgagtaaccgcccccccgcgccaccctcactgcccgtcgcgcgtgtttgatgttaataaataacgcataaatttggctggttgtttgttgtctttaatggaccgcccgcagggggggtggcatttcagtgtcgggtgacgagcgcgatccggccgggagaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagctagcatgtacaggatgcaactcctgtcttgcattgcactaagtcttgcacttgtcacgaattcgatatcgacagcccacccctctcctagcccaagatccgccggccagttcgccatggttagatctcccccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctccgggtaaatgagtggatccaccggatctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcctaggaccccaaaagtttgtctgcgtattccagggcggggctcagttgaatctcccgcagcacctctaccagcaggtccgcggtgggctggagaaactcggccgtcccggggcaggcggtcgtcgggggtggaggcgcggcgcccaccccgtgtgccgcgcctggcgtctcctctgggggcgacccgtaaatggttgcagtgatgtaaatggtgtccgcggtccagaccacggtcaaaatgccggccgtggcgctccgggcgctttcgccgcgcgaggagctgacccaggagtcgaacggatacgcgtacatatgggcgtcccacccgcgttcgagcttctggttgctgtcccggcctataaagcggtaggcacaaaattcggcgcgacagtcgataatcaccaacagcccaatgggggtgtgctggataacaacgcctccgcgcggcaggcggtcctggcgctcccggccccgtaccatgatcgcgcgggtgccgtactcaaaaacatgcaccacctgcgcggcgtcgggcagtgcgctggtcagcgaggccctggcgtggcataggctatacgcgatggtcgtctgtggattggacat

SEQ ID NO: 575 - DNA, 6903 bp, Herpes Simplex Virus
  source 1..6903: mol_type = genomic DNA
ttagaggcgccgggagtggggtcgtcggcgtctgctttttgtggcggcgtcccgtcgcggggtggggtccgacgtggcgatgatgggcggcggcgtggtgaggggcttcggctgcaggcccgcttcatcgtccggcggcagaaccggggtccgtccagacgttccgttggtaggtcccaaatcctgtcgccctacacagcggcgggtgcgcgaatagtcaaagttcacacacccagccttcacaggtgtgtggctggcggcctgcttgcgactgtccagcgcctggcgtatctctttataaagggccaagcgcgtttctgtttcctgggtgttggcaggaaacgcggggtaactcaagtcctccaaaaaacccgccacaaataaaaaggggttaacccaatatgccttctgggcatgcctatcccacaagaccgtgtccaatccgggacagtgataacgcaaaaatataccgtctatcaaagcatagtttatagccgagggggtctcgtaacgccaatcaagatcgtcagacgggagcggcacacaaggcacctttaatatatcccccacctctcgagccacccgactccgaataacatattcggttgaagacaagcccccccgcacacacaaaaccccaacggcaataagcccgacccaacccaaaatccccatagcgcctagggtcggcacccacagaaacctacagtccccaagtgtttgcccagtaacacaaccacgacgtcgtgccacacaagccccgtatccccgttcccgcgcttttcgttggtttatatacacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactagtccagtgtggtggaattcgccaccatgtgccatcagcagctggtcatctcatggttctccctggtgtttctggcctcacctctggtcgcaatctgggaactgaaaaaggatgtgtacgtggtggagctggactggtatcccgatgcccctggcgagatggtggtgctgacctgcgacacacccgaggaggatggcatcacctggacactggatcagagctccgaggtgctgggaagcggcaagaccctgacaatccaggtgaaggagttcggcgacgccggccagtacacctgtcacaagggaggagaggtgctgagccactccctgctgctgctgcacaagaaggaggatggcatctggtccacagacatcctgaaggatcagaaggagccaaagaacaagaccttcctgcggtgcgaggccaagaattatagcggccggttcacctgttggtggctgaccacaatctccaccgatctgacattttctgtgaagtctagcaggggatcctctgacccacagggagtgacatgcggagcagccaccctgagcgccgagagggtgcgcggcgataacaaggagtacgagtattccgtggagtgccaggaggactctgcctgtccagcagcagaggagtccctgcctatcgaagtgatggtggatgccgtgcacaagctgaagtacgagaattataccagctccttctttatccgggacatcatcaagcccgatccccctaagaacctgcagctgaagcctctgaagaatagcagacaggtggaggtgtcctgggagtaccctgacacctggagcacaccacactcctatttctctctgaccttttgcgtgcaggtgcagggcaagtccaagcgggagaagaaggacagagtgttcaccgataagacatctgccaccgtgatctgtagaaagaacgcctctatcagcgtgagggcccaggaccgctactattctagctcctggtccgagtgggcctctgtgccttgcagcggcggaggaggaggaggatctatgtgcccagcaaggagcctgctgctggtggccacactggtgctgctggatcacctgtccctggcaaggaatctgccagtggcaacccctgacccaggcatgttcccctgcctgcaccacagccagaacctgctgagggccgtgtccaatatgctgcagaaggcccgccagacactggagttttacccttgtaccagcgaggagatcgaccacgaggacatcacaaaggataagacctccacagtggaggcctgcctgccactggagctgaccaagaacgagtcctgtctgaacagccgggagacaagcttcatcaccaacggctcctgcctggcctctagaaagacaagctttatgatggccctgtgcctgtctagcatctacgaggacctgaagatgtatcaggtggagttcaagaccatgaacgccaagctgctgatggaccccaagaggcagatctttctggatcagaatatgctggccgtgatcgacgagctgatgcaggccctgaacttcaatagcgagacagtgcctcagaagtcctctctggaggagccagatttctacaagaccaagatcaagctgtgcatcctgctgcacgcctttcggatcagagccgtgacaatcgaccgcgtgatgtcctatctgaacgcctctggaggaggaggaagcggaggaggaggatccggcggcggcggctccggctctggcgccaccaacttctccctgctgaagcaggcaggcgacgtggaggagaatccaggacctatgcggatctctaagcctcacctgagaagcatctccatccagtgctacctgtgcctgctgctgaacagccactttctgacagaggccggcatccacgtgttcatcctgggctgttttagcgccggcctgccaaagaccgaggcaaactgggtgaatgtgatctctgacctgaagaagatcgaggatctgatccagagcatgcacatcgatgccacactgtataccgagtctgacgtgcaccctagctgcaaggtgaccgccatgaagtgtttcctgctggagctgcaggtcatcagcctggagtccggcgacgcaagcatccacgatacagtggagaacctgatcatcctggccaacaatagcctgagctccaacggcaatgtgaccgagtccggctgcaaggagtgtgaggagctggaggagaagaacatcaaggagttcctgcagagctttgtgcacatcgtgcagatgttcatcaatacatccggaggaggaggatcaggcggaggaggaagcggcggcggcggctctggcagcggcgccaccaacttttccctgctgaagcaggccggcgatgtggaagaaaatccaggaccaatggcaccaaggagagcaaggggatgcagaaccctgggcctgcctgccctgttattactgctgctgctgaggccaccagcaacaaggggaatcacctgtcctccacccatgagcgtggagcacgccgacatctgggtgaagtcctactctctgtattctagggagcggtacatctgcaacagcggctttaagaggaaggccggcacatctagcctgaccgagtgcgtgctgaacaaggccaccaatgtggcccactggaccacaccttccctgaagtgcatcagggatccagccctggtgcaccagcgccccgcacctccaagcacagtgaccacagcaggagtgacccctcagccagagagcctgtccccttctggcaaggagccagcagcatcctctccaagctccaacaatacagcagcaaccacagcagcaatcgtgccaggctcccagctgatgccaagcaagtccccctctaccggcaccacagagatctctagccacgagtcctctcacggcacaccaagccagaccacagccaagaattgggagctgaccgcaagcgcctcccaccagccacctggcgtgtacccacagggacactccgacaccacagtggccatctctaccagcacagtgctgctgtgcggcctgtctgccgtgagcctgctggcctgttatctgaagtccaggcagaccccaccactggcatctgtggagatggaggccatggaggccctgcccgtcacatgggggactagtagtagagacgaggatctggaaaactgtagtcaccatctgtaagatatccatcacactggcggccgctcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggggatccactagttctagagcggccgccgcacataaaggcccggcgcgaccgacgcccgcagacggcgccggccacgaacgacgggagcggctgcggagcacgcggaccgggagcgggactcgcagagggccgtcggagcggacggcgtcggcatcgcgacgccccggctcgggatcgggatcgcatcggaaagggacacgcggaaagacccacccaccccacccacgaaacacaggggacgcaccccgggggcctccgacgacagaaacccaccggtccgcctttgtgcacgggtaagcaccttgggtgggcggaggaggggggacgcgggggcggaggagggggctcacccgcgttcgtgccttcccgcaggaggaacgtcctcgtcgaggcgaccggcggcgaccgttgcgtggaccgcttcctgctcgtcggggcgaccggcggcgaccgttgcgtggaccgcttcctgctcgtcgggcgggggagcatgtcgtgggccctggaaatggcggacaccttcctggacaacatgcgggttgggcccaggacgtacgccgacgtacgcgatgagatcaataaaagggggcgtgaggaccgggaggcggccaaaaccgccgtgcacgacccggagcgtcctctgctgcgctctcccgggctgctgcccaaaatcgcccccaacgcatccttgggtgtggcacatcgaagaaccggcgggaccgtgaccgacagtccccgtaatccggtaacccgttgagtcccgggtacgaccatcgcccaagtttctgggcggagggtggttccccccgtggctctcgagatgagccagacccaacccccggccccagttgggccgggcgacccagatgtttacttaaaaggcgtgccgtccgccggcatgcaccccagaggtgttcacgcacctcgaggacacccgcgcatgatctccggacccccgcaacggggtgataatgatcaagcggcggggcaatgtggagattcgggtctactacgagtcggtgcggacactacgatctcgaagccatctgaagccgtccgaccgccaacaatccccaggacaccgcgtgttccccgggagccccgggttccgcgaccaccccgagaacctagggaacccagagtaccgcgagctcccagagaccccagggtaccgcgtgaccccagggatccacgacaaccccgggagccccggcctccccgggagccccggaccccacgcaccccccgcgaaccacgtacggctcgcgggtctgtatagcccgggcaagtatgcccccctggcgagcccagaccccttctccccacaagatggagcgtacgctcgggcccgcgtagggctccacaccgcggttcgcgtcccgcccaccggaagcccaacccacacgcacttgcggcatgacccgggcgatgagccaacctcggatgactcagggctctaccctctggacgcccgggcgcttgcgcacctggtgatgttgcccgcggaccaccgggccttctttcgaaccgtggtcgaggtgtctcgcatgtgcgctgcaaacgtgcgcgatcccccgcccccggctacaggggccatgttgggccgccacgcgcggctggtccacacccagtggctccgggccaaccaagagacgtcgcccctgtggccctggcggacggcggccattaactttatcaccaccatggccccccgcgtccaaacccaccgacacatgcacgacctgttgatggcctgtgctttctggtgctgtctgacacacgcatcgacgtgttcgtacgcggggctgtactcgacccactgcctgcatctgtttggtgcgtttgggtgtggggacccggccctaaccccacccctgtgctag

(19) United States
(12) Patent Application Publication
(10) Pub. No.: US 2025/0243252 A1
(43) Pub. Date: Jul. 31, 2025

(54) ONCOLYTIC HERPES SIMPLEX VIRUS VECTORS EXPRESSING IMMUNE SYSTEM-STIMULATORY MOLECULES

(71) Applicant: Virogin Biotech Canada Ltd., Richmond (CA)

(72) Inventors: William Jia, Burnaby (CA); Guoyu Liu, Richmond (CA); Erica Lee, Burnaby (CA); Dmitry Chouljenko, Vancouver (CA); Jun Ding, Vancouver (CA)

(21) Appl. No.: 18/974,616

(22) Filed: Dec. 9, 2024

Related U.S. Application Data
(63) Continuation of application No. 16/322,903, filed on Feb. 1, 2019, now Pat. No. 12,162,919.
(60) Provisional application No. 62/369,646, filed on Aug. 1, 2016.

(51) Int. Cl. C07K 14/54; A61K 35/763; A61K 38/00; A61P 35/00; C07K 14/035; C07K 14/715; C12N 15/00; C12N 15/62; C12N 15/85; C12N 15/869

(52) U.S. Cl. CPC C07K 14/5443; A61K 35/763; A61P 35/00; C07K 14/035; C07K 14/5434; C07K 14/7155; C12N 15/00; C12N 15/625; C12N 15/85; A61K 38/00; C12N 15/8695; C12N 2710/16632; C12N 2710/16643

(57) ABSTRACT

An HSV vector comprising an expression cassette for one or more of IL12, IL15, and hIL15Receptor alpha subunit is provided.

### CROSS-REFERENCE TO RELATED APPLICATION

This patent application is a continuation of U.S. application Ser. No. 16/322,903, filed Feb. 1, 2019, which is a national phase application under 35 U.S.C. § 371 of International Application No. PCT/US2017/044993, filed Aug. 1, 2017, which claims the benefit under 35 U.S.C. § 119(e) of U.S. Provisional Patent Application No. 62/369,646 filed Aug. 1, 2016, which applications are incorporated herein by reference in their entirety for all purposes.

### FIELD OF THE INVENTION

The present invention relates generally to oncolytic herpes simplex virus (oHSV) vectors that express molecules that stimulate the immune system.

### REFERENCE TO SEQUENCE LISTING, TABLE OR COMPUTER PROGRAM

The contents of the electronic sequence listing (VIRO404_sequencelisting.text.xml; Size: 576 KB; and Date of Creation: Apr. 22, 2025) is herein incorporated by reference in its entirety.

### BACKGROUND OF THE INVENTION

Oncolytic viruses (OVs) have been a therapeutic arsenal to specifically destroy cancer cells through oncolysis, which is a killing mechanism characterized by cancer cell lysis through the course of virus lytic replication. In addition to the direct cell killing by the virus. Among the various OVs, herpes simplex virus type 1 (“HSV-1”) based OVs are the farthest advanced, e.g., a herpes virus-based OV (T-Vec) has been approved by the U.S. FDA for the treatment of melanoma. Representative examples of HSV vectors include those described in U.S. Pat. Nos. 7,223,593, 7,537,924, 7,063,835, 7,063,851, 7,118,755, 8,277,818, and 8,680,068.

The present invention overcomes shortcomings of current commercial oncolytic viruses, and further provides additional unexpected benefits.

### SUMMARY

Briefly stated, the disclosure relates to an HSV vector comprising an expression cassette for one or more of IL12, IL15 and/or an ILReceptor 15 alpha subunit. Within one embodiment, the expression cassette expresses all of IL12, IL15 and the IL Receptor 15 alpha subunit. Within preferred embodiments the expression cassette expresses murine or human IL12, murine or human IL15, and murine or human IL15Receptor alpha subunit. Within yet other embodiments the expression cassette expresses either murine or human IL12, hIL15, and murine and h15Receptor alpha subunit.

Within one embodiment, the expression cassette expresses all of IL12, IL15 and the IL Receptor 15 alpha subunit. Within one embodiment, the expression cassette comprises mIL12, hIL15, and hIL15Receptor alpha subunit. Within other embodiments, the expression cassette comprises hIL12, hIL15, and hIL15Receptor alpha subunit. In certain embodiments, the nucleic acid sequence encoding a self-cleaving 2A peptide is located in-frame between coding sequences for mIL12, hIL15, and hIL15Receptor alpha subunit. In other embodiments, the HSV vector encodes a self-cleaving 2A peptide. In other embodiments, the one or more IRES sequences is located between the coding sequences for murine or human IL12, hIL15, and hIL15Receptor alpha subunit. In embodiments, the hIL15 and hIL15Receptor alpha subunit are co-expressed using a IRES sequence, and in certain embodiments, the hIL15 and hIL15Receptor alpha subunit are expressed by a bi-directional promoter, which may be bi-CMV in some embodiments. In yet other embodiments, each of the hIL15 and hIL15Receptor alpha subunit is followed by a nucleic acid sequence encoding Lys5 or Glu5. In certain embodiments, the hIL15Receptor alpha subunit is selected from the group consisting of variant 1, variant 2, variant 3 and variant 4.

The vector may further comprise an expression cassette for one or more PD-L1 blocking peptides, may further comprise sequence encoding a peptide linker or one or more IRES sequences (or both) between multiple PD-L1 blocking peptides. In some embodiments, the HSV vector may further comprise sequence encoding an Fc domain linked to the 3′-end of the PD-L1 blocking peptide. In yet other embodiments, the PD-L1 blocking peptides is inserted in between UL3 and UL4 viral genes.

In certain embodiments, the expression cassette is inserted in the terminal repeat region of HSV genome. In embodiments, the HSV vector further comprises an NFkB and an OCT4/SOX2 enhancing element in ICP4 or ICP27 regulatory regions. The HSV vector may have a deletion of the ICP34.5 genes. In other embodiments, the ICP34.5 gene is regulated by a 3′UTR containing target sequences of miRNAs that are under-expressed in tumor cells.

The expression cassette may comprise at least one bidirectional CMV promoter. It may comprise at least one cellular promoter.

In some embodiments, the expression cassette for mIL12/hIL15/hIL15Receptor alpha subunit is inserted in the terminal repeat region where the original viral sequence is replaced by the cassette.

The HSV vector may be either HSV-1 or HSV-2.

This Brief Summary has been provided to introduce certain concepts in a simplified form that are further described in detail below in the Detailed Description. Except where otherwise expressly stated, this brief Summary is not intended to identify key or essential features of the claimed subject matter, nor is it intended to limit the scope of the claimed subject matter.

The details of one or more embodiments are set forth in the description below. The features illustrated or described in connection with one exemplary embodiment may be combined with the features of other embodiments. Thus, any of the various embodiments described herein can be combined to provide further embodiments. Aspects of the embodiments can be modified, if necessary to employ concepts of the various patents, applications and publications as identified herein to provide yet further embodiments. Other features, objects and advantages will be apparent from the description, the drawings, and the claims.

### BRIEF DESCRIPTION OF THE DRAWINGS

Exemplary features of the present disclosure, its nature and various advantages will be apparent from the accompanying drawings and the following detailed description of various embodiments. Non-limiting and non-exhaustive embodiments are described with reference to the accompanying drawings, wherein like labels or reference numbers refer to like parts throughout the various views unless otherwise specified. The sizes and relative positions of elements in the drawings are not necessarily drawn to scale. For example, the shapes of various elements are selected, enlarged, and positioned to improve drawing legibility. The particular shapes of the elements as drawn have been selected for ease of recognition in the drawings. One or more embodiments are described hereinafter with reference to the accompanying drawings in which:

FIGS. 1A and 1B are schematics of exemplary oHSV vectors.

FIG. 2 shows a schematic of the modified ICP34.5 region (SEQ ID NO: 572) for virus hVG161.

FIG. 3 shows a schematic of a modified UL54 promoter region (SEQ ID NO: 573) for virus hVG161.

FIG. 4 shows a schematic of hVG161.viral genome with an insertion of a PD-L1 blocker (SEQ ID NO: 574).

FIG. 5 shows a schematic of hVG161 modified TR region (SEQ ID NO: 575).

FIGS. 6A, 6B and 6C show ELISA and Western blot data for IL-12 expression following hVG161 infection of cells.

FIGS. 7A, 7B and 7C show ELISA and Western blot data for IL-15 expression following hVG161 infection of cells.

FIGS. 8A, 8B and 8C show ELISA and Western blot data for IgG4 expression following hVG161 infection of cells.

FIGS. 9A, 9B and 9C are (A) a schematic of an exemplary construct in which bi-CMV promoter drives expression of the Sushi domain of IL-15Rα and IL-15, and (B-C) a DNA sequence and schematic (SEQ ID No: 557).

FIGS. 10A, 10B and 10C are (A) a schematic of an exemplary construct in which bi-CMV promoter drives expression of IL-15 and IL-15Rα variant 4, and (B-C) a DNA sequence and schematic (SEQ ID No: 558).

FIGS. 11A, 11B and 11C are (A) a schematic of an exemplary construct in which bi-CMV promoter drives expression of IL-15-K5 and IL-15Rα Sushi domain-E5, and (B-C) a DNA sequence and schematic (SEQ ID No: 559).

FIGS. 12A, 12B, 12C and 12D are (A) a schematic of an exemplary construct in which bi-CMV promoter drives expression of IL-15-K5 and IL-15Rα variant 4-E5, and (B-D) a DNA sequence and schematic (SEQ ID No: 560).

FIGS. 13A, 13B, 13C and 13D are (A) a schematic of an exemplary construct in which the EF1α promoter controls expression of IL-15-IRES-IL-15Rα Sushi domain, and (B-D) a DNA sequence and schematic (SEQ ID No: 561).

FIGS. 14A, 14B, 14C and 14D are (A) a schematic of an exemplary construct in which the EF1α promoter controls expression of IL-15-IRES-IL-15Rα variant 4, and (B-D) a DNA sequence and schematic (SEQ ID No: 562).

FIGS. 15A, 15B, 15C and 15D are (A) a schematic of an exemplary construct in which the EF1α promoter controls expression of IL-15K5-IRES-IL-15Rα Sushi domainE5, and (B-D) a DNA sequence and schematic (SEQ ID No: 563.

FIGS. 16A, 16B, 16C and 16D are (A) a schematic of an exemplary construct in which the EF1α promoter controls expression of IL-15K5-IRES-IL-15Rα variant 4E5, and (B-D) a DNA sequence and schematic (SEQ ID No: 564).

FIGS. 17A, 17B, 17C, 17D and 17E are (A) a schematic of an exemplary construct in which the CMV promoter controls expression of IL-12-p2A-IL-15-p2A-IL-15Rα Sushi domain, and (B-E) a DNA sequence and schematic (SEQ ID No: 565).

FIGS. 18A, 18B, 18C, 18D and 18E are (A) a schematic of an exemplary construct in which the CMV promoter controls expression of IL-12-p2A-IL-15-p2A-IL-15Rα variant 1, and (B-E) a DNA sequence and schematic (SEQ ID No: 566).

FIGS. 19A, 19B, 19C and 19D are (A) a schematic of an exemplary construct in which the CMV promoter controls expression of IL-12-p2A-IL-15K5-p2A-IL-15Rα Sushi domainE5, and (B-D) a DNA sequence and schematic (SEQ ID No: 567).

FIGS. 20A, 20B, 20C and 20D are (A) a schematic of an exemplary construct in which the CMV promoter controls expression of IL-12-p2A-IL-15K5-p2A-IL-15Rα variant 1-E5, and (B-D) a DNA sequence and schematic (SEQ ID No: 568).

FIG. 21 is a chart showing percentage inhibition of PD-L1 binding to PD-1 by blocking peptides.

FIGS. 22A, 22B, 22C and 22D show the effect of PD-L1 inhibiting peptides on cytotoxicity of target cells by anti-CD 3 stimulated human peripheral blood mononuclear cells.

FIGS. 23A and 23B show the effects IL-12 alone, IL-15Rα alone, and IL-12 and 1L-15Rα together on production of IFNγ and TNFα in human peripheral blood mononuclear cells.

FIGS. 24A and 24B show the effects of IL-12 and IL-15Rα on cytotoxicity of U87 and MDA-MB-231 tumor cells by peripheral blood mononuclear cells.

FIGS. 25A, 25B, 25C, 25D and 25E show results of cell infection with VG161-PLBh and VG161-15h.

FIGS. 26A, 26B, 26C and 26D show results of in vitro assays for various constructs. FIG. 15A-15B show results of cell transfection with IL-TF-Fc plasmid carrying IL-12, IL-15, and PD-L1 blocker. FIGS. 15C-15D show results of cell infection with a variety of mutant viruses including hVG161.

FIGS. 27A, 27B, 27C, 27D and 27E show results of cell viability assays for hVG161 and HSV-345 on human tumor cell lines and Vero cell line.

FIGS. 28A, 28B, 28C, 28D, 28E, 28F, 28G, 28H, 28I and 28J show results of in vitro assays for various constructs. FIGS. 28A-28E show results of cell viability assays for mVG161 and HSV-345 on mouse tumor cell lines and Vero cell line; FIGS. 28F-28J show the characterization of transgene expression following mVG161 or VG001 infection of CT26 mouse tumor cells.

FIGS. 29A, 29B, 29C, 29D and 29E show results of in vitro characterization of transgene expression following hVG161 or VG001 infection of various cell lines.

FIGS. 30A, 30B, 30C, 30D, 30E, 30F and 30G show results of assays to evaluate the ability of hVG161 to kill a variety of human cancer cells in vitro.

FIGS. 31A, 31B, 31C, 31D, 31E, 31F and 31G show results of in vivo assays for mVG161 and hVG161 constructs.

FIGS. 32A, 32B and 32C show growth curves for different viruses on three different human cell lines.

FIGS. 33A, 33B, 33C and 33D show growth curves of mVG161 and HSV-345 on mouse tumor cell lines and Vero cell line.

FIGS. 34A, 34B, 34C, 34D and 34E show growth curves of hVG161 and HSV-345 on human tumor cell lines and Vero cell line.

FIGS. 35A, 35B, 35C and 35D show the effects of virus modifications.

FIGS. 36A, 36B, 36C and 36D provide in vitro efficacy data.

FIGS. 37A and 37B provide in vivo data.

FIGS. 38A, 38B and 38C provide data on the effect of VG161m in immune response.

### DETAILED DESCRIPTION OF THE INVENTION

The present disclosure may be understood more readily by reference to the following detailed description of preferred embodiments of the invention and the Examples included herein.

### OVERVIEW OF DISCLOSURE

The present invention may be understood more readily by reference to the following detailed description of preferred embodiments of the invention and the Examples included herein. Briefly stated, the present disclosure provides oncolytic herpes simplex virus type 1 or 2 vectors which express immune stimulator molecules. Representative vectors comprise an expression cassette encoding one or more of IL-12, IL-15 and IL-15Rα. Certain vectors encode murine or human IL-12, murine or human IL-15, and murine or human IL-15Rα. Within certain embodiments the vectors encode murine or human IL-12, hIL15, and hIL15Receptor alpha subunit. Within other embodiments the vectors encode hIL-12, HIL15 and HIL15 Receptor alpha subunit. The three proteins may be expressed on one, two, or three transcripts. When expressed on the same transcript, post-transcriptional processing ensues to result in expression of the individual proteins. In such case, the coding regions are separated by IRES sequences or sequences that encode self-cleaving 2A peptides. The coding regions may be expressed by a bidirectional promoter. The HSV vector optionally expresses one or more PD-L1 peptides that can be secreted.

### A. oHSV Vector

An oncolytic virus is a virus that will lyse cancer cells (oncolysis), preferably in a selective manner. Viruses that selectively replicate in dividing cells over non-dividing cells are often oncolytic. Oncolytic viruses suitable for use herein include Herpes Simplex Viruses 1 and 2.

Herpes Simplex Virus (HSV) 1 and 2 are members of the Herpesviridae family, which infects humans. The HSV genome contains two unique regions, which are designated unique long (U_{L}) and unique short (U_{S}) region. Each of these regions is flanked by a pair of inverted terminal repeat sequences. There are about 75 known open reading frames. The viral genome has been engineered to develop oncolytic viruses for use in e.g. cancer therapy. Tumor-selective replication of HSV may be conferred by mutation of the HSV ICP34.5 (also called γ34.5) gene. HSV contains two copies of ICP34.5. Mutants inactivating one or both copies of the ICP34.5 gene are known to lack neurovirulence, i.e. be avirulent/non-neurovirulent and be oncolytic.

Suitable oncolytic HSV may be derived from either HSV-1 or HSV-2, including any laboratory strain or clinical isolate. In some embodiments, the oHSV may be or may be derived from one of laboratory strains HSV-1 strain 17, HSV-1 strain F, or HSV-2 strain HG52. In other embodiments, it may be of or derived from non-laboratory strain JS-1. Other suitable HSV-1 viruses include HrrR3 (Goldsten and Weller, J. Virol. 62, 196-205, 1988), G2O7 (Mineta et al. Nature Medicine. 1(9):938-943, 1995; Kooby et al. The FASEB Journal, 13(11):1325-1334, 1999); G47Delta (Todo et al. Proceedings of the National Academy of Sciences. 2001; 98(11):6396-6401); HSV 1716 (Mace et al. Head & Neck, 2008; 30(8):1045-1051; Harrow et al. Gene Therapy. 2004; 11(22):1648-1658); HF10 (Nakao et al. Cancer Gene Therapy. 2011; 18(3):167-175); NV1020 (Fong et al. Molecular Therapy, 2009; 17(2):389-394); T-VEC (Andtbacka et al. Journal of Clinical Oncology, 2015: 33(25):2780-8); J100 (Gaston et al. PloS one, 2013; 8(11):e81768); M002 (Parker et al. Proceedings of the National Academy of Sciences, 2000; 97(5):2208-2213); NV1042 (Passer et al. Cancer Gene Therapy. 2013; 20(1):17-24); G2O7-IL2 (Carew et al. Molecular Therapy, 2001; 4(3):250-256); rQNestin34.5 (Kambara et al. Cancer Research, 2005; 65(7):2832-2839); G47Δ-mIL-18 (Fukuhara et al. Cancer Research, 2005; 65(23):10663-10668); and those vectors which are disclosed in PCT applications PCT/US2017/030308 entitled “HSV Vectors with Enhanced Replication in Cancer Cells”, and PCT/US2017/018539 entitled “Compositions and Methods of Using Stat1/3 Inhibitors with Oncolytic Herpes Virus”, all of the above of which are incorporated by reference in their entirety.

The oHSV vector may have modifications, mutations, or deletion of at least one γ34.5 gene. The vector lacks intact γ34.5 genes. In some embodiments, both genes are deleted, mutated or modified. In other embodiments, one is deleted and the other is mutated or modified. Either native γ34.5 gene can be deleted. In one embodiment, the terminal repeat, which comprises γ34.5 gene and ICP4 gene, is deleted. Mutations, such as nucleotide alterations, insertions and deletions render the gene inexpressible or the product inactive. The γ34.5 gene may be modified with miRNA target sequences in its 3′ UTR. The target sequences bind miRNAs that are expressed at lower levels in tumor cells than in their normal counterparts. In some embodiments, the modified or mutated γ34.5 gene(s) are constructed in vitro and inserted into the oHSV vector as replacements for the viral gene(s). When the modified or mutated γ34.5 gene is a replacement of only one γ34.5 gene, the other γ34.5 is deleted. The γ34.5 gene may comprise additional changes, such as having an exogenous promoter.

The oHSV may have additional mutations, which may include disabling mutations e.g., deletions, substitutions, insertions), which may affect the virulence of the virus or its ability to replicate. For example, mutations may be made in any one or more of ICP6, ICPO, ICP4, ICP27, ICP47, ICP 24, ICP56. Preferably, a mutation in one of these genes (optionally in both copies of the gene where appropriate) leads to an inability (or reduction of the ability) of the HSV to express the corresponding functional polypeptide. In some embodiments, the promoter of a viral gene may be substituted with a promoter that is selectively active in target cells or inducible upon delivery of an inducer or inducible upon a cellular event or particular environment. In particular embodiments, a tumor-specific promoter drives expression of viral genes essential for replication of HSV. In certain embodiments the expression of ICP4 or ICP27 or both is controlled by an exogenous promoter, e.g., a tumor-specific promoter. Exemplary tumor-specific promoters include survivin or telomerase; other suitable tumor-specific promoters may be specific to a single tumor type and are known in the art. Other elements may be present. In some cases, an enhancer such as NF-kB/OCT4/SOX2 enhancer is present, for example in the regulatory regions of ICP4 or ICP27 or both. As well, the 5′UTR may be exogenous, such as a 5′UTR from growth factor genes such as FGF.

The oHSV may also have genes and nucleotide sequences that are non-HSV in origin. For example, a sequence that encodes a prodrug, a sequence that encodes a cytokine or other immune stimulating factor, a tumor-specific promoter, an inducible promoter, an enhancer, a sequence homologous to a host cell, among others may be in the oHSV genome. Exemplary sequences encode IL12, IL15, OX40L, PD-L1 blocker or a PD-1 blocker. For sequences that encode a product, they are operatively linked to a promoter sequence and other regulatory sequences (e.g., enhancer, polyadenylation signal sequence) necessary or desirable for expression.

The regulatory region of viral genes may be modified to comprise response elements that affect expression. Exemplary response elements include response elements for NF-kB, Oct-3/4-SOX2, enhancers, silencers, cAMP response elements, CAAT enhancer binding sequences, and insulators. Other response elements may also be included. A viral promoter may be replaced with a different promoter. The choice of the promoter will depend upon a number of factors, such as the proposed use of the HSV vector, treatment of the patient, disease state or condition, and ease of applying an inducer (for an inducible promoter). For treatment of cancer, generally when a promoter is replaced it will be with a cell-specific or tissue-specific or tumor-specific promoter. Tumor-specific, cell-specific and tissue-specific promoters are known in the art. Other gene elements may be modified as well. For example, the 5′ UTR of the viral gene may be replaced with an exogenous UTR.

### B. Immune Stimulatory Molecules

The oHSV vector comprises nucleic acid sequences that encode one or more immune stimulatory molecules (e.g., IL-12, IL-15, and IL-15Rα). The amino acid sequences of exemplary IL-12, IL-15 and IL-15Rα are presented in the Sequence Listing (SEQ ID NOs: 1-6). Any DNA sequence that encodes the amino acid sequence is suitable, although generally codons will be chosen for preferential expression in the subject species slated to receive the oHSV.

### 1. IL-12

Interleukin 12 (IL-12) is mainly produced by dendritic cells, macrophages, and monocytes in response to bacteria (e.g., lipopolysaccharides), pathogens or activated T cells. IL-12 can induce IFN gamma production, cell proliferation, and activate natural killer cells and T cells. It is also critical for differentiation of T cells to Th1 cells. IL-12 can also suppress tumor growth. Murine IL-12 is equally active to both murine and human cells, and either is suitable for use in the oHSV vector.

Biologically active IL-12 is a heterodimeric molecule composed of a 35 kDa (p35) and a 40 kDa (p40) subunit that are covalently linked by a disulfide bridge. Simultaneous expression of the two subunits is necessary to produce the heterodimer. In the oHSV vector, IL-12 expression may be achieved in a variety of ways. The two subunits can be expressed in separate constructs, each with a promoter, or expressed in one construct from a bidirectional promoter, or expressed from one construct with elements such as IRES or self-cleaving peptides between the coding regions. Alternatively, the subunits can be expressed as a single chain. For example, a functional single chain IL-12 fusion protein can be produced by linking the coding regions for p40 and p35 with linkers, usually composed of Ser or Gly or a combination of Ser and Gly, such as Ser₅, (Gly₄Ser)₃ or Gly₆Ser (e.g. Lieschke et al. Nature Biotechnology 15:35, 1997; Lode et al. PNAS 95:2475, 1998; see also WO 2015/095249 for an alternative fusion construct). The sequence and length of the linkers is generally chosen to allow for maximal flexibility of the domains (Chen et al., Adv Drug Deliv Rev. 65: 1357, 2013). A computer program can be used to choose the linker sequence. One such program is called LINKER (Crasto and Feng, Protein Eng Design &Selection 13:309). An exemplary single-chain IL-12 has the amino acid sequence of SEQ ID NO: 1. Amino acid substitutions, insertions and deletions may be made as long as the IL-12 retains function.

### 2. IL-15

IL-15 is a cytokine that regulates natural killer cell and T cell activation and proliferation and may have other biological activities. There are at least two isoforms, which differ in the sequence of the signal peptide and have identical mature protein sequences. The sequence of the isoform with the longer signal peptide (sometimes called LSP-IL15) has GenBank (NCBI) accession no. NP 000576, and the one with the shorter signal peptide (sometimes called SSP-IL15) has Accession No. NP 751915. Either isoform is suitable for use in the oHSV vector. Amino acid insertions, deletions and substitutions, such as are found in polymorphisms, may be present as long as the protein binds IL-15.

In some embodiments, IL-15 and IL-15Rα each have a C-terminal peptide are coiled coils that will selectively dimerize. A number of suitable peptides have been taught (see, e. g., Tripet et al. Protein Engineering 9:1029, 1996; Aronsson et al., Sci Rep 5:14063, 2015). Typically, the amino acid sequence of coiled coils have a heptad repetition of hydrophobic (h) and polar (p) residues in a hpphppp pattern. Two exemplary coiled coils are the K coil (KVSALKE, SEQ ID No. 7) and the E coil (EVSALEK, SEQ ID NO. 8). Generally, from 3-6 tandem copies are used. In some embodiments herein, 5 tandem copies of each are used. K5 (KVSALKEKVSALKEKVSALKEKVSALKEKVSALKE, SEQ ID NO. 9) and E5 (EVSALEKEVSALEKEVSALEKEVSALEKEVSALEK, SEQ ID NO. 10). The K coil and the E coil were designed to be oppositely charged, so IL-15 is fused with one coiled coil and IL-15Rα is fused to an oppositely charged coiled coil. An exemplary Sushi domain fused to E5 is shown in SEQ ID NO: 12, an exemplary IL-15Rα variant 4 fused to E5 is shown in SEQ ID NO: 13, an exemplary IL-15Rα variant 1 fused to E5 is shown in SEQ ID NO: 14, and an exemplary IL-15 fused to K5 is shown in SEQ ID NO: 15.

### 3. IL-15Rα Subunit

Interleukin-15 Receptor alpha subunit (IL-15Rα) is one of three subunits of the complex that binds IL-15. The alpha subunit binds IL-15 with high affinity and can bind to it independently of the other subunits. There are at least four variants (isoforms), herein called variant 1 (NP 002180.1) (SEQ ID NO: 3); variant 2 (NP 751950.2) (SEQ ID NO: 4); variant 3 (NP 001230468.1) (SEQ ID NO: 5); and variant 4 (NP_001243694) (SEQ ID NO: 6). The alpha subunit contains a Sushi domain (aka complement control protein (CCP), short consensus repeats (SCRs) or SUSHI repeats) that is the shortest region retaining IL-15 binding activity. A typical Sushi domain is about 60-70 aa containing four cysteines forming two disulfide bonds and is a common motif in protein-protein interactions. The Sushi domain of IL-15Rα encompasses residue 31 to about 95 (in reference to variant 1) (SEQ ID NO: 11). The location of the Sushi domain in the other variants is known. Amino acid substitution of any of the cysteines in sIL-15Rα abolishes its ability to inhibit acute inflammation and T cell response to allogenic antigens in vivo (Wei et al. J Immunol. 167:277, 2001).

The oHSV vector comprises nucleic acid sequence encoding IL-15Rα, a variant of IL-15Rα, or a Sushi domain. Generally, the protein is expressed with a leader peptide, and in some embodiments, the leader peptide is from IL-15Rα. Other leader peptides are known in the art. Amino acid substitutions may be present as long as the protein binds IL-15. Natural substitutions, polymorphisms, are known.

### 4. PD-L1 Blocking Peptide

Programmed death-ligand 1 (PD-L1) plays a role in suppressing the immune system, probably as an effect of binding the PD-1 receptor. Blocking the protein-protein interaction has been shown to improve cancer therapy.

The oHSV vector may express PD-L1 blocking peptides. Suitable peptides include TAHPSPSPRSAGQF (SEQ ID NO: 16), EYRMSPSNQT (SEQ ID NO: 17), YYRMSPSNQT (SEQ ID NO: 18), TRYPSPSPKPEGRF (SEQ ID NO: 19), and WNRLSPSNQT (SEQ ID NO: 20). Other suitable peptides include those in Table 4 (SEQ ID NOs: 21-500). Generally, the blocking peptides are expressed with a leader sequence. Leader sequences are well known in the art. They include the immunoglobulin kappa chain leader sequence (METDTLLLWVLLLWVPGSTG; SEQ ID NO: 501) and the IL-2 leader sequence (MYRMQLLSCIALSLALVTNS; SEQ ID NO: 502). When more than one blocking peptide is present, typically the peptides will be separated by a linker peptide that confers flexibility. The linkers are usually Gly or Ser or Gly/Ser rich. Examples of suitable linkers are shown in (SEQ ID NOs: 503-519) (see also, Chichili et al. Protein Science 22: 153, 2013). There may be one copy of a peptide or two copies or three copies or more. Multiple copies are usually in tandem and may have a linker between the copies. The blocking peptide constructs may also comprise an Fc sequence at the C-terminus of the peptide, or an immunoglobulin Fc sequence with or without the hinge region. While any of the Fc regions are suitable, in general, the Fc will be from one of the IgG subclasses, e.g., human IgG1, human IgG2, human IgG3, and human IgG4 or their murine counterparts.

### C. Organization of Elements

The molecules IL-12, IL-15 and IL-15Rα can be in a variety of different configurations in the oHSV vector. For example, each of the molecules may be individually expressed from a separate promoter/regulatory region or co-expressed from one or two separate promoters/regulatory regions.

In certain embodiments, two or three of the molecules are expressed in a single transcript from one promoter and their coding sequences are separated by IRES (internal ribosome entry site) sequences. IRES regions attract a eukaryotic ribosomal translation initiation complex and thus allow translation initiation in the middle of an mRNA and independently of the commonly utilized 5′-terminal cap structure. Suitable IRES sequences are well known and many may be found in IRESite's database of experimentally verified IRES sequences (see, e.g., iresite.org/IRESite_web.php?page=browse_plasmids; accessed 26 May 2016).

In various embodiments, the three genes are present in any order and separated by one or more IRES sequences. The IRES sequences may be identical or not. Additional sequences may be present at the gene/IRES junction or an IRES/IRES junction.

In certain embodiments, two or three of the molecules are expressed in a single transcript from one promoter and their coding sequences are separated by one or more self-cleaving 2A peptides. These peptides are short (about 18-22 amino acids) and are inserted in-frame between coding sequences. During translation, ribosomes skip the synthesis of the glycyl-prolyl peptide bond at the C-terminus of a 2A peptide, leading to the cleavage between a 2A peptide and its immediate downstream protein. As a result, they produce equimolar levels of multiple gene products from the same mRNA. The “cleavage” occurs between the gly-pro residues at the C-terminus, meaning the upstream cistron will have additional residues added to its C-terminus, while the downstream cistron will begin with a proline. Exemplary p2A peptide sequences are shown in SEQ ID NOs: 520-535.

Another means to effect co-expression of the molecules is to use a bidirectional promoter. Bidirectional promoters are a common feature of the human genome (Trinklein et al. Genome Res 14:62, 2004). A bidirectional promoter initiates transcription in both directions and typically contains shared elements that regulate both genes. In addition to natural bidirectional promoters, promoters have been synthesized to be bidirectional. One such promoter is bi-CMV. pBI-CMV1 is a mammalian bidirectional expression vector that allows the constitutive expression of two proteins of interest. Protein expression is driven by one of two constitutively active, minimal human cytomegalovirus promoters, PminCMV1 and PminCMV2 in opposite orientations. An exemplary DNA sequence of a bidirectional CMV promoter is SEQ ID NO. 536.

The bidirectional promoter (e.g., bi-CMV promoter) is mainly used to achieve co-expression of hIL15 and IL-15Rα (or the Sushi domain). When two molecules are co-expressed using IRES or p2A sequences, generally it will be hIL15 and IL-15Rα (or the Sushi domain). In these cases, IL-12 and PD-L1 blocking peptides may be co-expressed using a bidirectional promoter or as a multicistronic transcript with IRES or p2A sequences, or they may be individually expressed from their own promoters/regulatory regions.

Other promoters may be used. Cellular promoters, viral promoters and the like are suitable. The promoters may be constitutive or inducible or cell/tissue-specific. Many promoters are well known. One particular promoter that may be useful is the constitutive EF-1α promoter.

The sequences are assembled in one or more expression cassettes. The Examples provide exemplary versions of some expression cassettes. The expression cassette may be inserted into the HSV genome in any location that does not disrupt critical functions (e.g., replication). In certain embodiments, the cassette is inserted in internal or in terminal repeat region after first deleting the repeat. Other suitable areas for insertion include between viral genes, such as, for example, the UL3 and UL4 viral genes, the UL50 and UL51 genes, and between US1 and US2.

In certain embodiments, a cassette expressing PD-L1 blocking peptides in inserted in between viral genes (e.g., UL3 and UL4, UL50 and UL51 and/or US1 and US2). In other embodiments, a cassette expressing IL-12, IL-15 and IL-15Rα is inserted in place of the terminal repeat region and a cassette expressing PD-L1 peptides is inserted in between UL3 and UL4 genes.

### D. Therapeutic Compositions

Therapeutic compositions are provided that may be used to prevent, treat, or ameliorate the effects of a disease, such as, for example, cancer. More particularly, therapeutic compositions are provided comprising at least one oncolytic virus as described herein. Representative examples include an oHSV that has an expression cassette for one or more of IL12, IL15 and/or ILReceptor 15 alpha subunit. Within one embodiment, the expression cassette expresses all of IL12, IL15 and the IL Receptor 15 alpha subunit. Within preferred embodiments, the expression cassette comprises murine or human IL12, hIL15, and hIL15Receptor alpha subunit.

In certain embodiments, the compositions will further comprise a pharmaceutically acceptable carrier. The phrase “pharmaceutically acceptable carrier” is meant to encompass any carrier, diluent or excipient that does not interfere with the effectiveness of the biological activity of the oncolytic virus and that is not toxic to the subject to whom it is administered (see generally Remington: The Science and Practice of Pharmacy, Lippincott Williams & Wilkins; 21st ed. (May 1, 2005 and in The United States PharmacopE1A: The National Formulary (USP 40—NF 35 and Supplements).

In the case of an oncolytic virus as described herein, non-limiting examples of suitable pharmaceutical carriers include phosphate buffered saline solutions, water, emulsions (such as oil/water emulsions), various types of wetting agents, sterile solutions, and others. Additional pharmaceutically acceptable carriers include gels, bioadsorbable matrix materials, implantation elements containing the oncolytic virus, or any other suitable vehicle, delivery or dispensing means or material(s). Such carriers can be formulated by conventional methods and can be administered to the subject at an effective dose. Additional pharmaceutically acceptable excipients include, but are not limited to, water, saline, polyethyleneglycol, hyaluronic acid and ethanol. Pharmaceutically acceptable salts can also be included therein, e.g., mineral acid salts (such as hydrochlorides, hydrobromides, phosphates, sulfates, and the like) and the salts of organic acids (such as acetates, propionates, malonates, benzoates, and the like). Such pharmaceutically acceptable (pharmaceutical-grade) carriers, diluents and excipients that may be used to deliver the oHSV to a target cancer cell will preferably not induce an immune response in the individual (subject) receiving the composition (and will preferably be administered without undue toxicity).

The compositions provided herein can be provided at a variety of concentrations. For example, dosages of oncolytic virus can be provided which ranges from about 10⁶ to about 10⁹ pfu. Within further embodiments, the dosage form can range from about 10⁶ to about 10⁸ pfu/ml, with up to 4 mls being injected into a patient with large lesions (e.g., >5 cm) and smaller amounts (e.g., up to 0.1 mls) in patients with small lesions (e.g., <0.5 cm) every 2-3 weeks, of treatment.

Within certain embodiments of the invention, lower dosages than standard may be utilized. Hence, within certain embodiments less than about 10⁶ pfu/ml (with up to 4 mls being injected into a patient every 2-3 weeks) can be administered to a patient.

The compositions may be stored at a temperature conducive to stable shelf-life, and includes room temperature (about 20° C.), 4° C., −20° C., −80° C., and in liquid N2. Because compositions intended for use in vivo generally don't have preservatives, storage will generally be at colder temperatures. Compositions may be stored dry (e.g., lyophilized) or in liquid form.

### E. Administration

In addition to the compositions described herein, various methods of using such compositions to treat or ameliorate cancer are provided, comprising the step of administering an effective dose or amount of a HSV vector as described herein to a subject.

The terms “effective dose” and “effective amount” refers to amounts of the oncolytic virus that is sufficient to effect treatment of a targeted cancer, e.g., amounts that are effective to reduce a targeted tumor size or load, or otherwise hinder the growth rate of targeted tumor cells. More particularly, such terms refer to amounts of oncolytic virus that is effective, at the necessary dosages and periods of treatment, to achieve a desired result. For example, in the context of treating a cancer, an effective amount of the compositions described herein is an amount that induces remission, reduces tumor burden, and/or prevents tumor spread or growth of the cancer. Effective amounts may vary according to factors such as the subject's disease state, age, gender, and weight, as well as the pharmaceutical formulation, the route of administration, and the like, but can nevertheless be routinely determined by one skilled in the art.

The therapeutic compositions are administered to a subject diagnosed with cancer or is suspected of having a cancer. Subjects may be human or non-human animals.

The compositions are used to treat cancer. The terms “treat” or “treating” or “treatment,” as used herein, means an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, diminishment of the reoccurrence of disease, and remission (whether partial or total), whether detectable or undetectable. The terms “treating” and “treatment” can also mean prolonging survival as compared to expected survival if not receiving treatment.

Representative forms of cancer include carcinomas, leukemia's, lymphomas, myelomas and sarcomas. Further examples include, but are not limited to cancer of the bile duct cancer, brain (e.g., glioblastoma), breast, cervix, colorectal, CNS (e.g., acoustic neuroma, astrocytoma, craniopharyogioma, ependymoma, glioblastoma, hemangioblastoma, medulloblastoma, menangioma, neuroblastoma, oligodendroglioma, pinealoma and retinoblastoma), endometrial lining, hematopoietic cells (e.g., leukemia's and lymphomas), kidney, larynx, lung, liver, oral cavity, ovaries, pancreas, prostate, skin (e.g., melanoma and squamous cell carcinoma) and thyroid. Cancers can comprise solid tumors (e.g., sarcomas such as fibrosarcoma, myxosarcoma, liposarcoma, chondrosarcoma and osteogenic sarcoma), be diffuse (e.g., leukemia's), or some combination of these (e.g., a metastatic cancer having both solid tumors and disseminated or diffuse cancer cells). Cancers can also be resistant to conventional treatment (e.g. conventional chemotherapy and/or radiation therapy).

Benign tumors and other conditions of unwanted cell proliferation may also be treated.

The oHSV as described herein may be given by a route that is e.g. oral, topical, parenteral, systemic, intravenous, intramuscular, intraocular, intrathecal, intratumor, subcutaneous, or transdermal. Within certain embodiments the oncolytic virus may be delivered by a cannula, by a catheter, or by direct injection. The site of administration may be intra-tumor or at a site distant from the tumor. The route of administration will often depend on the type of cancer being targeted.

The optimal or appropriate dosage regimen of the oncolytic virus is readily determinable within the skill of the art, by the attending physician based on patient data, patient observations, and various clinical factors, including for example a subject's size, body surface area, age, gender, and the particular oncolytic virus being administered, the time and route of administration, the type of cancer being treated, the general health of the patient, and other drug therapies to which the patient is being subjected. According to certain embodiments, treatment of a subject using the oncolytic virus described herein may be combined with additional types of therapy, such as chemotherapy using, e.g., a chemotherapeutic agent such as etoposide, ifosfamide, adriamycin, vincristin, doxicyclin, and others.

oHSV may be formulated as medicaments and pharmaceutical compositions for clinical use and may be combined with a pharmaceutically acceptable carrier, diluent, excipient or adjuvant. The formulation will depend, at least in part, on the route of administration. Suitable formulations may comprise the virus and inhibitor in a sterile medium. The formulations can be fluid, gel, paste or solid forms. Formulations may be provided to a subject or medical professional

A therapeutically effective amount is preferably administered. This is an amount that is sufficient to show benefit to the subject. The actual amount administered and time-course of administration will depend at least in part on the nature of the cancer, the condition of the subject, site of delivery, and other factors.

Within yet other embodiments of the invention the oncolytic virus can be administered intratumorally, or, after surgical resection of a tumor.

The following examples are offered by way of illustration, and not by way of limitation.

### EXAMPLES

All constructs are generated using standard recombinant techniques, including chemical synthesis.

### Example 1

### Schematic of Exemplary oHSV Vectors

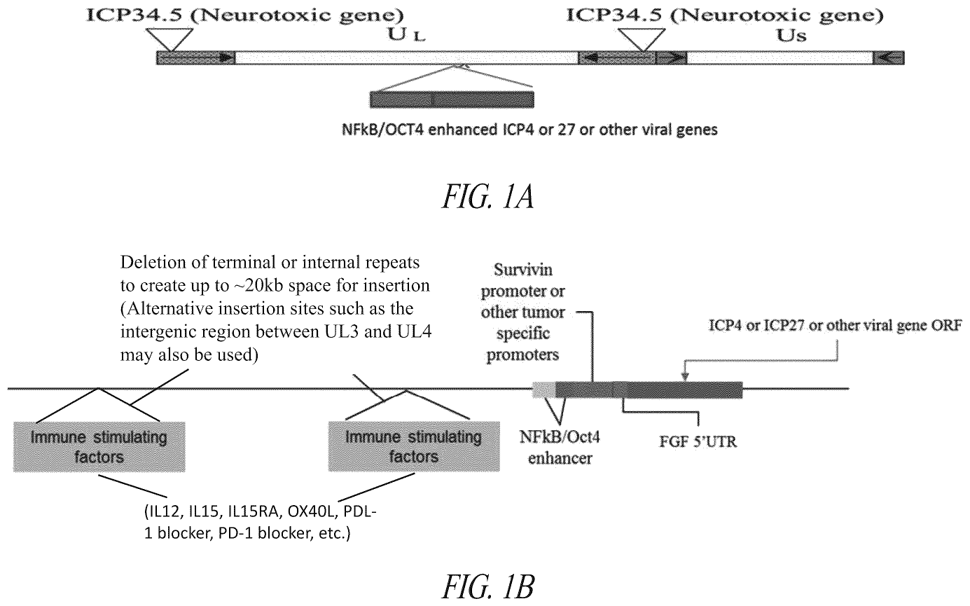

FIGS. 1A and 1B provide and exemplary schematic of representative oHSV vectors.

### Example 2

### Exemplary Constructs

In this example, various constructs and their sequences are presented.

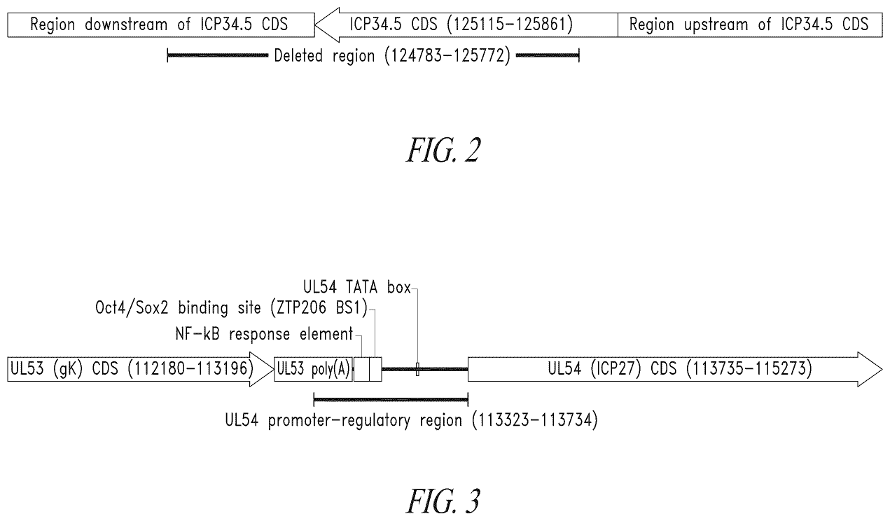

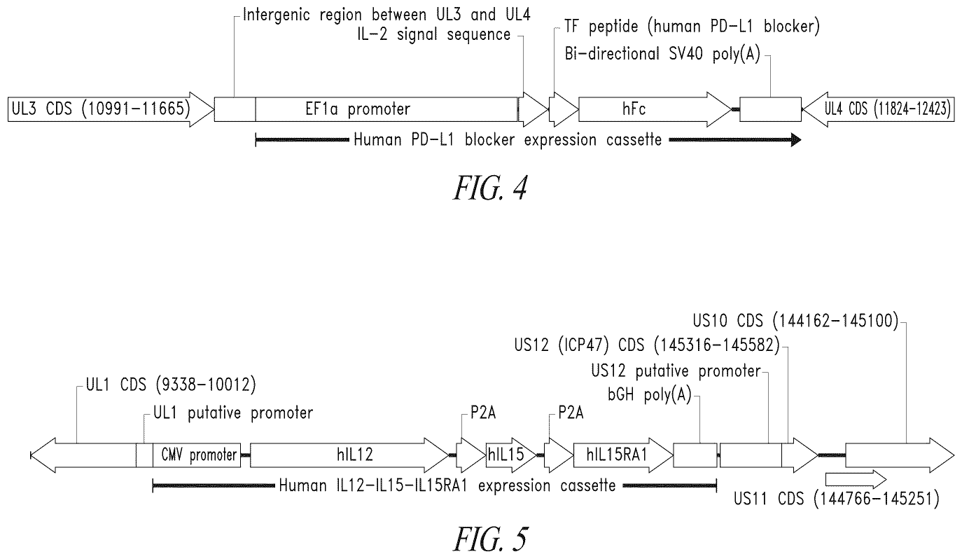

hVG161 comprises a modified ICP34.5 region (FIG. 2; SEQ ID NO.572), a modified UL54 promoter-regulatory region (FIG. 3; SEQ ID NO. 573), an insertion of a PD-L1 blocker within the intergenic region between UL3 and UL4 (FIG. 4; SEQ ID No. 574), and a modified terminal repeat (TR) region carrying an expression cassette encoding IL-12, IL-15, and IL-15 receptor alpha subunit (FIG. 5; SEQ ID NO. 575). These four viruses also have a modified and partially deleted ICP 34.5 region.

mVG161 is a functionally identical mouse version of hVG161 except that that mVG161 carries a mouse version of IL-12 and a mouse PD-L1 blocker in the same location on the viral genome where hVG161 carries a human IL-12 and a human PD-L1 blocker.

### Example 3

### Abbreviations Used in Subsequent Examples

TF-Fc: PD-L1 blocking peptide (TF) fused to Fc and used for construction of VG161.
IL-TF-Fc: plasmid carrying IL-12, IL-15, and PD-L1 blocker.
HSV-345: ICP34.5-deleted virus.
OS-ICP27 2-11: ICP34.5-deleted virus with Oct4/Sox2 binding site and surviving promoter (OS) inserted within the promoter-regulatory region of ICP27 (OS-ICP27) that was not used for construction of VG161.
OS-ICP27 5-7: ICP34.5-deleted virus with OS-ICP27 mutation that was not used for construction of VG161.
NO-ICP27 1-4-4 (also known as NO-ICP27-145): ICP34.5-deleted virus with NF-kB response element and Oct4/Sox2 binding site (NO) inserted within the promoter-regulatory region of ICP27 (NO-ICP27) at a location 145 bp upstream of the transcription start site of ICP27 and that was used for construction of VG161.
NO-ICP27 5-2-2 (also known as NO-ICP27-99): ICP34.5-deleted virus with NF-kB response element and Oct4/Sox2 binding site (NO) inserted within the promoter-regulatory region of ICP27 (NO-ICP27) at a location 99 bp upstream of the transcription start site of ICP27 and that was not used for construction of VG161.
VG001 (also known as VG160): backbone virus that was used for construction of VG161 (NO-ICP27 1-4-4 mutant carrying an exogenous promoter and poly (A) flanking an empty MCS within deleted terminal repeat region of the viral genome that is subsequently used for insertion of the IL-12/IL-15 expression cassette).
VG001-15h (also known as VG161-15h): VG001 carrying human IL-15.
VG001-1215h (also known as VG161-1215h): VG001 carrying human IL-12 and human IL-15.
VG001-PLBh (also known as VG161-PLBh): VG001 carrying human PD-L1 blocker inserted within intergenic region between UL3 and UL4.
8-8-15RA1-PDL1b: VG001 carrying human IL-15 and human PD-L1 blocker.
VG161-1215PLBm (also known as mVG161): VG001 carrying mouse IL-12, human IL-15, and mouse PD-L1 blocker.
VG161-1215PLBh (also known as hVG161 or VG161): VG001 carrying human IL-12, human IL-15, and human PD-L1 blocker.

### Example 4

### Expression of IL-12 Following Infection of Cells by hVG161

In this Example, Western blot and ELISA data of Il-12 expression is shown.

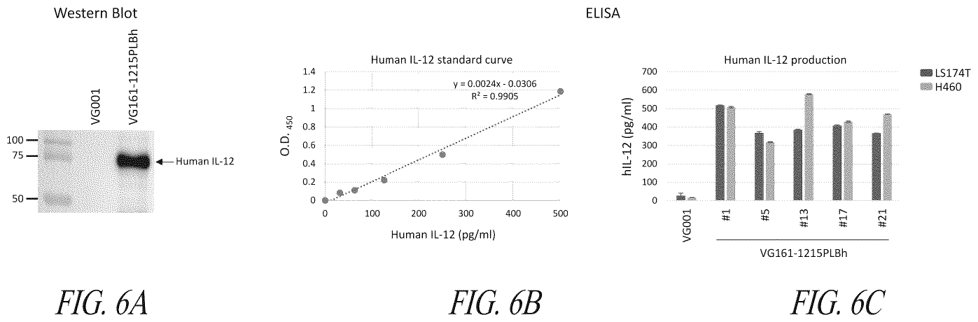

FIG. 6A shows Western Blot results after VG161-1215PLBh virus infection. H460 tumour cells were infected with VG161-1215PLB or VG001 virus (MOI=1) for 24 hours. Cell lysates were prepared, ran on 12% SDS-PAGE gel, and transferred to PVDF membrane. The membrane was blotted with anti-human IL-12 antibody followed by HRP-conjugated anti-mouse IgG secondary antibody and the mage was detected and analyzed using Bio-Rad ImageLab system.

FIGS. 6B-6C shows that production of human IL-12 is upregulated after VG161-1215PLBh virus infection. LS174T or H460 tumour cells were infected with VG161-1215PLB or VG001 virus (MOI=1) for 48 hours. Infected cell supernatants were harvested and bound to anti-human IL-12 capture antibody coated 96-well Immuno Maxisorp flat bottom plate. Binding was detected via a biotinylated anti-human IL-12 antibody, avidin-horseradish peroxidase (HRP), and 3,3′,5,5′-Tetramethylbenzidine (TMB) substrate. Absorbance measurements were collected at 450 nm via a plate reader. The concentration of human IL-12 in cultured supernatants was calculated based on human IL-12 standard curve.

### Example 5

### Expression of IL-15 Following Infection of Cells by hVG161

In this Example, Western blot and ELISA data of IL-15 expression is shown.

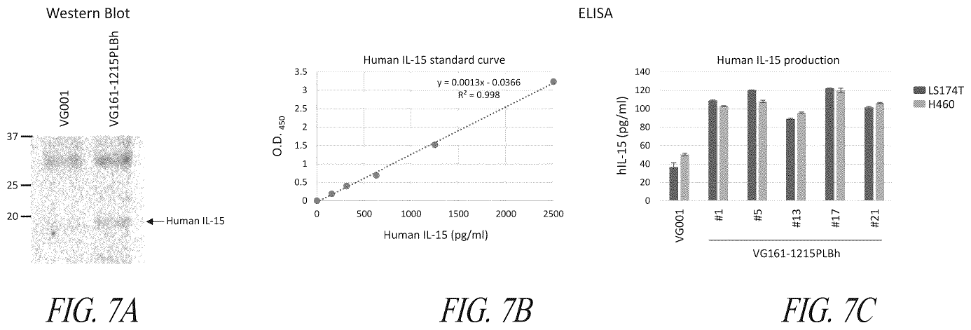

FIG. 7A shows Western Blot results after VG161-1215PLBh virus infection. H460 tumour cells were infected with VG161-1215PLB or VG001 virus (MOI=1) for 24 hours. Cell lysates were prepared, ran on 12% SDS-PAGE gel, and transferred to PVDF membrane. The membrane was blotted with anti-human IL-15 antibody followed by HRP-conjugated anti-mouse IgG secondary antibody and the image was detected and analyzed using Bio-Rad ImageLab system.

FIGS. 7B-7C shows that production of human IL-15 is upregulated after VG161-1215PLBh virus infection. LS174T or H460 tumour cells were infected with VG161-1215PLB or VG001 virus (MOI=1) for 48 hours. Infected cell supernatants were harvested and bound to anti-human IL-15 capture antibody coated 96-well Immuno Maxisorp flat bottom plate. Binding was detected via a biotinylated anti-human IL-15 antibody, avidin-horseradish peroxidase (HRP), and 3,3′,5,5′-Tetramethylbenzidine (TMB) substrate. Absorbance measurements were collected at 450 nm via a plate reader. The concentration of human IL-15 in cultured supernatants was calculated based on human IL-15 standard curve.

### Example 6

### Expression of IgG4 Following Infection of Cells by hVG161

In this Example, Western blot and ELISA data of IgG4 expression is shown.

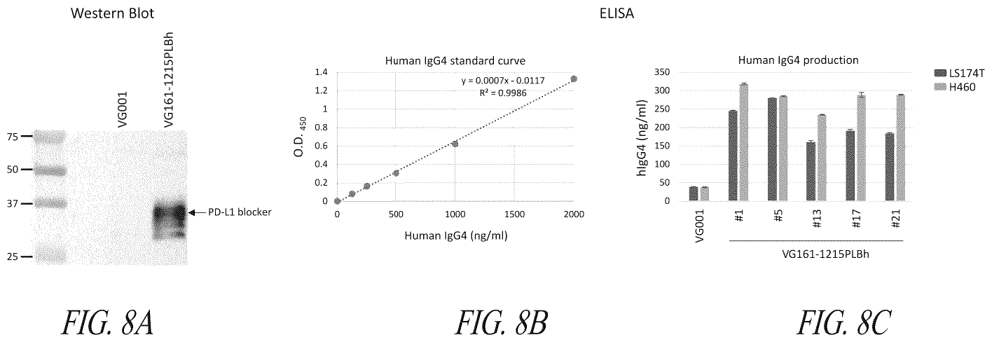

FIG. 8A shows Western Blot results after VG161-1215PLBh virus infection. H460 tumour cells were infected with VG161-1215PLB or VG001 virus (MOI=1) for 24 hours. Cell lysates were prepared, ran on 12% SDS-PAGE gel, and transferred to PVDF membrane. The membrane was blotted with HRP-conjugated anti-human IgG antibody and the image was detected and analyzed using Bio-Rad ImageLab system.

FIGS. 8B-8C shows that production of human PD-L1 blocker (fused to human Fc domain) is upregulated after VG161-1215PLBh virus infection. LS174T or H460 tumour cells were infected with VG161-1215PLB or VG001 virus (MOI=1) for 48 hours. Infected cell supernatants were harvested and bound to anti-human IgG4 capture antibody coated 96-well Immuno Maxisorp flat bottom plate. Binding was detected via a biotinylated anti-human IgG4 antibody, avidin-horseradish peroxidase (HRP), and 3,3′,5,5′-Tetramethylbenzidine (TMB) substrate. Absorbance measurements were collected at 450 nm via a plate reader. The concentration of human IgG4 in cultured supernatants was calculated based on human IgG4 standard curve.

### Example 7

### Constructs Comprising PD-L1 Blocking Peptides

PD-L1 blocking peptides are generated with an Ig κ chain leader sequence (SEQ ID NO: 501). When two or more blocking peptides are in the same construct, they are linked with a Gly-Ser rich sequence (Gly₄Ser)₃ (SEQ ID NO: 503). The following constructs are made.

TF alone:
(SEQ ID NO: 537)
METDTLLLWVLLLWVPGSTGTAHPSPSPRSAGQF;
ET + TF:
(SEQ ID NO: 538)
METDTLLLWVLLLWVPGSTGEYRMSPSNQTGGGGSGGGGSGGGGSTAHP
SPSPRSAGQF;
YT + TF:
(SEQ ID NO: 539)
METDTLLLWVLLLWVPGSTGYYRMSPSNQTGGGGSGGGGSGGGGSTAHP
SPSPRSAGQF;
Mouse TF:
(SEQ ID NO: 540)
METDTLLLWVLLLWVPGSTGTRYPSPSPKPEGRF;
Mouse WT + TF:
(SEQ ID NO: 541)
METDTLLLWVLLLWVPGSTGWNRLSPSNQTGGGGSGGGGSGGGGSTRYP
SPSPKPEGRF.
Triple TF + ET:
(SEQ ID NO: 542)
METDTLLLWVLLLWVPGSTGTAHPSPSPRSAGQFTAHPSPSPRSAGQFT
AHPSPSPRSAGQFGGGGSGGGGSGGGGSEYRMSPSNQTEYRMSPSNQTE
YRMSPSNQT
(SEQ ID NO: 543)
METDTLLLWVLLLWVPGSTGEYRMSPSNQTEYRMSPSNQTEYRMSPSNQ
TGGGGSGGGGSGGGGSTAHPSPSPRSAGQFTAHPSPSPRSAGQFTAHPS
PSPRSAGQF.

Other constructs are made using an IL-2 signal sequence (MYRMQLLSCIALSLALVTNS (SEQ ID NO: 502), and the human IgG4 Fc region (with hinge region) (SEQ ID NO: 544) or the murine IgG1 Fc region (with hinge region) (SEQ ID NO: 545). The constructs are:

TF alone:
(SEQ ID NO: 546)
MYRMQLLSCIALSLALVTNSTAHPSPSPRSAGQFISAMVRSPPCPSCPA
PEFLGGPSVFLFPPKPKDTLMISRTPEVTCVVVDVSQEDPEVQFNWYVD
GVEVHNAKTKPREEQFNSTYRVVSVLTVLHQDWLNGKEYKCKVSNKGLP
SSIEKTISKAKGQPREPQVYTLPPSQEEMTKNQVSLTCLVKGFYPSDIA
VEWESNGQPENNYKTTPPVLDSDGSFFLYSRLTVDKSRWQEGNVFSCSV
MHEALHNHYTQKSLSLSPGK
ET + TF:
(SEQ ID NO: 547)
MYRMQLLSCIALSLALVTNSEYRMSPSNQTGGGGSGGGGSGGGGSTAHP
SPSPRSAGQFISAMVRSPPCPSCPAPEFLGGPSVFLFPPKPKDTLMISR
TPEVTCVVVDVSQEDPEVQFNWYVDGVEVHNAKTKPREEQFNSTYRVVS
VLTVLHQDWLNGKEYKCKVSNKGLPSSIEKTISKAKGQPREPQVYTLPP
SQEEMTKNQVSLTCLVKGFYPSDIAVEWESNGQPENNYKTTPPVLDSDG
SFFLYSRLTVDKSRWQEGNVFSCSVMHEALHNHYTQKSLSLSPGK
YT + TF:
(SEQ ID NO: 548)
MYRMQLLSCIALSLALVTNSYYRMSPSNQTGGGGSGGGGSGGGGSTAHP
SPSPRSAGQFISAMVRSPPCPSCPAPEFLGGPSVFLFPPKPKDTLMISR
TPEVTCVVVDVSQEDPEVQFNWYVDGVEVHNAKTKPREEQFNSTYRVVS
VLTVLHQDWLNGKEYKCKVSNKGLPSSIEKTISKAKGQPREPQVYTLPP
SQEEMTKNQVSLTCLVKGFYPSDIAVEWESNGQPENNYKTTPPVLDSDG
SFFLYSRLTVDKSRWQEGNVFSCSVMHEALHNHYTQKSLSLSPGK
Mouse TF:
(SEQ ID NO: 549)
MYRMQLLSCIALSLALVTNSTRYPSPSPKPEGRFISAMVRSGCKPCICT
VPEVSSVFIFPPKPKDVLTITLTPKVTCVVVDISKDDPEVQFSWFVDDV
EVHTAQTQPREEQFNSTFRSVSELPIMHQDWLNGKEFKCRVNSAAFPAP
IEKTISKTKGRPKAPQVYTIPPPKEQMAKDKVSLTCMITDFFPEDITVE
WQWNGQPAENYKNTQPIMDTDGSYFVYSKLNVQKSNWEAGNTFTCSVLH
EGLHNHHTEKSLSHSPGK
Mouse WT + TF:
(SEQ ID NO: 550)
MYRMQLLSCIALSLALVTNSWNRLSPSNQTGGGGSGGGGSGGGGSTRYP
SPSPKPEGRFISAMVRSGCKPCICTVPEVSSVFIFPPKPKDVLTITLTP
KVTCVVVDISKDDPEVQFSWFVDDVEVHTAQTQPREEQFNSTFRSVSEL
PIMHQDWLNGKEFKCRVNSAAFPAPIEKTISKTKGRPKAPQVYTIPPPK
EQMAKDKVSLTCMITDFFPEDITVEWQWNGQPAENYKNTQPIMDTDGSY
FVYSKLNVQKSNWEAGNTFTCSVLHEGLHNHHTEKSLSHSPGK.

### Example 8

### Constructs Comprising IL-15 and IL-15Rα Under Control of a Bidirectional CMV Promoter

In this example, a variety of constructs are generated to co-express IL-15 and IL-15Rα under control of a bidirectional CMV promoter.

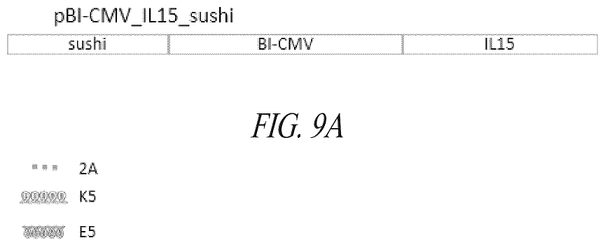

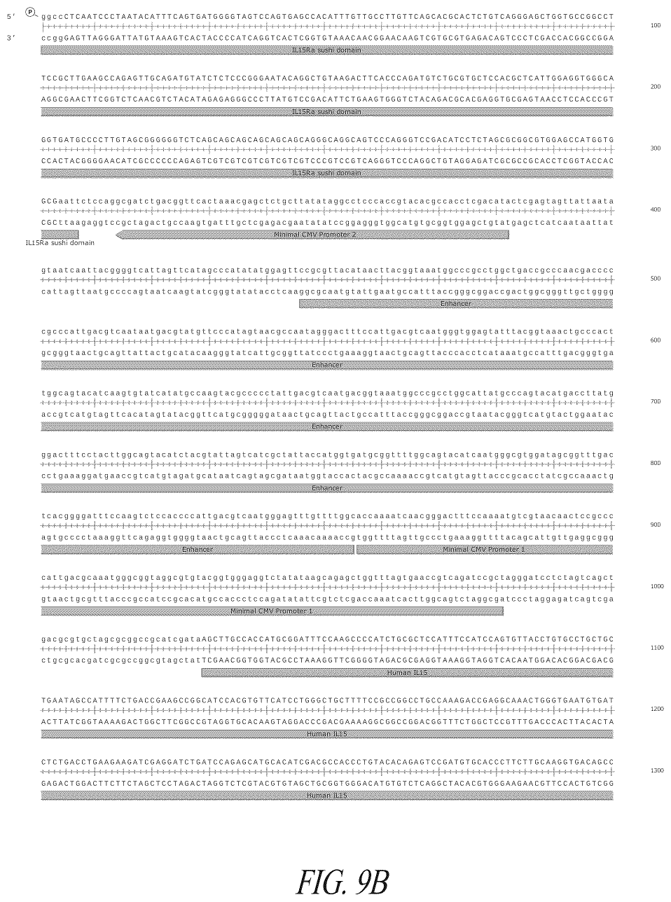

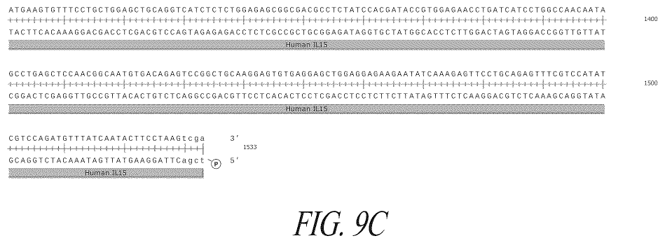

In construct 1, bi-CMV promoter drives expression of the Sushi domain of IL-15Rα and IL-15 (FIG. 9, SEQ ID No. 557).

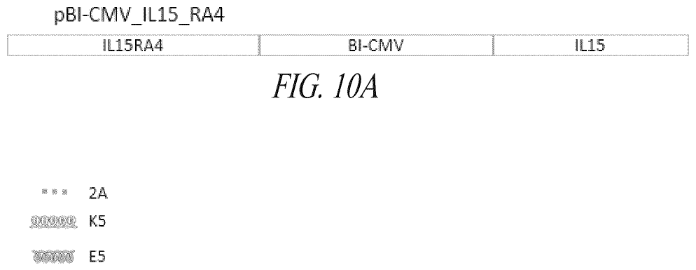

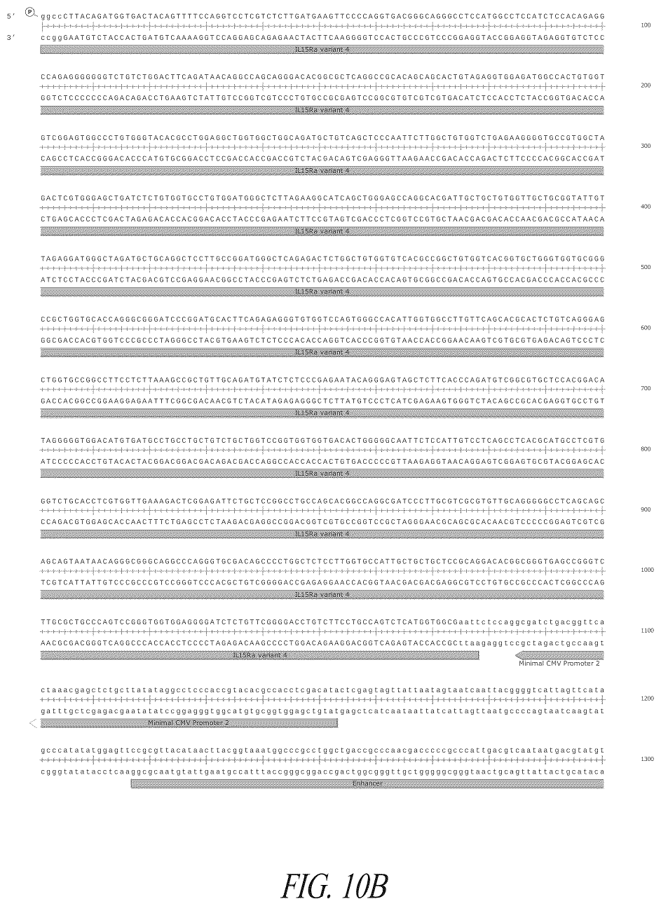

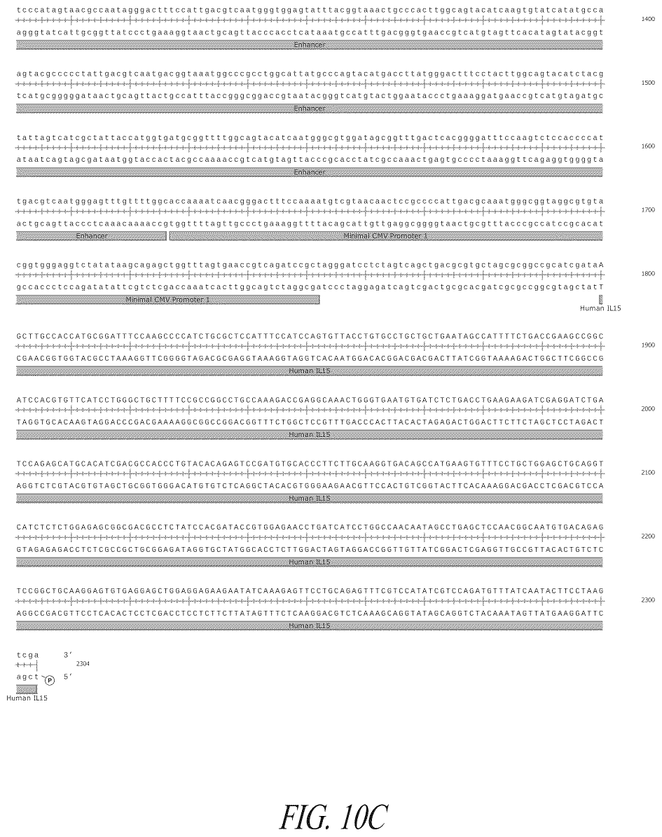

In construct 2, bi-CMV promoter drives expression of IL-15 and IL-15Rα variant 4 (FIG. 10, SEQ ID No. 558).

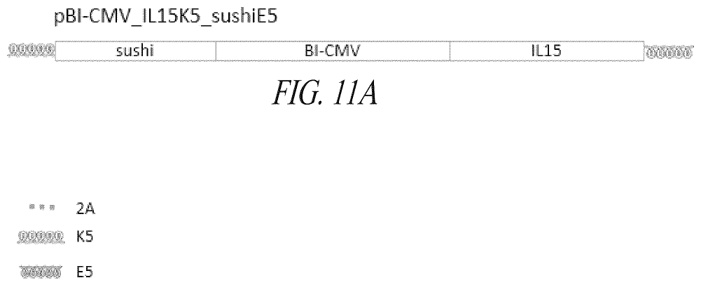

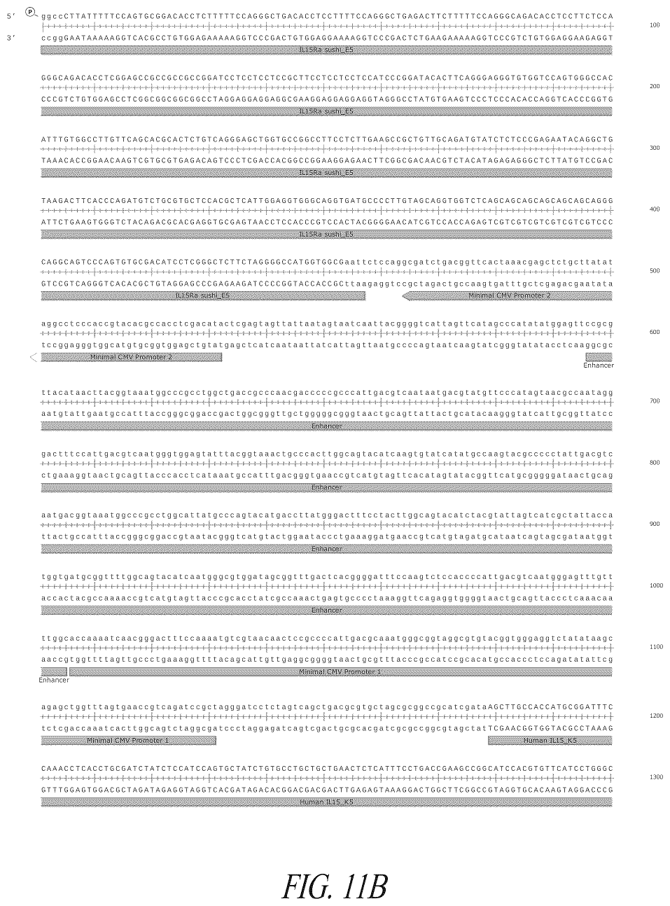

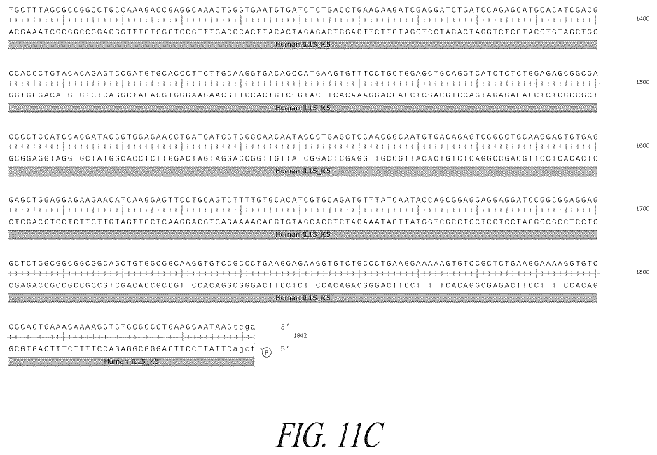

In construct 3, bi-CMV promoter drives expression of IL-15-K5 and IL-15Rα Sushi domain-E5. (FIG. 11, SEQ ID No. 559).

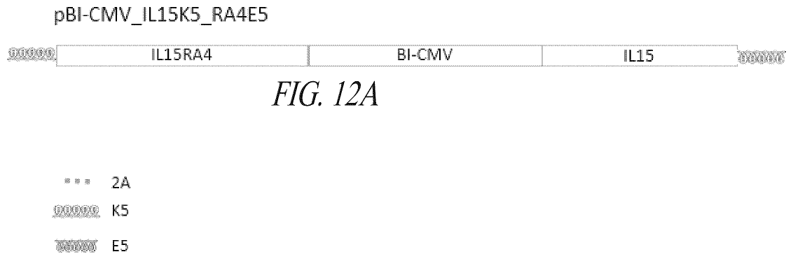

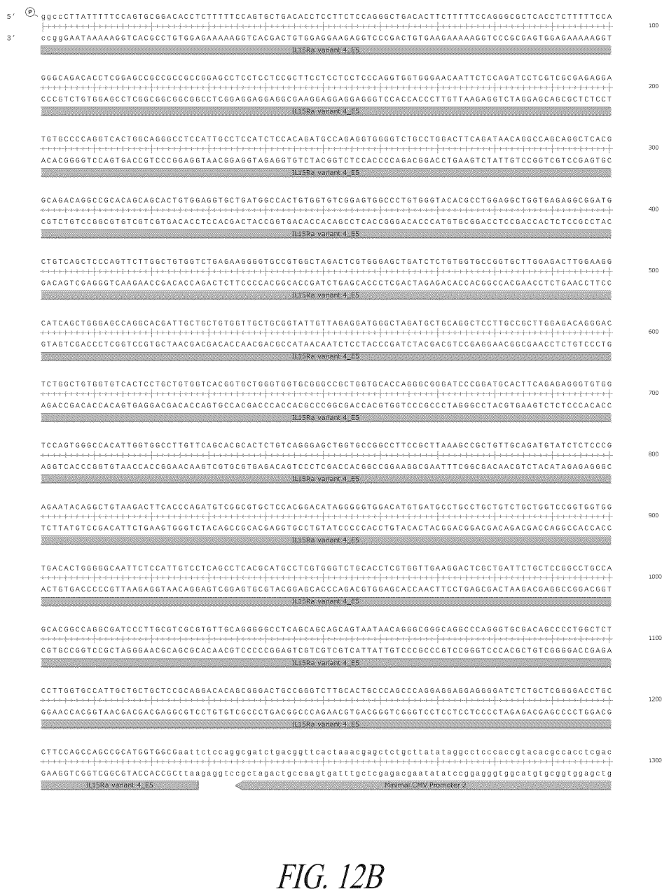

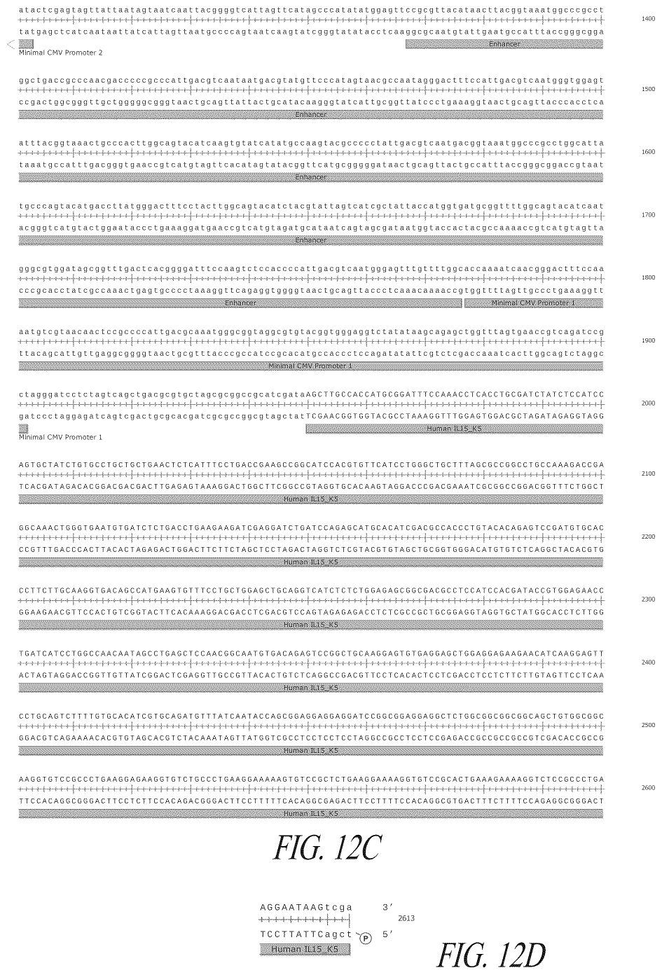

In construct 4, bi-CMV promoter drives expression of IL-15-K5 and IL-15Rα variant 4-E5 (FIG. 12, SEQ ID No. 560).

### Example 9

### Constructs Comprising IL-15 and IL-15Rα Genes Under Control of an EF1α Promoter

In this example, a variety of constructs are generated to express IL-15 and IL-15Rα in a multi-cistronic transcript under control of an EF1α promoter (SEQ ID NO: 551). IL-15 and IL-15Rα are linked by an exemplary IRES sequence (SEQ ID NO: 552).

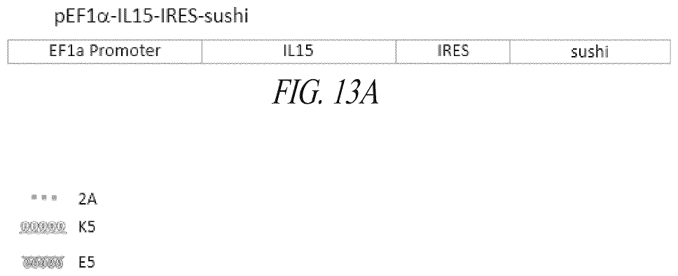

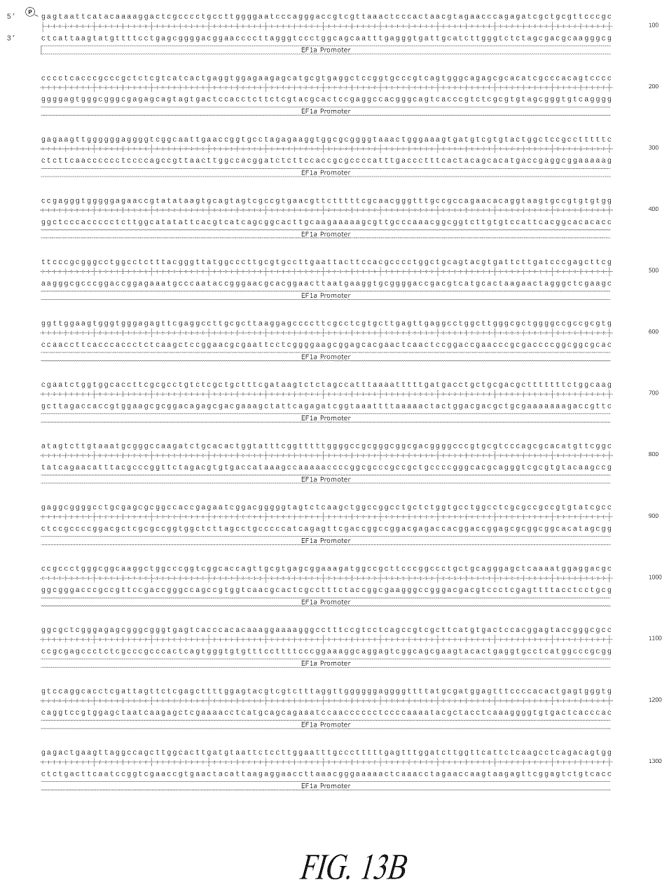

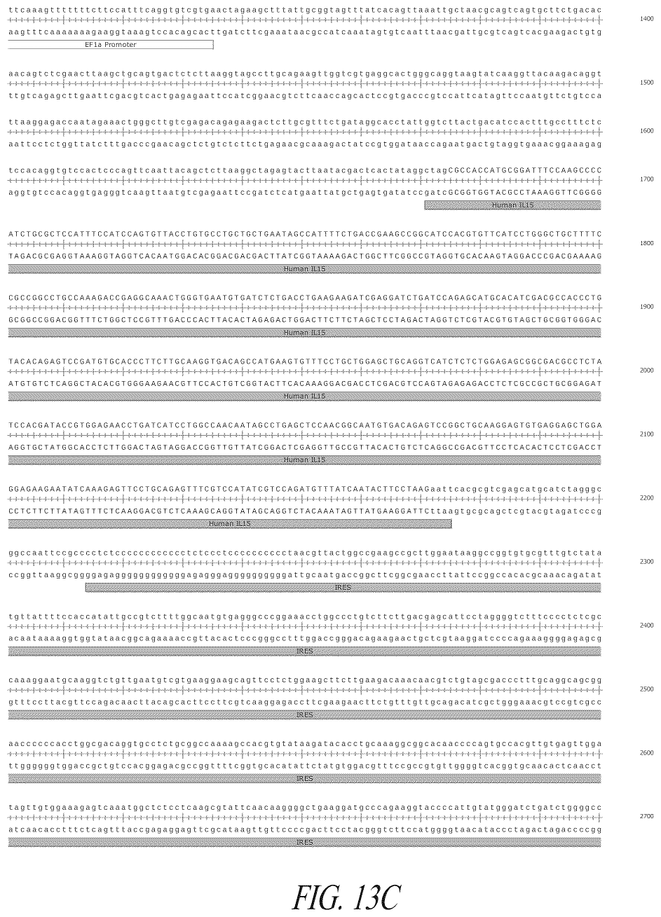

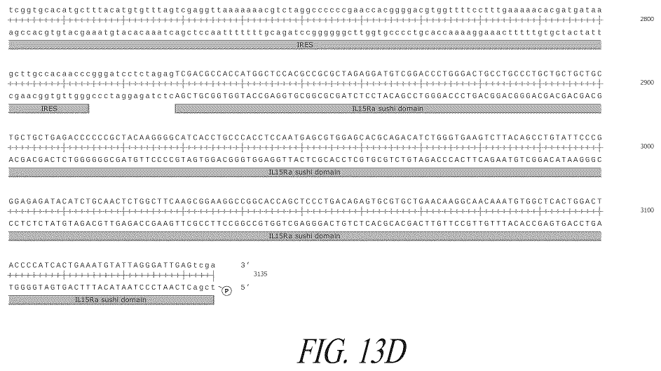

In construct 1, the EF1α promoter controls expression of IL-15-IRES-IL-15Rα Sushi domain. (FIG. 13, SEQ ID No. 561.)

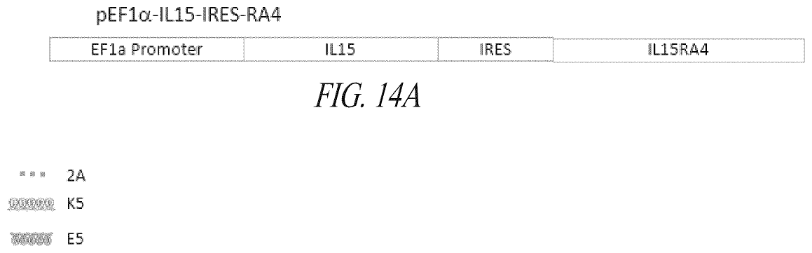

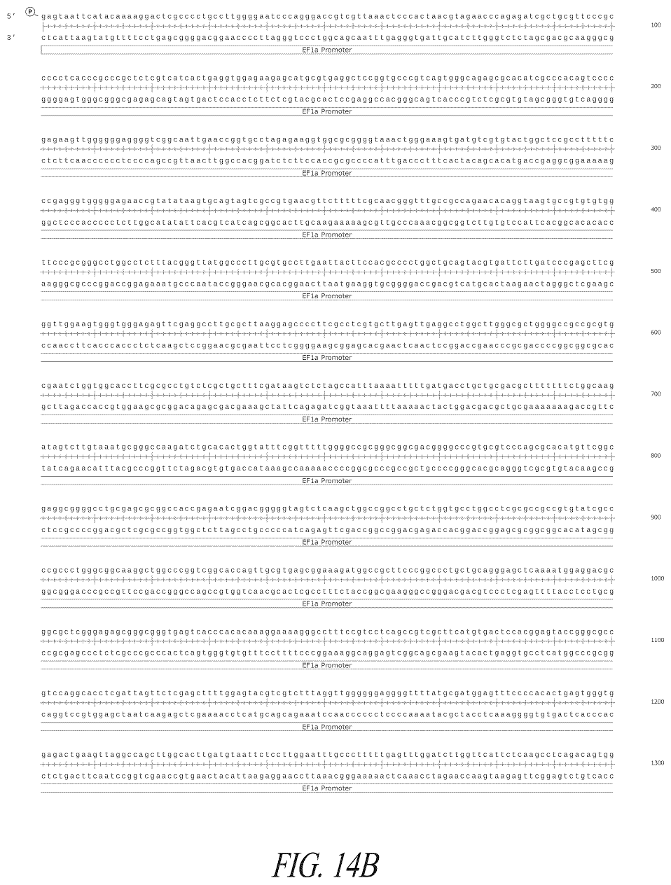

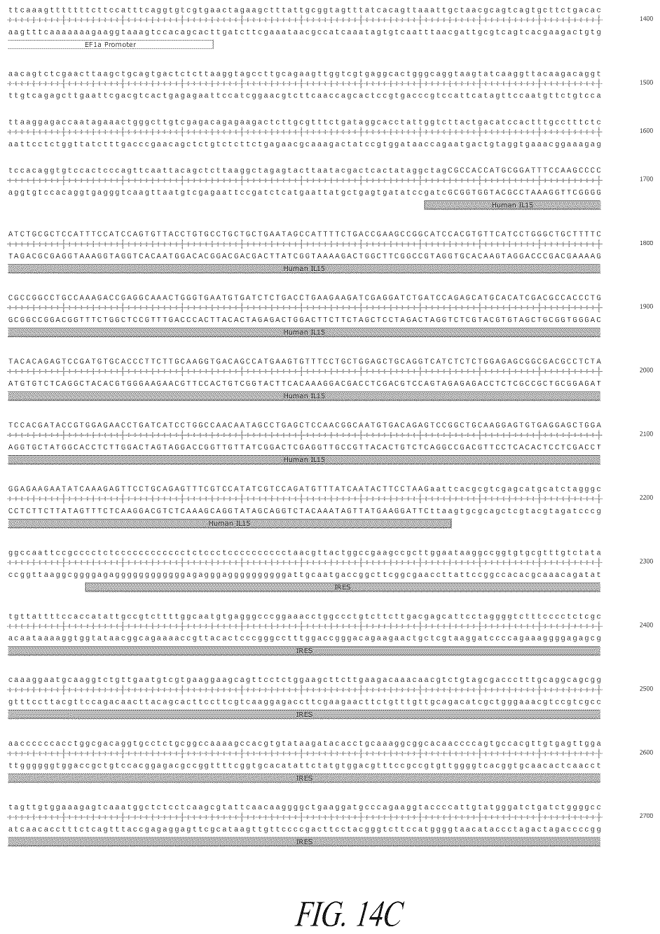

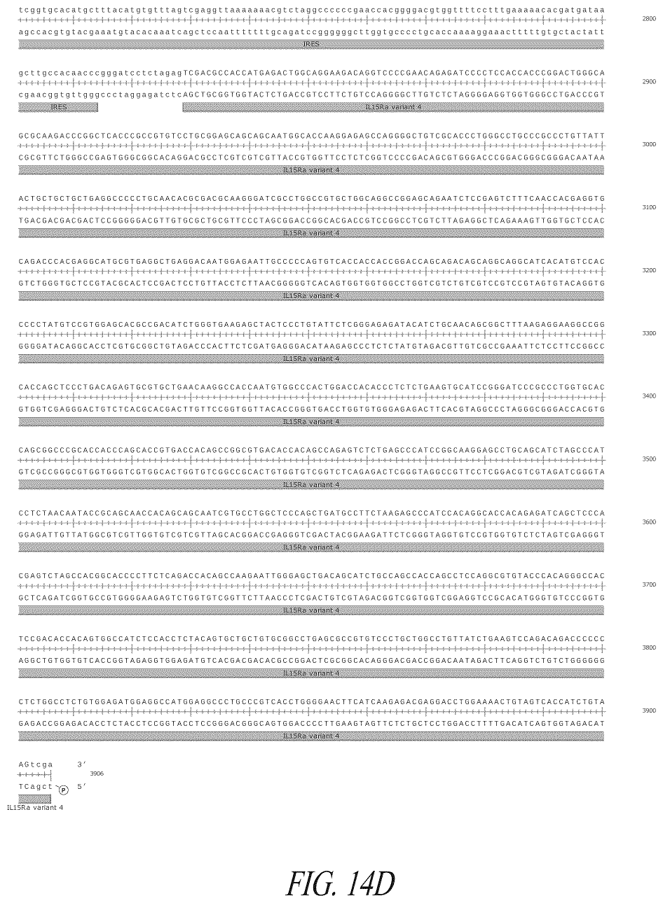

In construct 2, the EF1α promoter controls expression of IL-15-IRES-IL-15Rα variant 4 (FIG. 14, SEQ ID No. 562.)

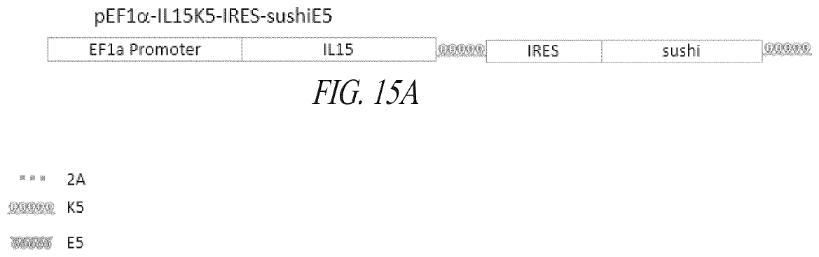

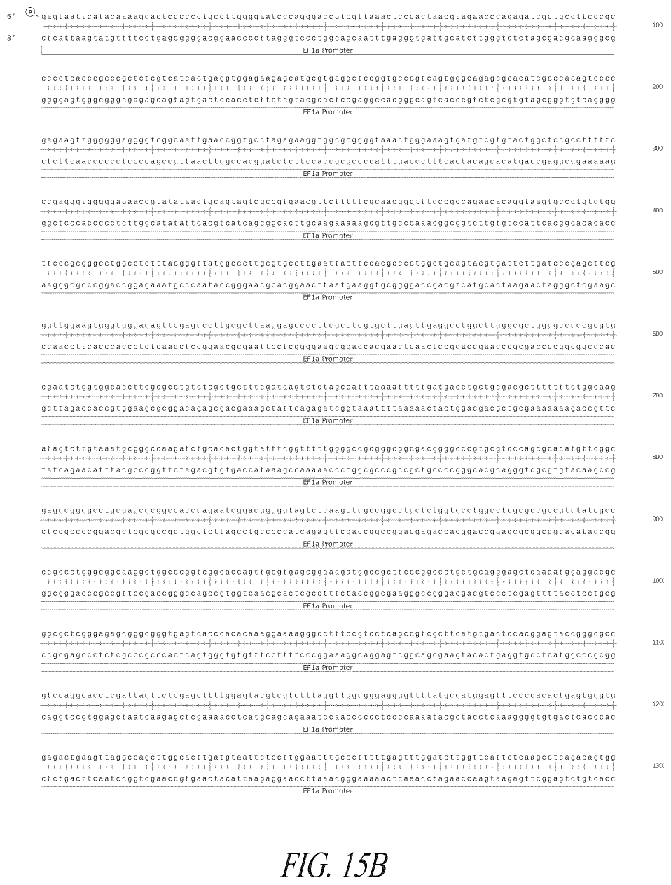

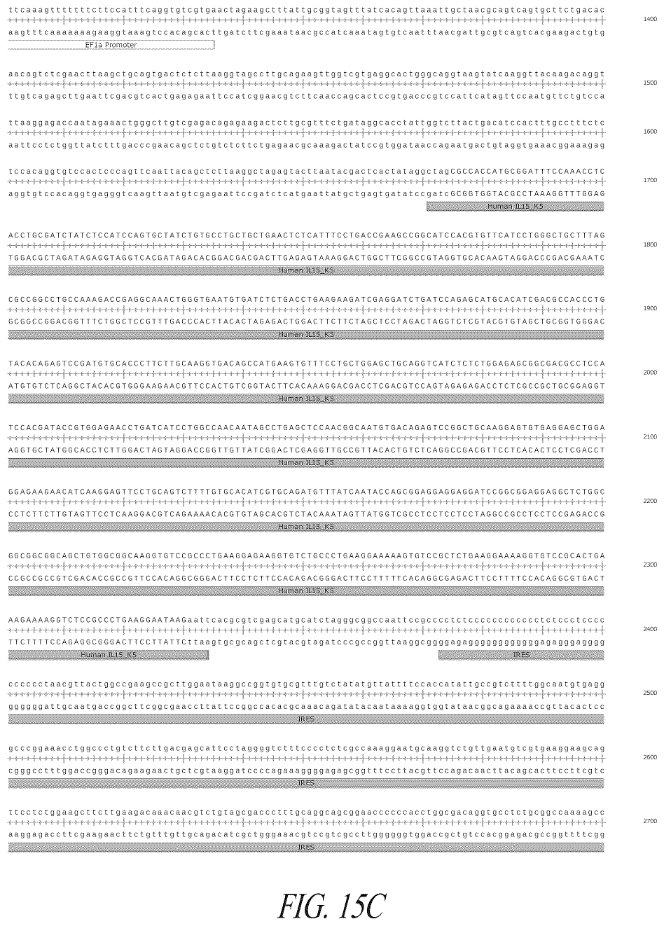

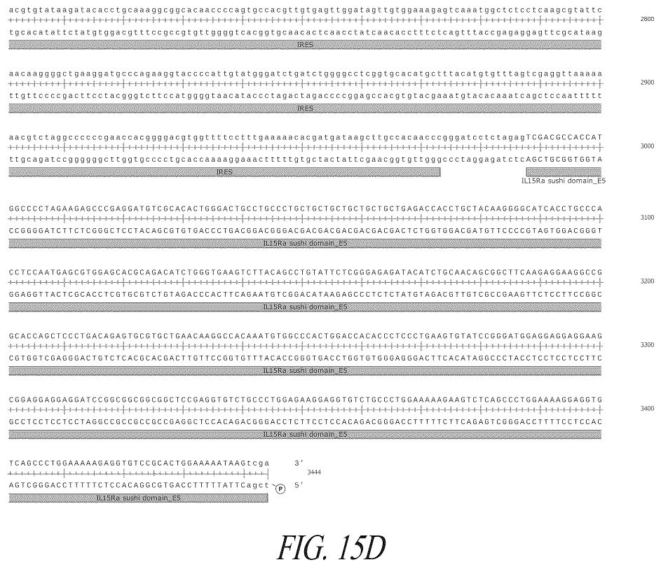

In construct 3, the EF1α promoter controls expression of IL-15K5-IRES-IL-15Rα Sushi domainE5. (FIG. 15, SEQ ID No. 563.)

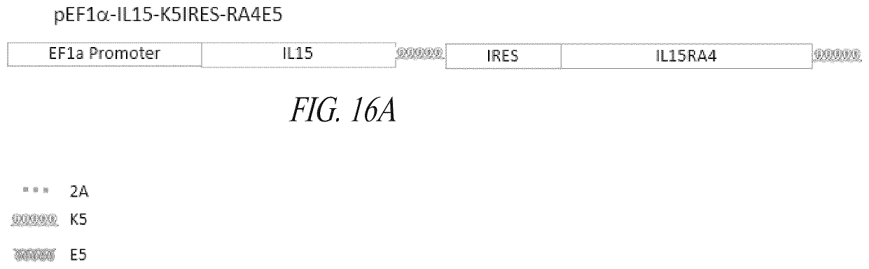

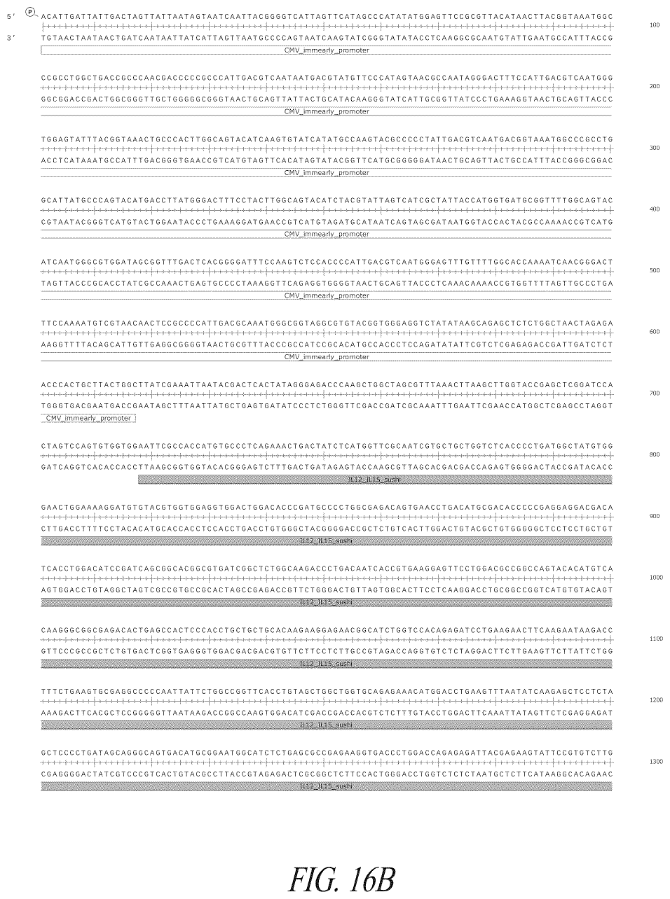

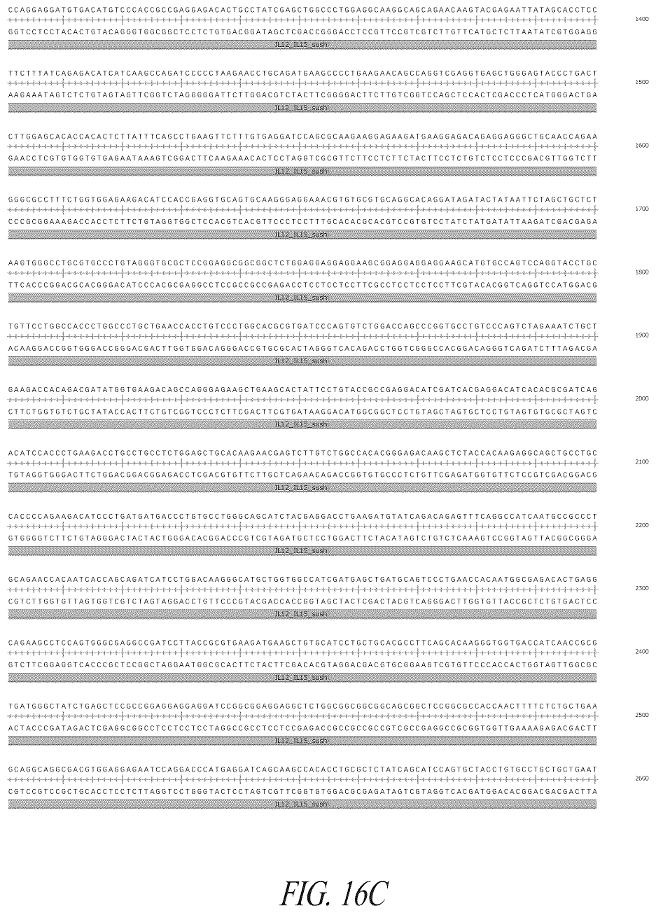

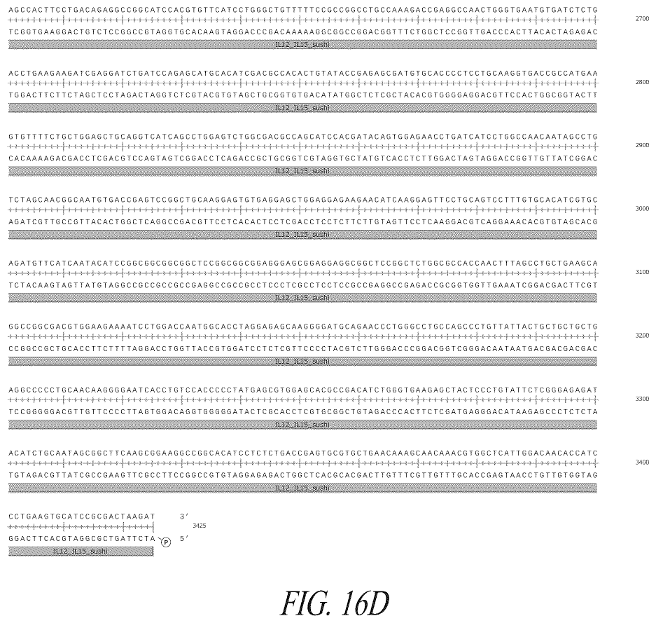

In construct 4, the EF1α promoter controls expression of IL-15K5-IRES-IL-15Rα variant 4E5. (FIG. 16, SEQ ID No. 564.)

### Example 10

### Constructs Comprising IL-12, IL-15 and IL-15Rα Genes Under Control of a CMV Promoter

In this example, a variety of constructs are generated to express IL-12, IL-15 and IL-15Rα in a multi-cistronic transcript under control of a CMV promoter. IL-12, IL-15 and IL-15Rα are linked by an exemplary p2A sequence (SEQ ID NO: 554).

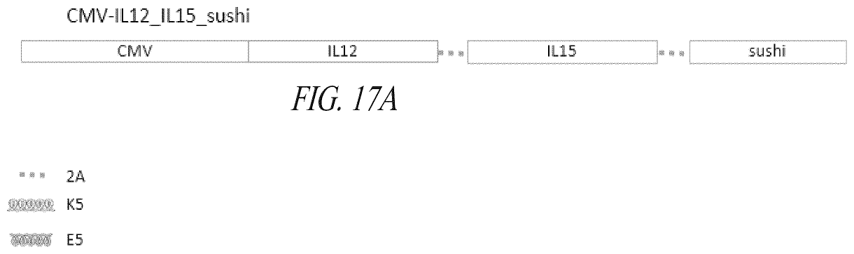

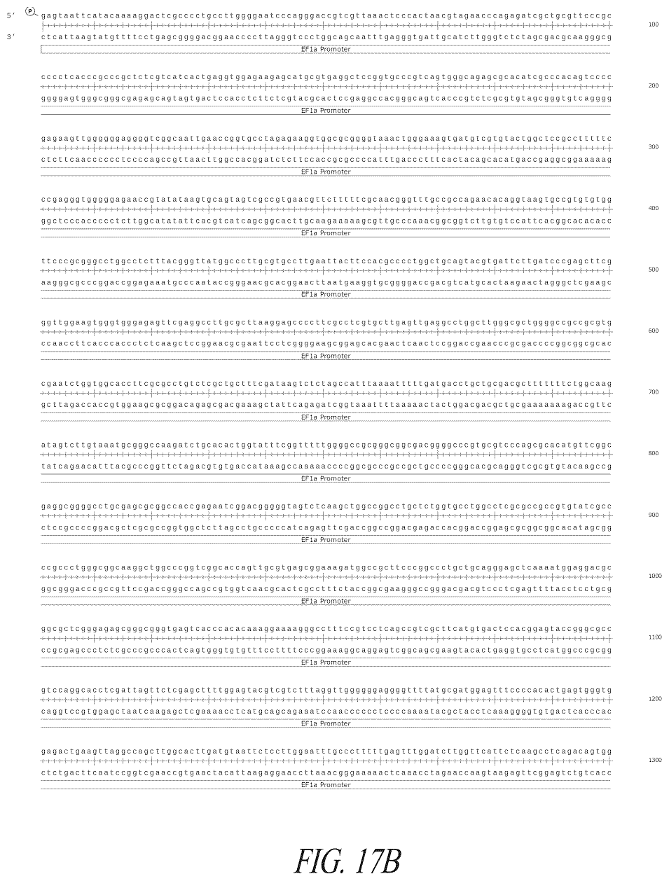

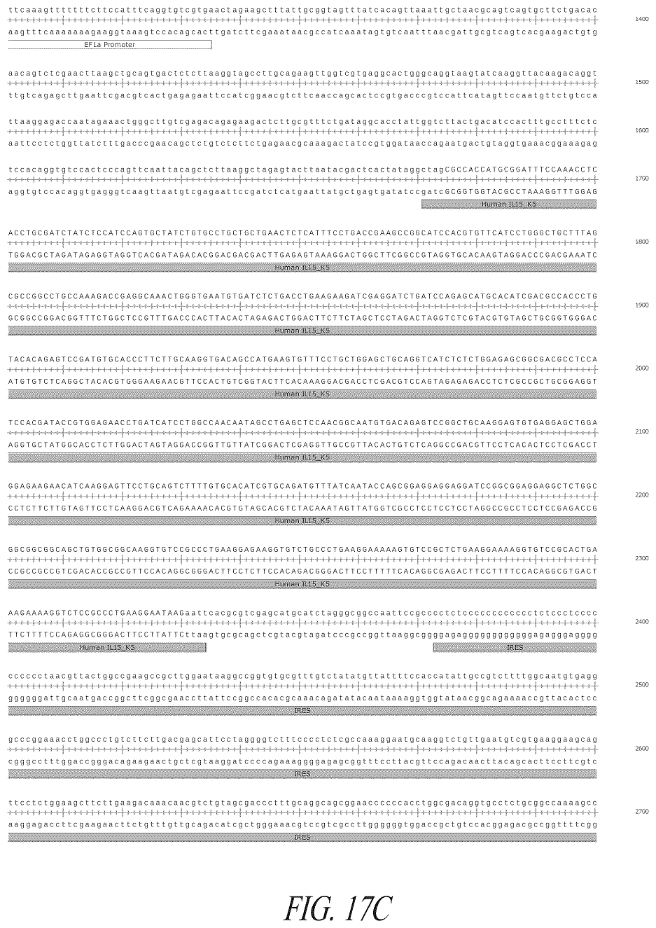

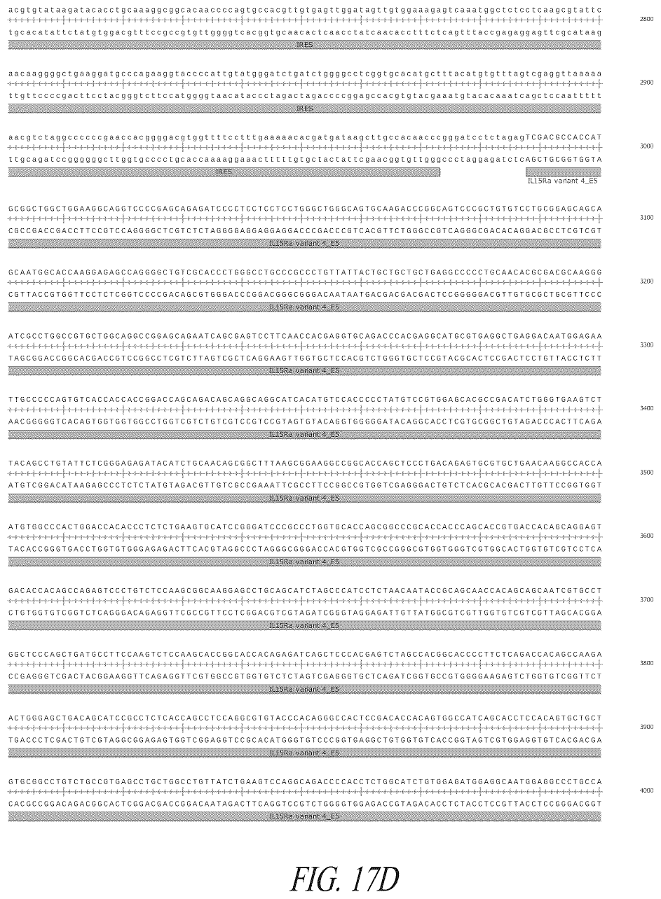

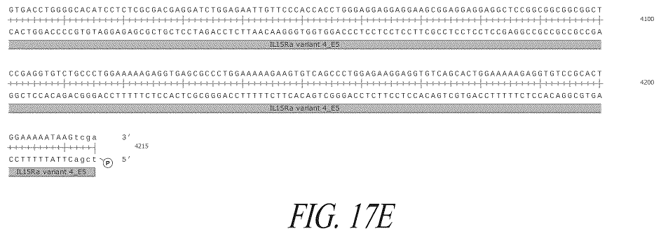

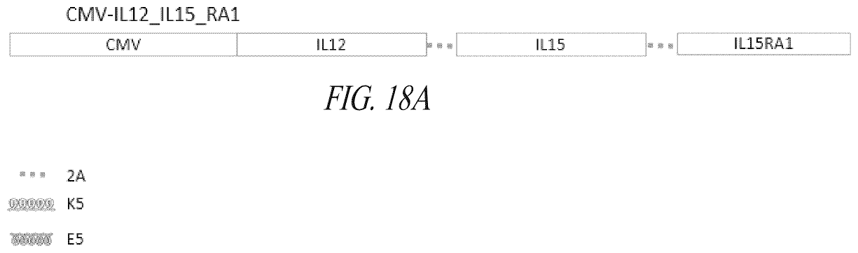

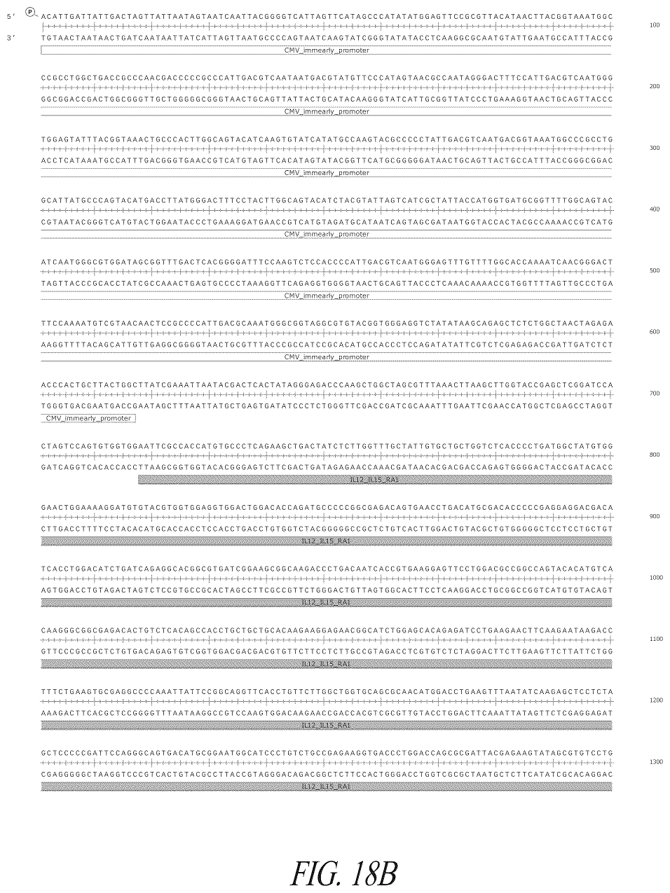

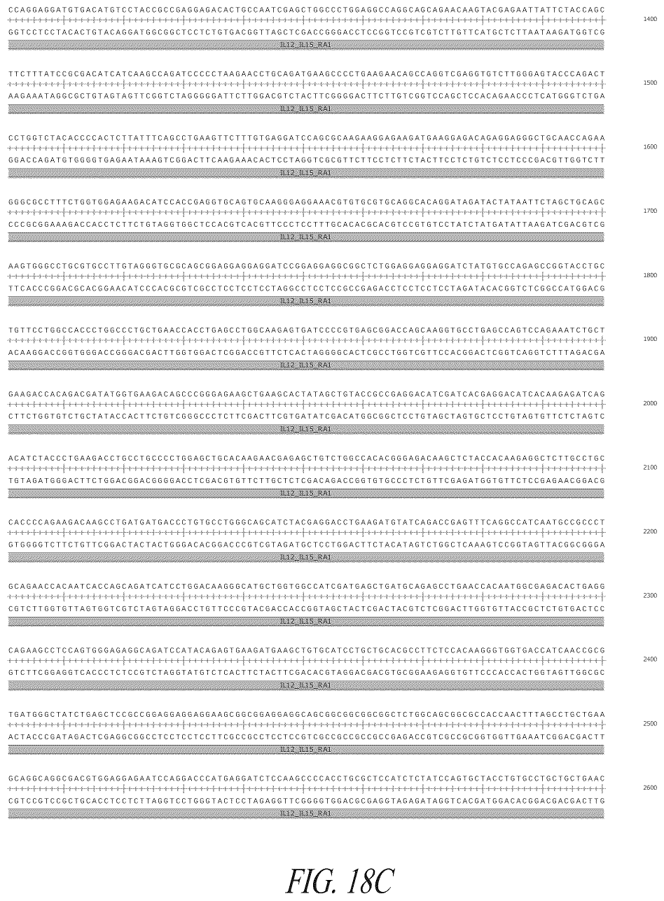

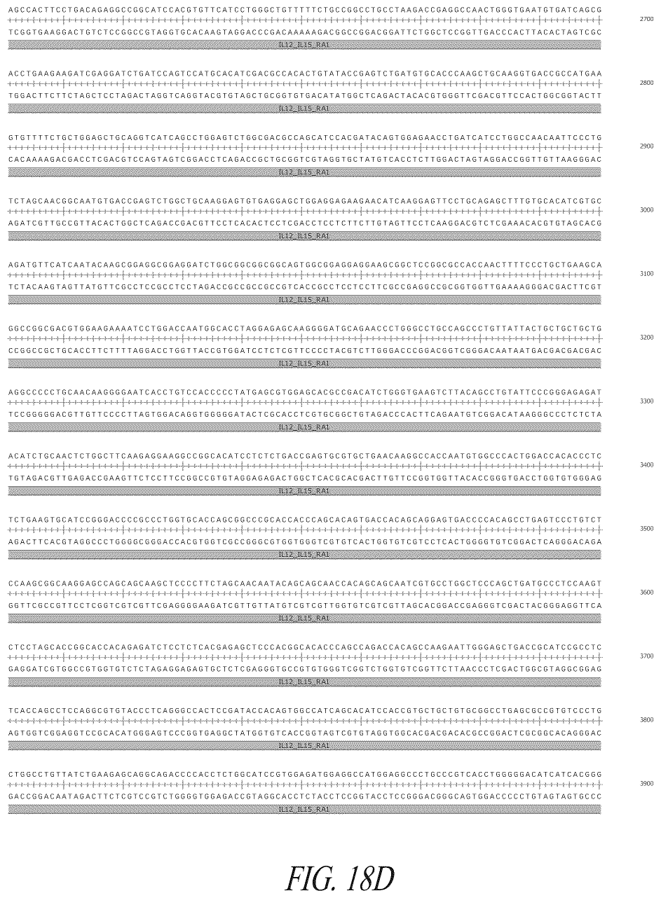

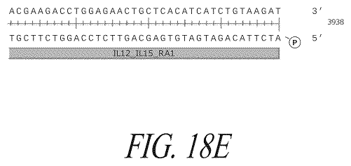

In construct 1, the CMV promoter (SEQ ID NO: 553) controls expression of IL-12-p2A-IL-15-p2A-IL-15Rα Sushi domain. (FIG. 17, SEQ ID Nos. 565, 569.)

In construct 2, the CMV promoter controls expression of IL-12-p2A-IL-15-p2A-IL-15Rα variant 1 (FIG. 180, SEQ ID Nos. 566, 570.)

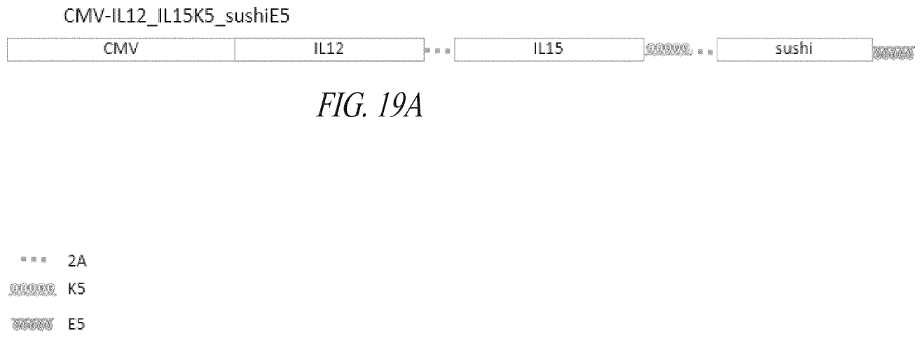

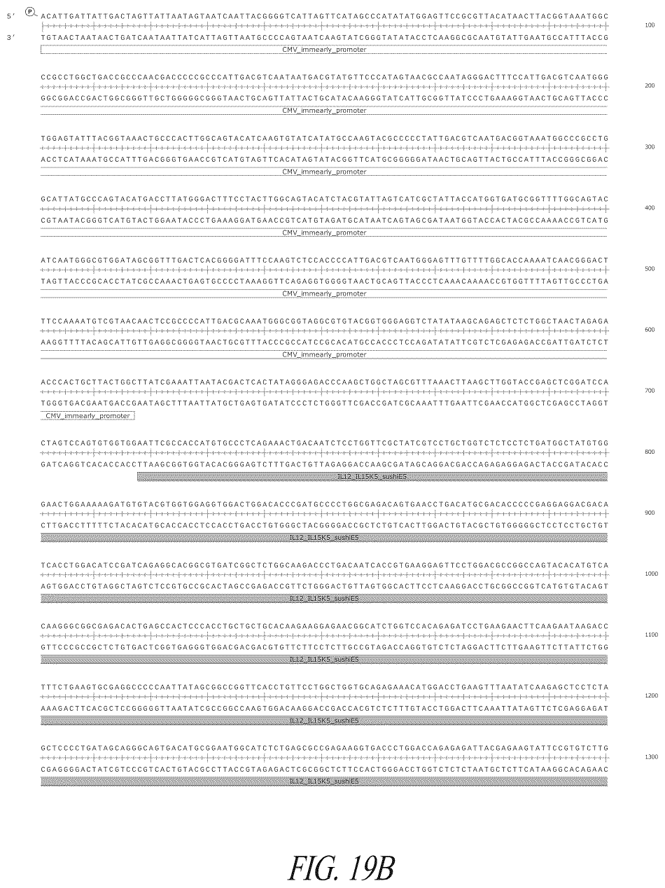

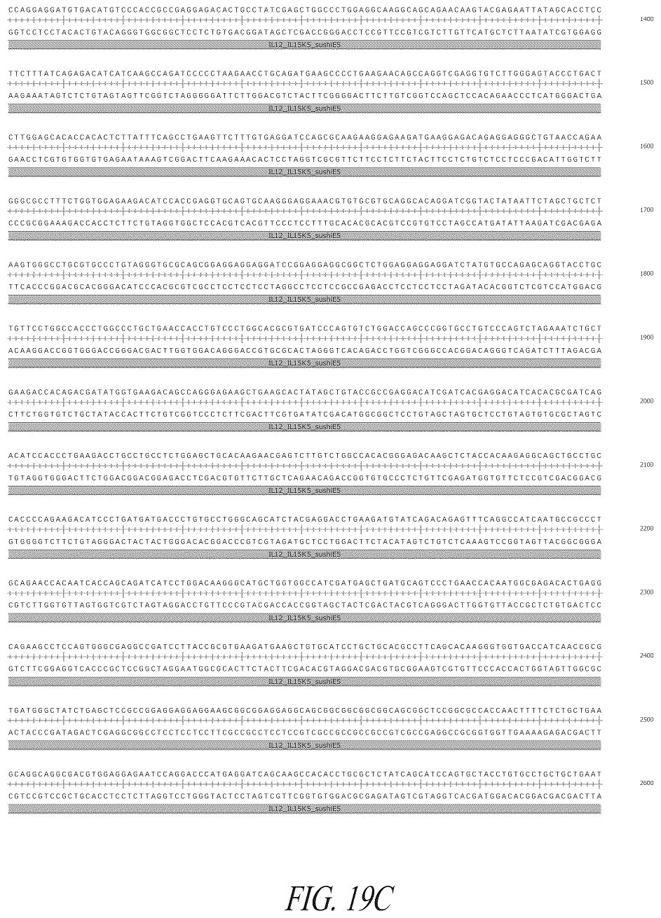

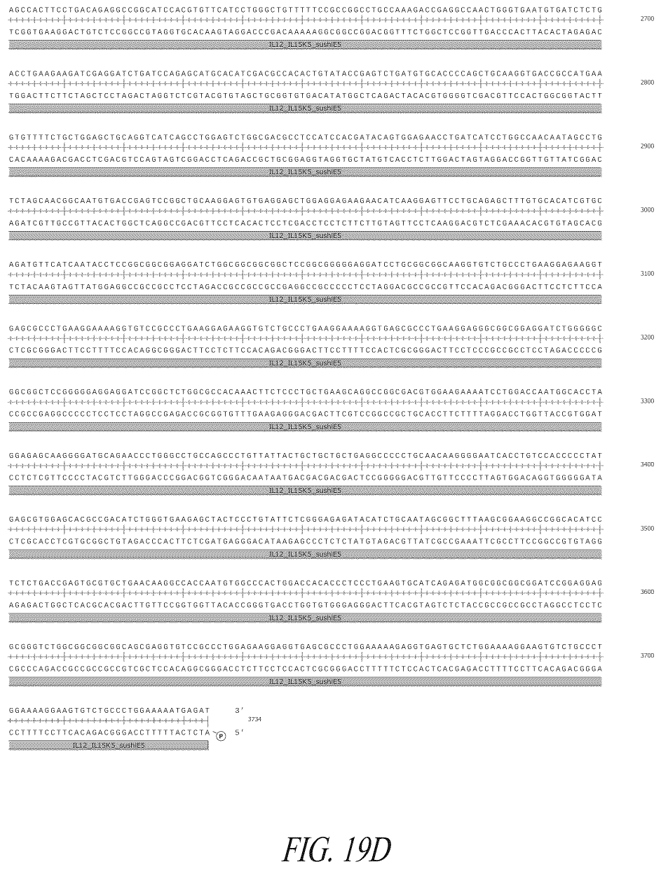

In construct 3, the CMV promoter controls expression of IL-12-p2A-IL-15K5-p2A-IL-15Rα Sushi domainE5. (FIG. 19, SEQ ID Nos. 567, 571.)

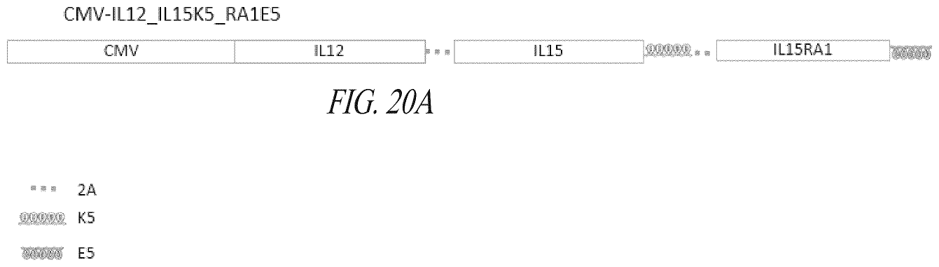

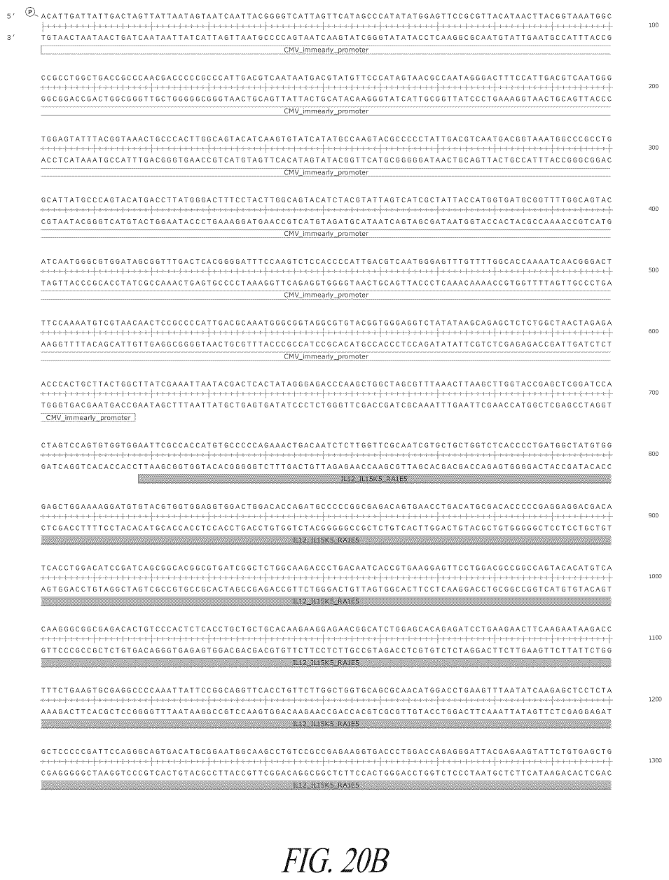

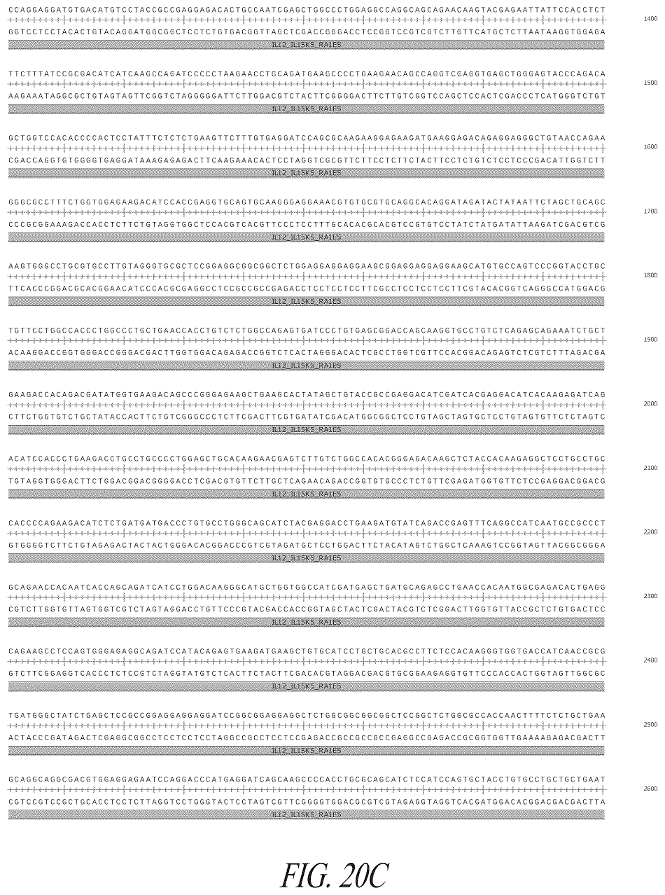

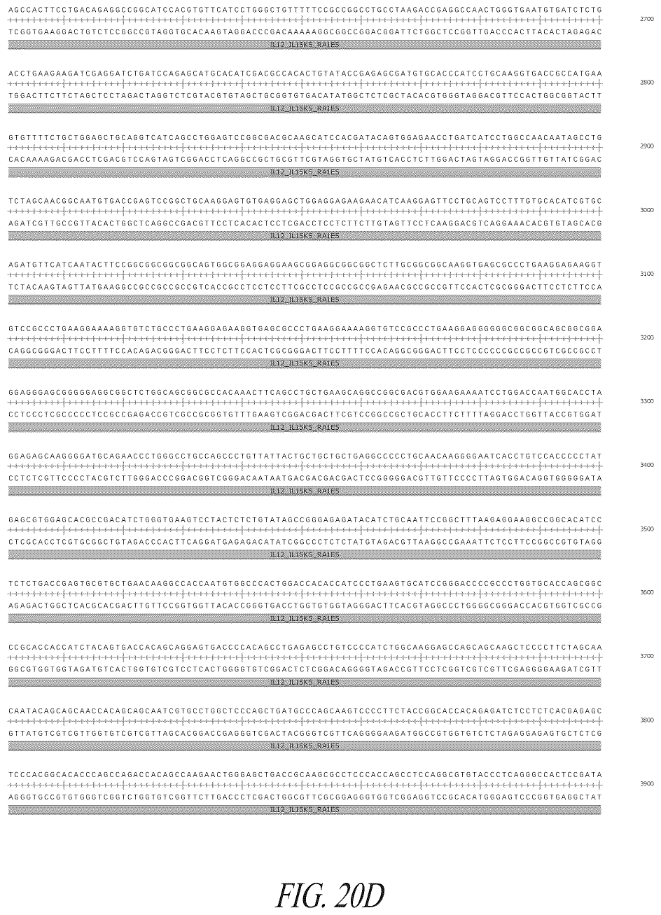

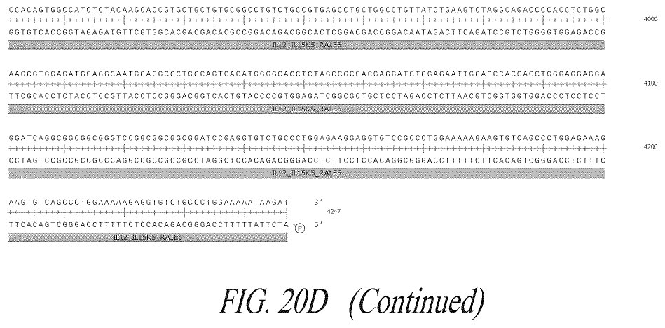

In construct 4, the CMV promoter controls expression of IL-12-p2A-IL-15K5-IRES-IL-15Rα variant 1E5. (FIG. 20, SEQ ID Nos. 568, 572.)

### Example 11

### Constructs Comprising PD-L1 Blocker Inserted Between UL3 and UL4

In this example, a construct is generated to express PD-L1 blocking peptide within the intergenic region between UL3 and UL4 between bases 829 and 830. In SEQ ID NO: 556, bases 1-675: UL3 coding sequence; bases 676-829: region between UL3 and UL4 and upstream of the PD-L1 blocker cassette; bases 830-833: region between UL3 and UL4 and downstream of the PD-L1 blocker cassette; bases 834-1433: UL4 coding sequence.

### Example 12

### Inhibition of Human PD-L1 Binding to PD-1 by Blocking Peptides

Recombinant human PD-L1 Fc protein was coated to the bottom of 96-well flat-bottom plate at 4° C. for overnight. After overnight plate coating, different PD-L1 blockers were added into each well of plate and incubated at room temperature for 2 hours before addition of recombinant human PD-1 Fc protein. Biotinylated anti-human IgG antibody and Streptavidin-HRP were subsequently added into each well, and the binding of human PD-1 to PD-L1 was detected by adding TMB substrate. Color development was measured by microplate reader at 450 nm wavelength. Percentage of inhibition was calculated by comparing to no synthesized peptide control. FIG. 21 shows the percentage inhibition by peptides ET, ET+TF, YT, YT+TF, TW, TW+TF, WT, WT+TF and TF at two different concentrations (3 and 10 μM). At 10 μM, inhibition ranged from about 22% to about 48%.

### Example 13

### Blocking PD-L1 Binding by Blocking Peptides Enhances Cytotoxicity Against Tumour Cells

Human peripheral blood mononuclear cells (PBMCs) were stimulated with anti-CD3 antibody plus human IL-2 for 24 hours and were subsequently incubated with different synthesized PD-L1 blockers and calcein-AM labelled target cells for 4 hours. Cell cultured supernatants were harvested after 4-hour incubation and released calcein-AM fluorescence was measured by microplate reader. Percentage of cytotoxicity was calculated based on the following formula: [(sample reading−minimum release)/(Maximum release−minimum release)]×100.

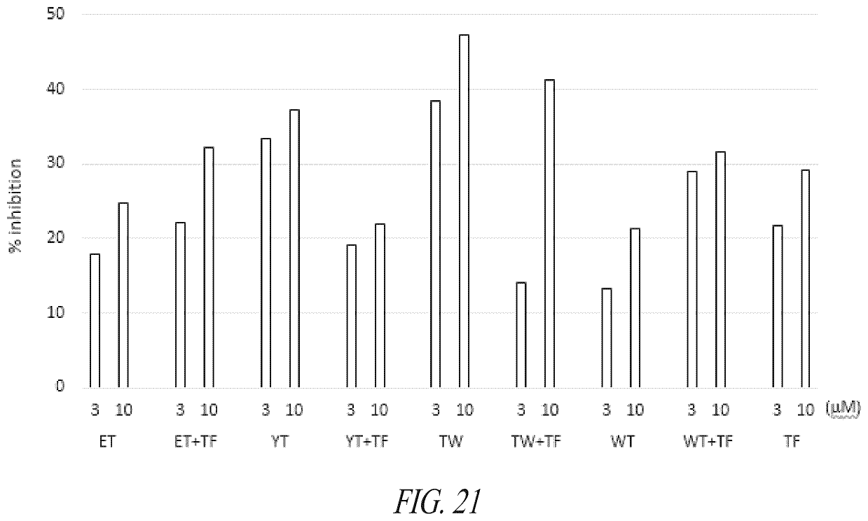

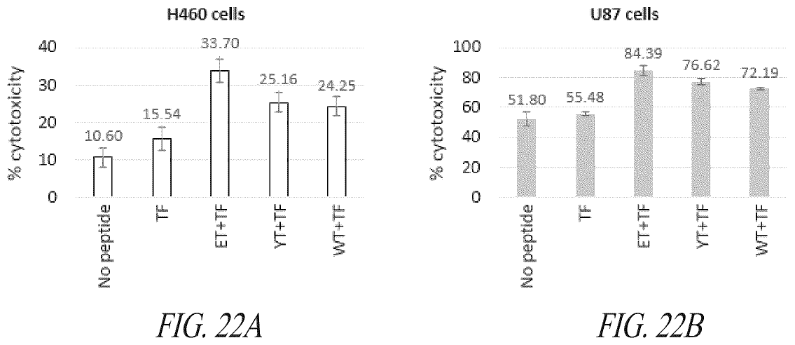

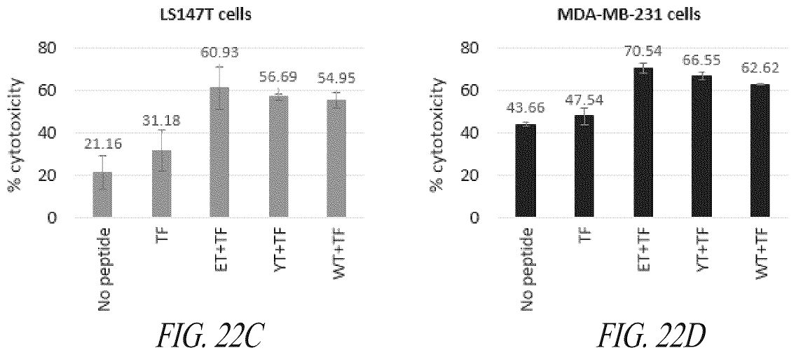

FIGS. 22A-22D shows results for four different tumour cells: H460, U87, LS147T and MDA-MB-231 cells. Increase of cytotoxicity was statistically significant for all peptides except for TF on some tumour cells.

### Example 14

### Synergistic Effect of IL-12 and IL-15 on Cytokine Production

Human PBMCs were incubated with medium control, IL-12 alone, IL-15RA alone, or combined IL-12, IL-15, and IL-15Rα1 plus neutralizing anti-IL-12 or anti-IL-15 antibody for 48 hours. Cell cultured supernatants were harvested to measure the production of human IFNγ and TNFα by ELISA.

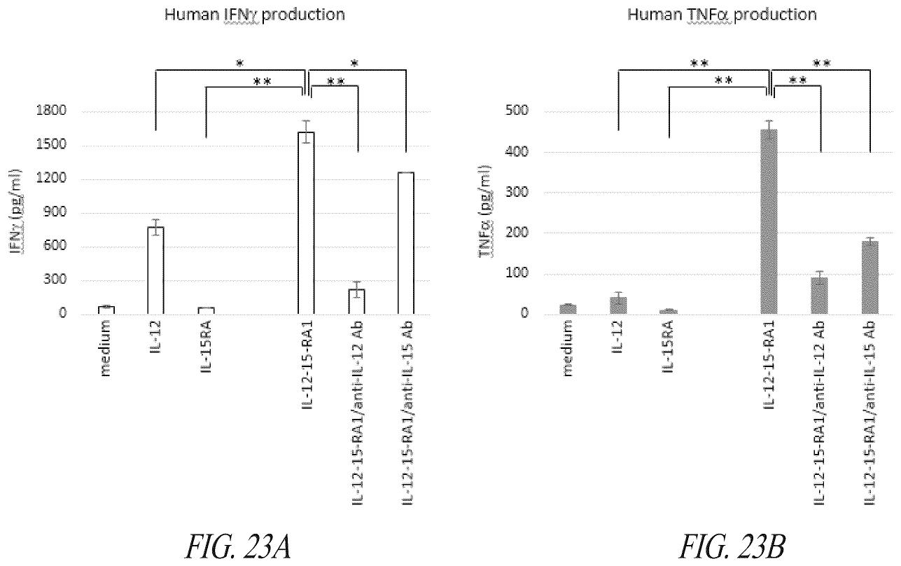

FIGS. 23A and 23B shows results of cytokine production. The combination of IL-12 and IL-15Rα1 caused a statistically significant increase of cytokines human IFNγ and TNFα. Production was inhibited with anti-IL-12 antibody.

### Example 15

### Synergistic Effect of IL-12 and IL-15 on Cytotoxicity Against Tumour Cells

Human PBMCs were co-incubated with tumour target cells and medium control, IL-12 alone, IL-15RA alone, or combined IL-12, IL-15, and IL-15RA1 plus neutralizing anti-IL-12 or anti-IL-15 antibody for 24 hours. Cell cultured supernatants were harvested to measure cytotoxicity by LDH assay. Percentage of cytotoxicity was calculated based on the following formula: [(sample reading−minimum release)/(Maximum release−minimum release)]×100.

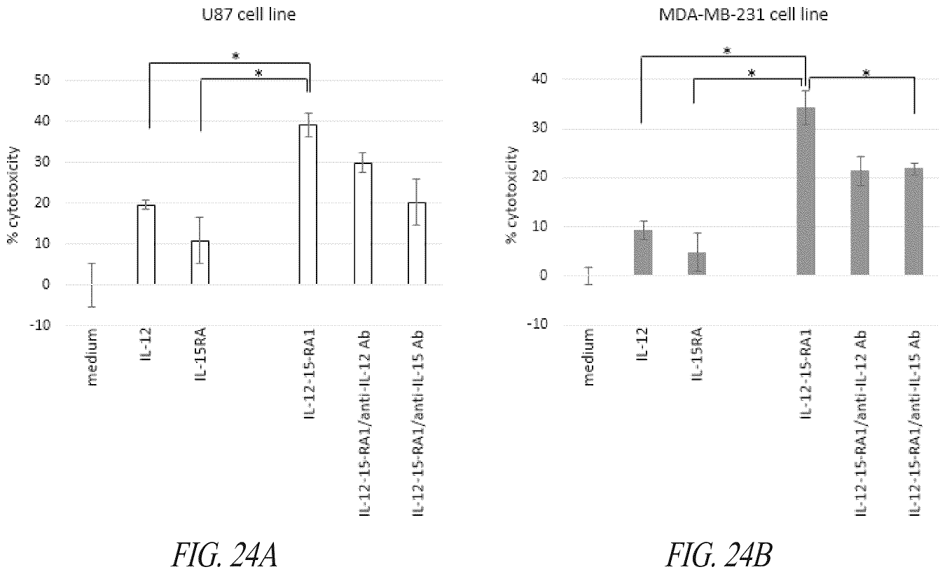

FIGS. 24A and 24B shows that IL-12 and IL-15 together increased cytotoxicity in a statistically significant manner. On the MDA-MB-231 cell line, the addition of anti-IL-12 or IL-15 antibody significantly reduced the effect.

### Example 16

### In Vitro Efficacy of Viruses VG161-pLBh and VG161-15h

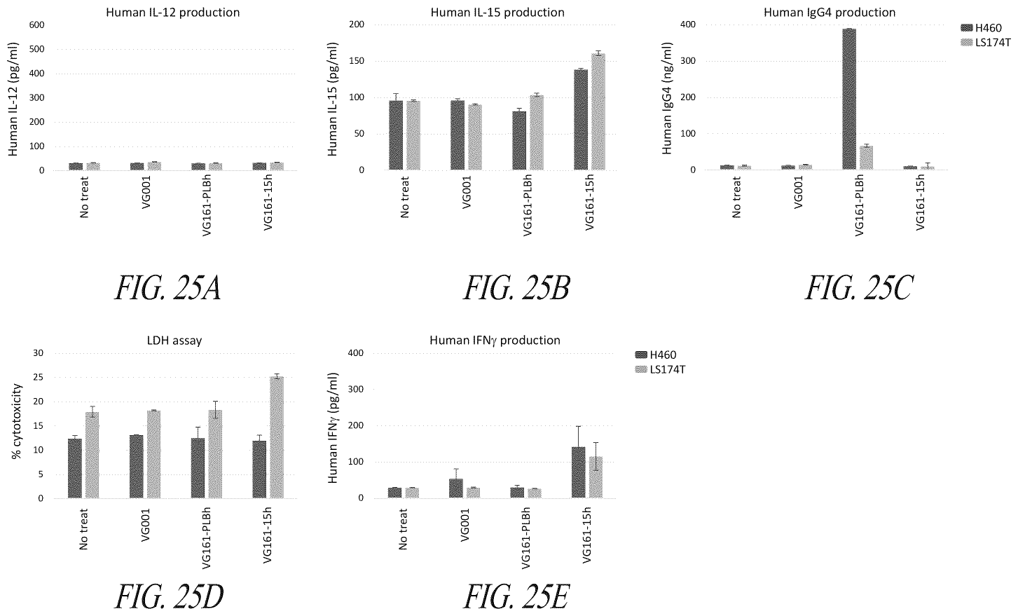

In this example, 3×10⁴ H460 or LS174T tumour cells were seeded into each well of 96-well plate and cultured at 37° C. for overnight. Next day, seeded cells were infected with VG001 backbone, VG161-PLBh, or VG161-15h virus (MOI=1) for 24 hours and the productions of human IL-12, human IL-15, and human IgG4 were assessed (FIG. 25A-C). 3×10⁵ human PBMCs were subsequently added into the culture and co-incubated for 24 hour to assess cytotoxicity by LDH assay (FIG. 25D) or 48 hours for human IFNg production by ELISA (FIG. 25E). For the cytotoxicity assay, percentage of cytotoxicity was calculated based on the following formula: [(actual reading−minimum release)/(Maximum release−minimum release)]×100%. Supernatant harvested from tumour cells incubated with medium only was used as minimum release, and supernatant harvested from tumour cells incubated with lysis buffer was used as maximum release.

### Example 17

### In Vitro Efficacy of Various Constructs

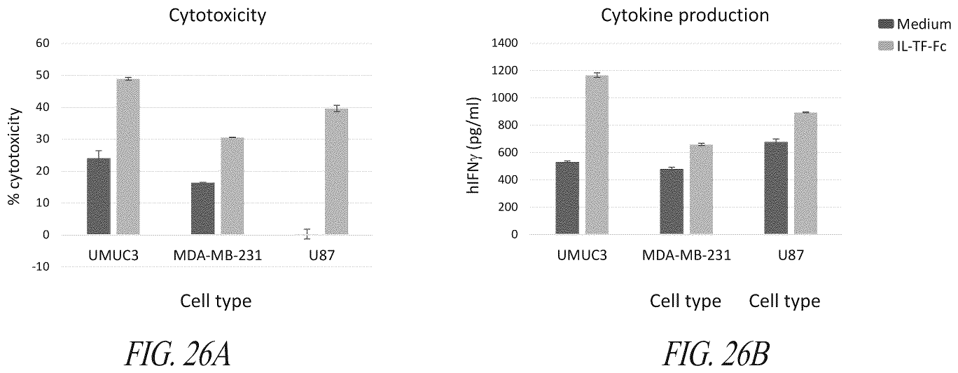

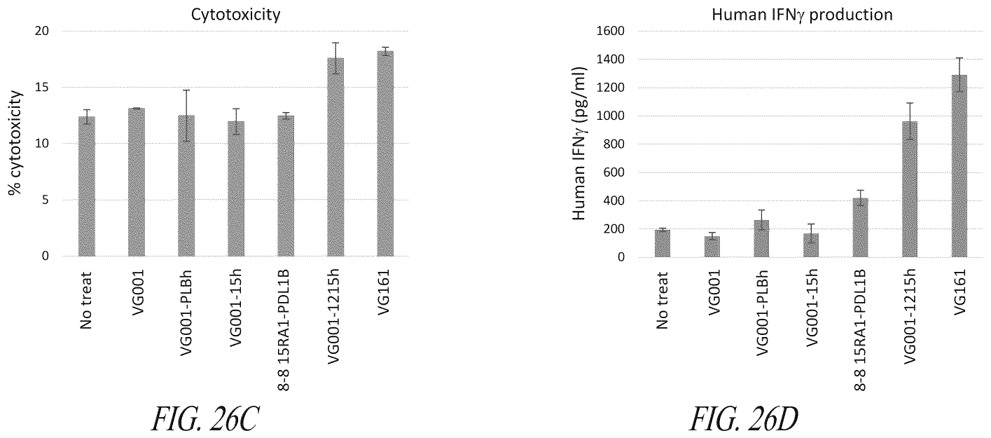

FIGS. 26A-26D show results of in vitro assays for various constructs.

FIGS. 26A-26B show results of cell transfection with IL-TF-Fc plasmid carrying IL-12, IL-15, and PD-L1 blocker. In FIGS. 26A-26B, different tumour cell lines were transfected with IL-TF-Fc plasmid DNA for 24 hours, and human PBMCs were subsequently added into the culture. Cell supernatants were harvested after 24 hours for quantification of cytotoxicity by LDH assay (FIG. 26A), and after 48 hours for detection of human IFNg production by ELISA assay (FIG. 26B).

FIGS. 26C-26D show results of cell infection with a variety of mutant viruses including hVG161. Virally encoded IL12, IL15, and PD-L1 blocker synergistically enhance IFNg production and cytotoxicity. H460 tumour cells were seeded into each well of a 96-well plate and cultured at 37° C. for overnight. Next day, seeded cells were infected with the indicated viruses at MOI=1 for 24 hours. Human PBMCs were subsequently added into the culture and co-incubated for 24 hour to assess cytotoxicity by LDH assay (FIG. 26C) or 48 hours for human IFNg production by ELISA (FIG. 26D). For cytotoxicity assay, the percentage of cytotoxicity was calculated based on the following formula: [(actual reading−minimum release)/(Maximum release−minimum release)]×100%. Supernatant harvested from tumour cells incubated with medium only was used as minimum release, and supernatant harvested from tumour cells incubated with lysis buffer was used as Maximum release.

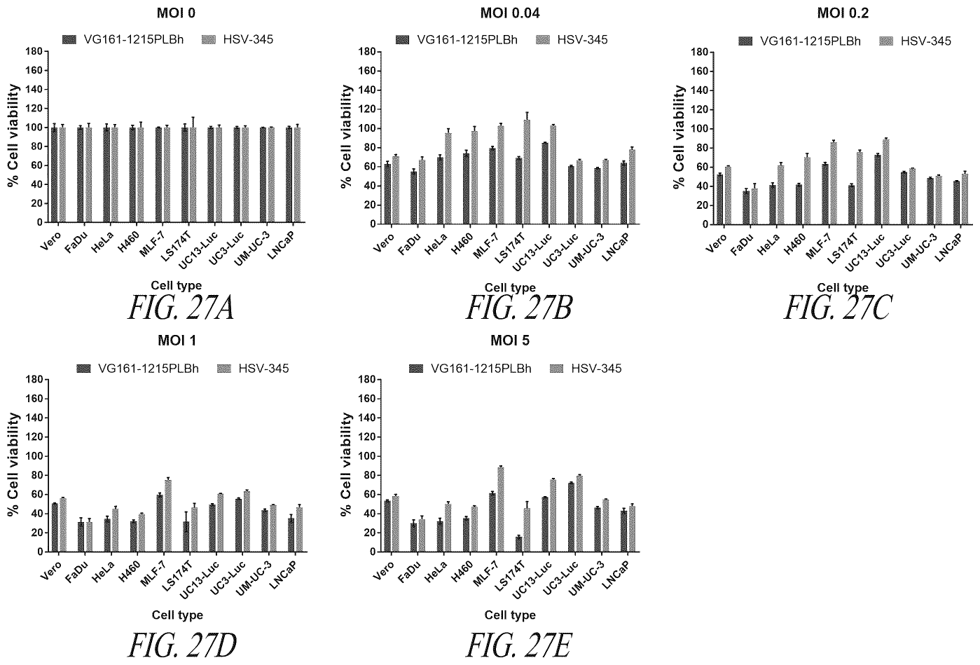

In FIGS. 27A-27E, a panel of 9 different human tumor cell lines (plus Vero cells) was infected with VG161-1212PLBh (VG161h) and HSV-345 viruses at MOI 0, 0.04, 0.2, 1, and 5. Cell viability was quantified using MTT assay at 48 hours post infection.

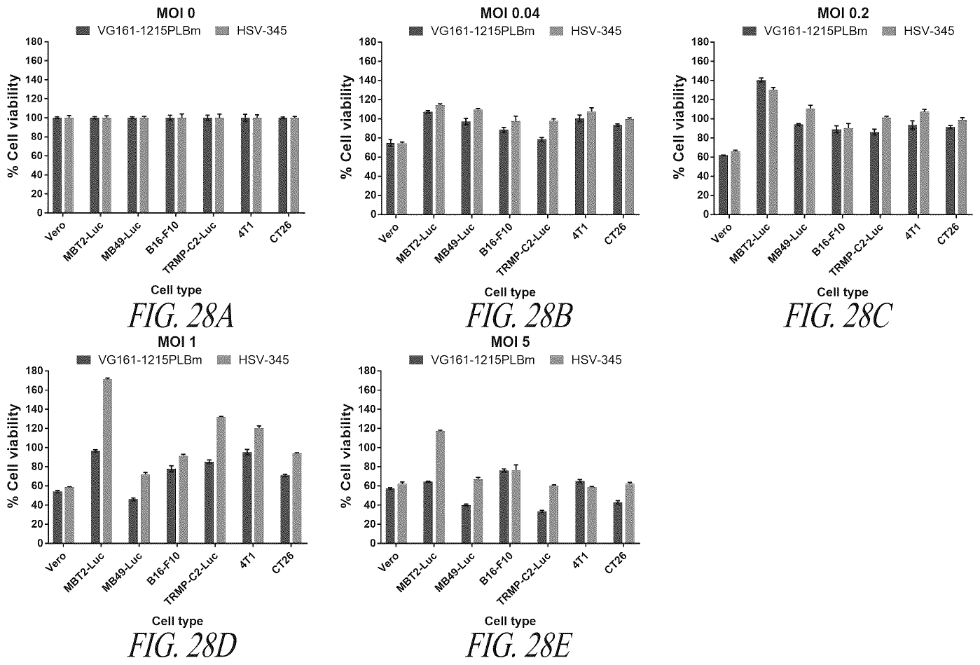

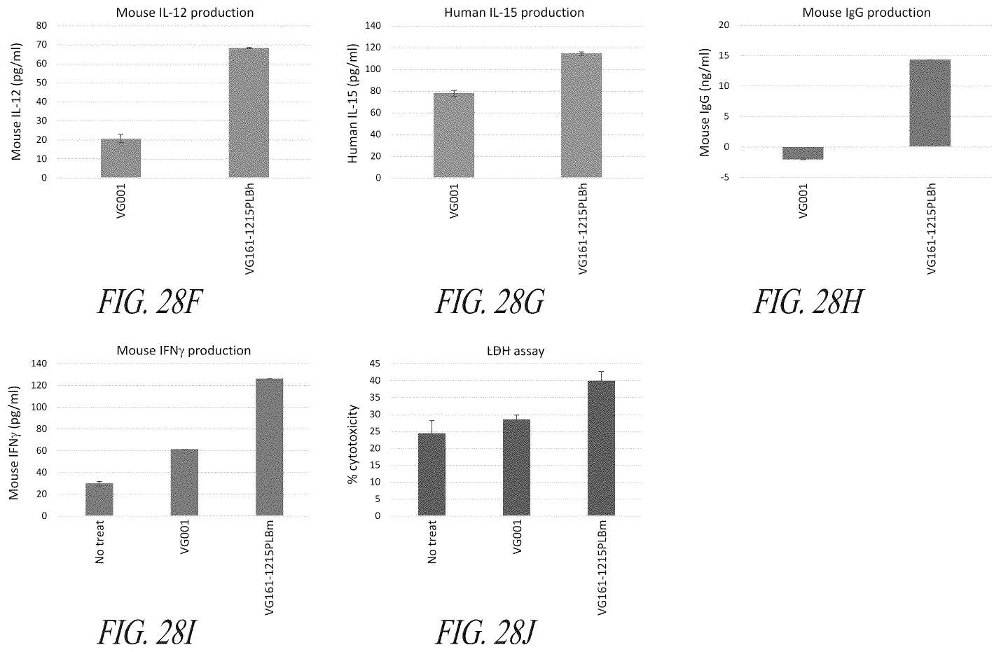

FIGS. 28A-28J show results of in vitro assays for various constructs. FIGS. 28A-28E show results of cell viability assays for mVG161 and HSV-345 on mouse tumor cell lines and Vero cell line; FIGS. 28F-28J show the characterization of transgene expression following mVG161 or VG001 infection of CT26 mouse tumor cells.

In FIGS. 28A-28E, a panel of 6 different mouse tumor cell lines (plus Vero cells) was infected with VG161m and HSV-345 viruses at MOI 0, 0.04, 0.2, 1, and 5. Cell viability was quantified using MTT assay at 48 hours post infection.

In FIGS. 28F-28J, 3×10⁴ CT26 tumour cells were seeded into each well of 96-well plate and cultured at 37° C. for overnight. Next day, seeded cells were infected with VG001 backbone or VG161-1215PLBm virus (MOI=1) for 24 hours and the production of mouse IL-12, human IL-15, and mouse IgG was assessed. 3×10⁵ splenocytes from Balb/c mouse were subsequently added into the culture and co-incubated for 24 hour to assess cytotoxicity by LDH assay or 48 hours for mouse IFNg production by ELISA. For the cytotoxicity assay, percentage of cytotoxicity was calculated based on the following formula: [(actual reading−minimum release)/(Maximum release−minimum release)]×100%. Supernatant harvested from tumour cells incubated with medium only was used as minimum release, and supernatant harvested from tumour cells incubated with lysis buffer was used as maximum release.

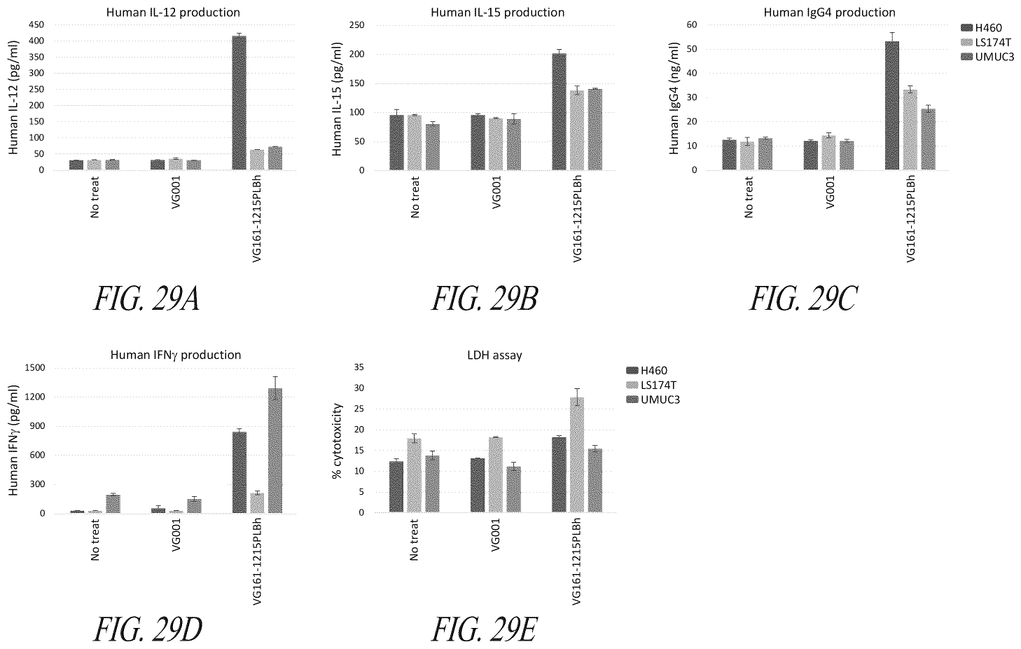

In FIGS. 29A-29E, 3×10⁴ H460, LS174T, or UMUC3 tumor cells were seeded into each well of 96-well plate and cultured at 37° C. for overnight. Next day, seeded cells were infected with VG001 backbone and VG161-1215h virus (MOI=1) for 24 hours and the productions of human IL-12, human IL-15, and human IgG4 were assessed (18R). 3×10⁵ human PBMCs were subsequently added into the culture and co-incubated for 24 hour to assess cytotoxicity by LDH assay (18S) or 48 hours for human IFNγ production by ELISA (18T). For the cytotoxicity assay, percentage of cytotoxicity was calculated based on the following formula: [(actual reading−minimum release)/(Maximum release−minimum release)]×100%. Supernatant harvested from tumor cells incubated with medium only was used as minimum release, and supernatant harvested from tumor cells incubated with lysis buffer was used as maximum release.

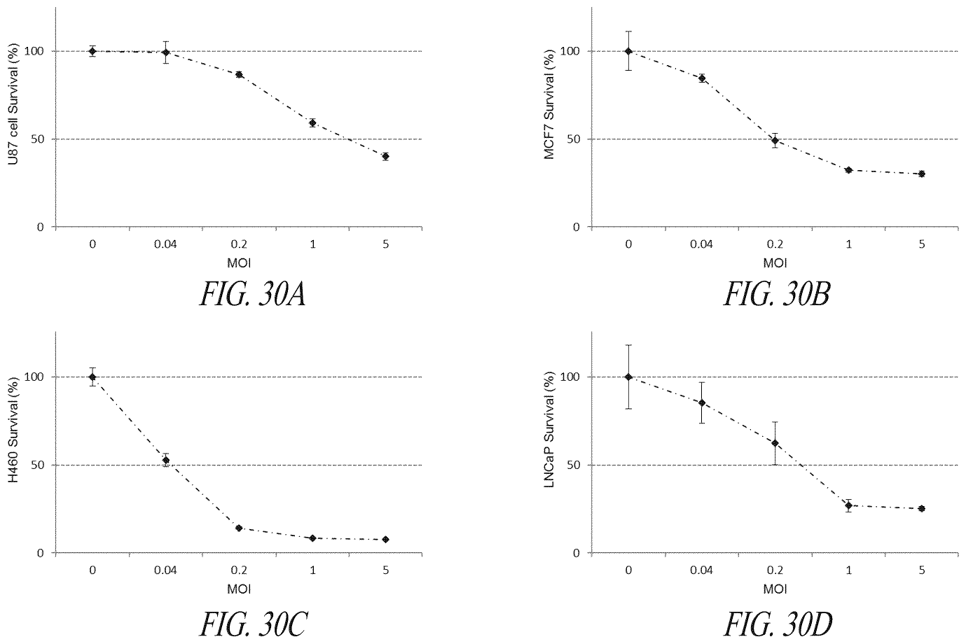

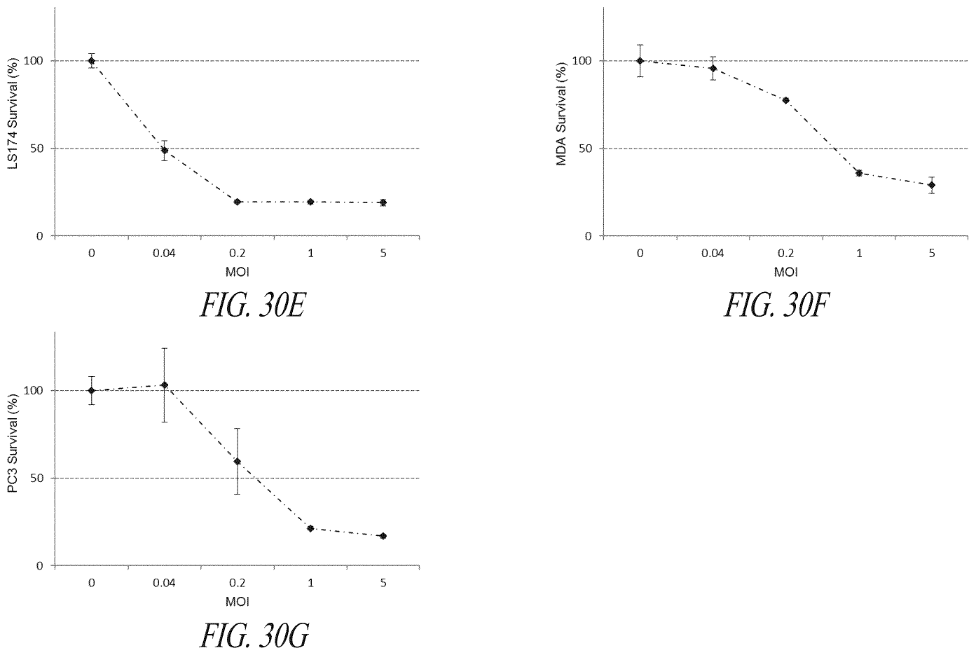

In FIGS. 30A-30G, the antitumor effect of VG161-1215PLBh (hVG161) virus was evaluated in a variety human cancer cells including U87, MCF7, H460, LNCaP, LS174T, MDA, and PC3 at 72h post infection and MOls ranging from 0 to 5. Cell survival percentage was quantified by MTT assay. The VG161-1215PLBh virus exhibits robust cell killing ability in all of the tested human tumor cell lines.

### Example 18

### In Vivo Efficacy of VG161 Viral Constructs

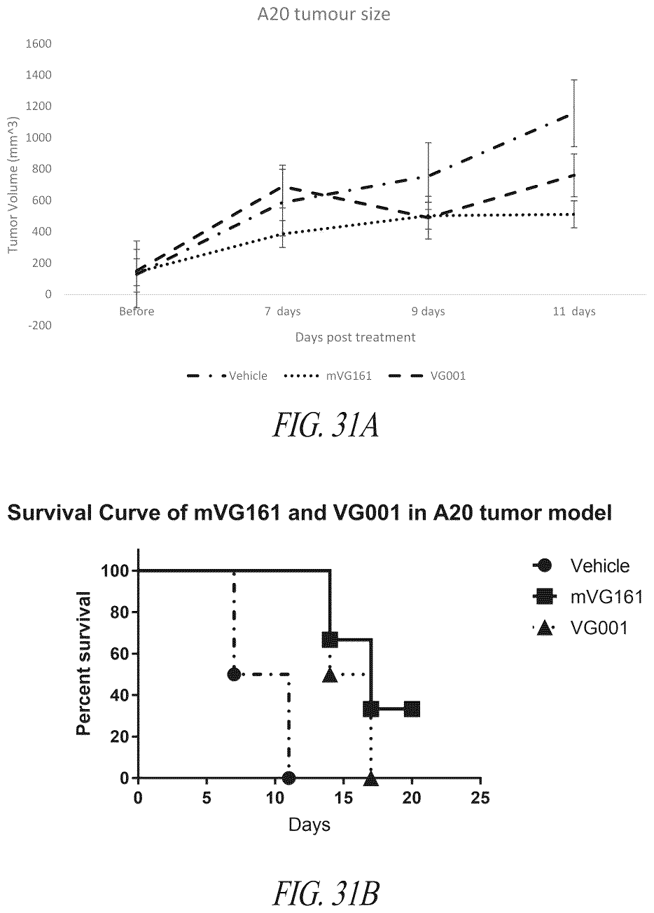

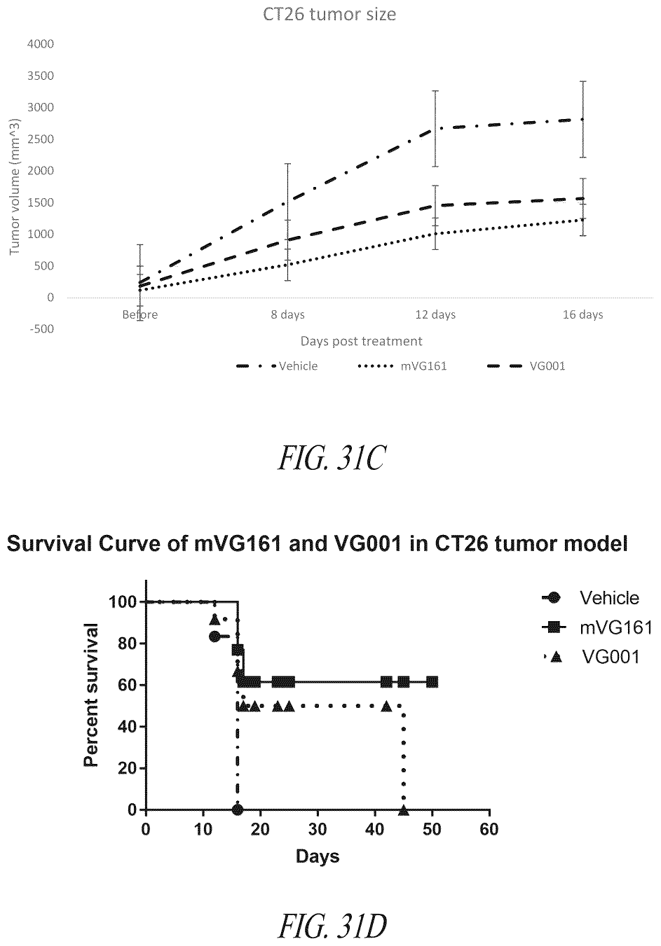

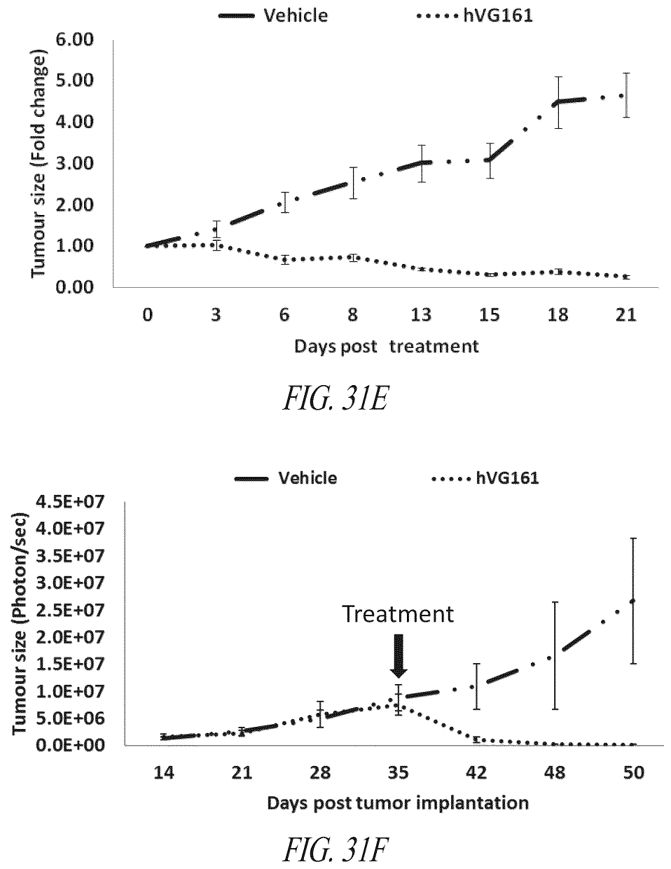

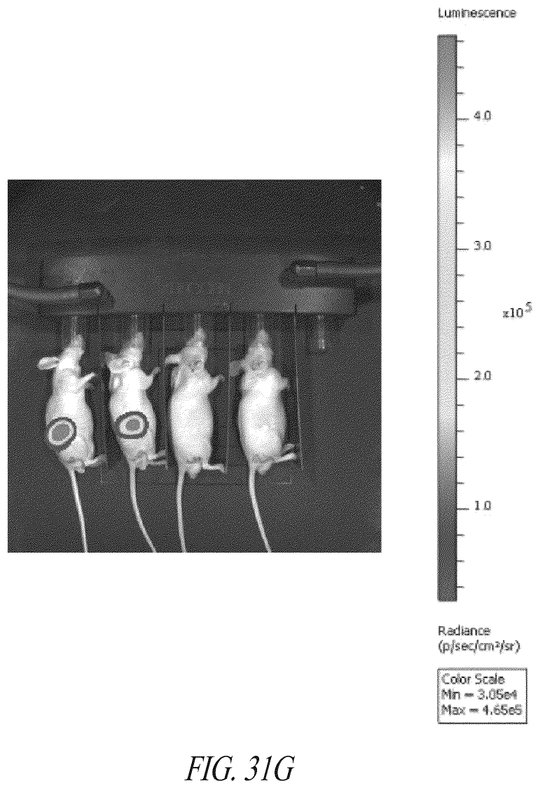

In FIGS. 31A-31B, BALB/c mice bearing A20 murine B-cell lymphoma tumors were injected 5 times intratumorally with a total of 1×10\hat{ }7 PFU/mouse of either VG161-1215PLBm (mVG161) virus or VG001 backbone virus or with PBS (vehicle control). Tumor size measurements were performed at the indicated times post injection. Mice treated with VG161-1215PLBm exhibited a significant (P<0.05) reduction in tumor volume compared to mice treated with PBS.

In FIGS. 31C-31D, BALB/c mice bearing CT26 murine colon carcinoma tumors were injected 5 times intratumorally with a total of 5×10\hat{ }6 PFU/mouse of either VG161-1215PLBm (mVG161) virus or VG001 backbone virus or with PBS (vehicle control). Tumor size measurements were performed at the indicated times post injection. Mice treated with VG161-1215PLBm exhibited a significant (P<0.05) reduction in tumor volume compared to mice treated with PBS.

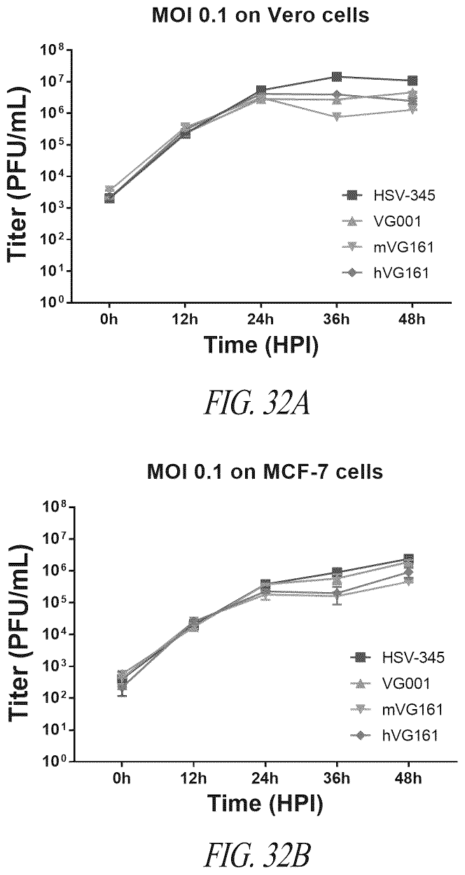

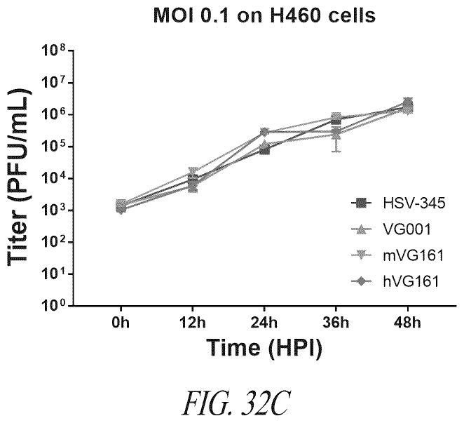

In FIGS. 31E-31G, oHSV treatment of xenograft human prostate tumors in mice was assessed. Twelve mice were implanted with LNCaP human prostate tumor cells in the right lower flank. At 35 days post implantation, a randomly selected group of 6 animals was injected twice intratumorally with a total of 5×10\hat{ }7 PFU/mouse of VG161-1215PLBh (hVG161) virus, while the remaining 6 animals served as a vehicle control and were injected twice with an equivalent volume of PBS. Tumor size measurements were performed using two different methods. Caliper measurements are expressed as fold change in tumor volume at a given time point compared to the tumor volume at the time of virus or PBS injection (FIG. 31E). Tumor-bearing mice treated with VG161-1215PLBh virus exhibited robust tumor shrinkage during the course of the study with over 50% reduction in tumor size at the end of 15 days, while vehicle-treated mice showed approximately 3-fold increases in tumor volume during the same time span. Tumor growth was also monitored using a whole animal bioluminescent imaging system (IVIS Imaging System; Xenogen, Mountain View, CA). Signal intensities were quantified as the sum of all detected photons per second (FIG. 31F). Quantitative imaging of tumor growth using the IVIS system shows an even more dramatic reduction in tumor size in oHSV-treated animals compared to PBS-treated controls, with fluorescence dropping to undetectable levels by 50 days post tumor implantation (FIG. 32G; two vehicle controls on left and two oHSV-treated mice on right).

### Example 19

### Replication of hVG161 in Cell Lines

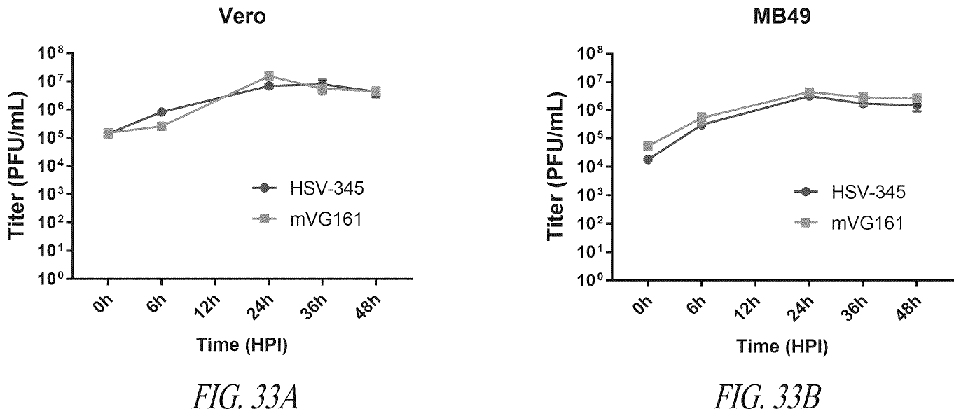

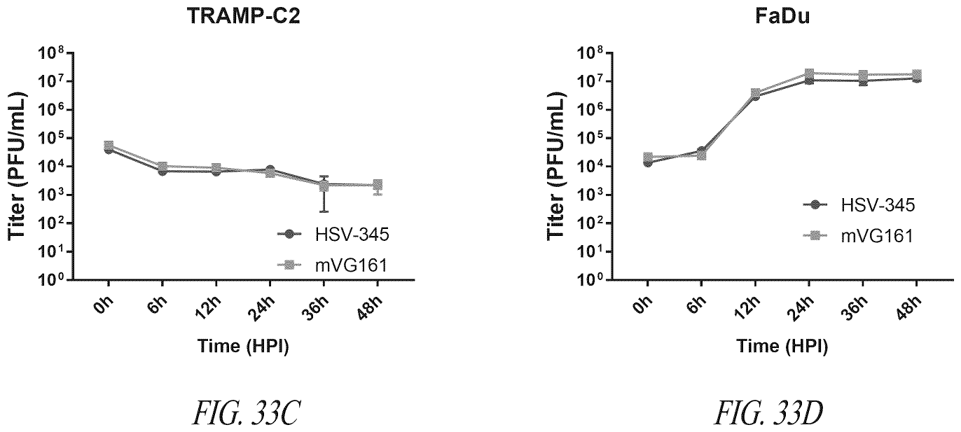

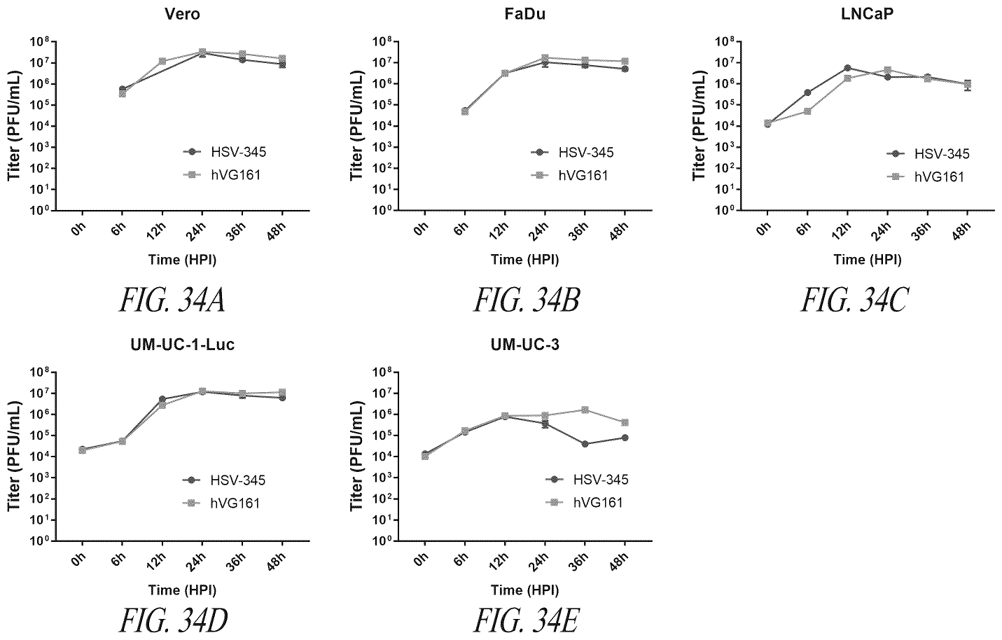

The growth curve and cytotoxicity data in FIGS. 32A-C, FIGS. 33A-D and FIGS. 34A-E show that hVG161 viruses replicate as well as the parental HSV-345 virus. These data also show that the viruses do not grow as well in mouse tumor cell lines compared to human cell lines, but HSV-1 is known to grow poorly in mouse cells.

### Example 20

### Evaluation of Virus Modifications

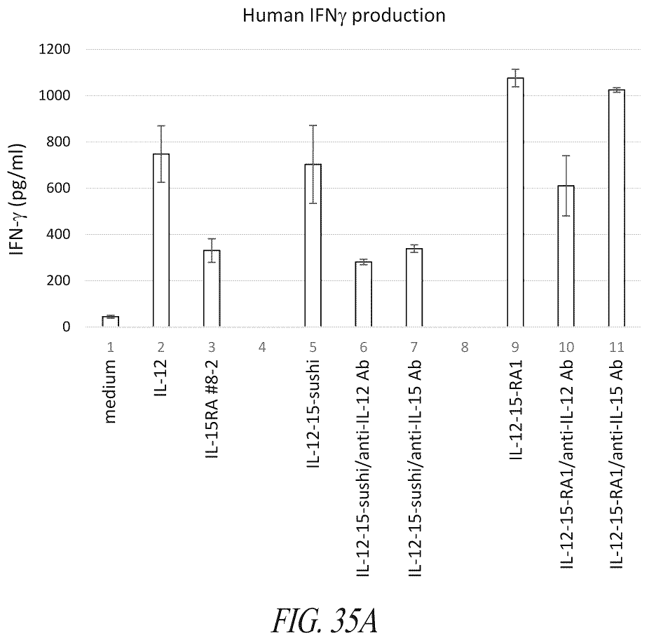

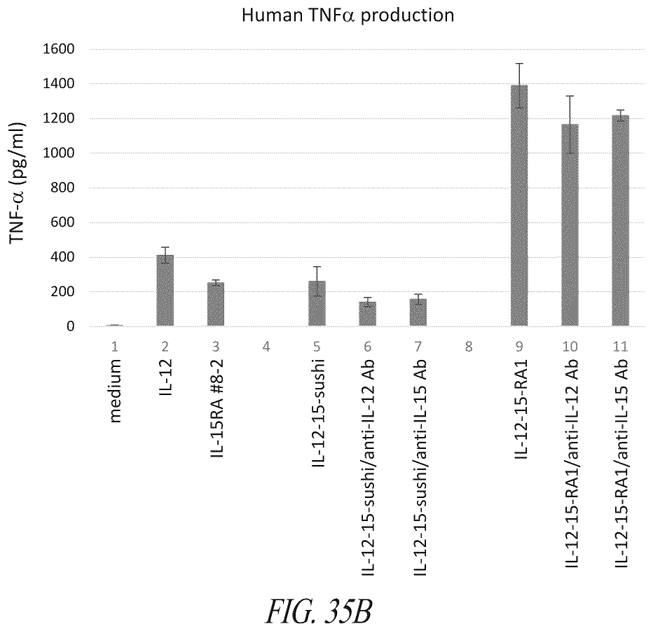

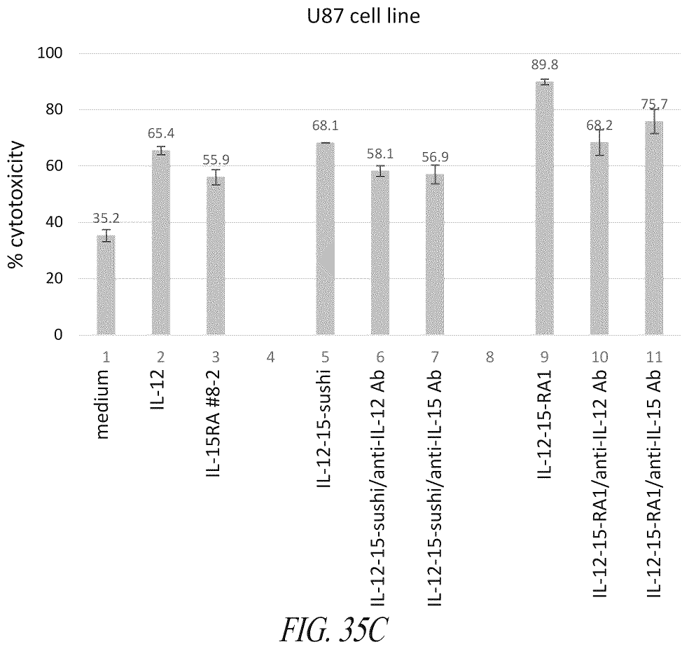

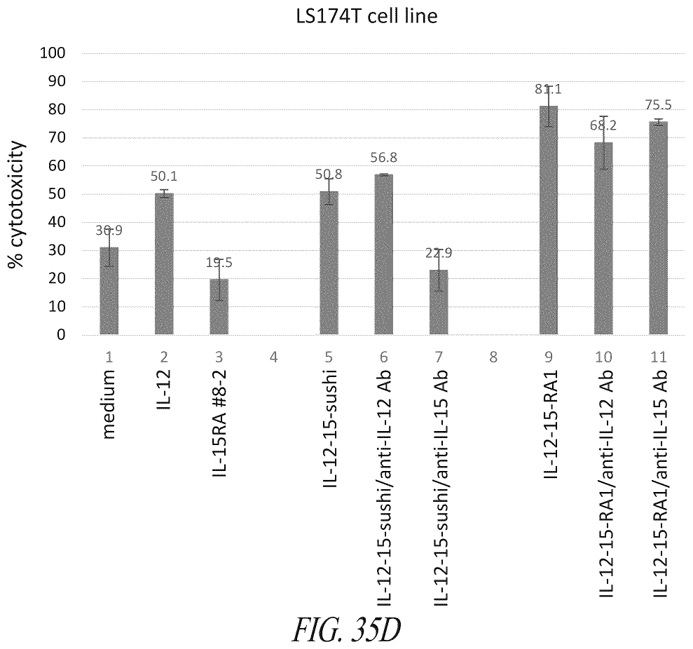

Human PBMCs were stimulated with medium alone, recombinant IL-12 alone, recombinant IL-15 alone, or IL-12 plus different forms of IL-15/IL-15RA1 complex with or without anti-IL-12 (6 mg/ml) or anti-IL-15 (0.5 mg/ml) neutralizing antibody for 48 hours. Cultured supernatants were subsequently harvested for human IFNg and human TNFa production using ELISA assays as shown in FIGS. 35A and 35B.

To assess cytotoxicity against tumour cells, calcein-AM-labelled tumour cells were co-incubated with stimulated human PBMCs for 24 hours. Supernatants were harvested for measurement of released fluorescence. Supernatant harvested from calcein-labelled tumour cells incubated with medium only was used as minimum release, and supernatant harvested from calcein-labelled tumour cells incubated with lysis buffer was used as Maximum release. The percentage of cytotoxicity was calculated based on the formula: [(actual reading−minimum release)/(Maximum release−minimum release)]×100%. Cytotoxicity result for U87 tumour cells is shown as FIG. 35C and MDA-MB-231 tumour cells is shown as FIG. 35D.

### Example 21

### In Vitro Efficacy Data

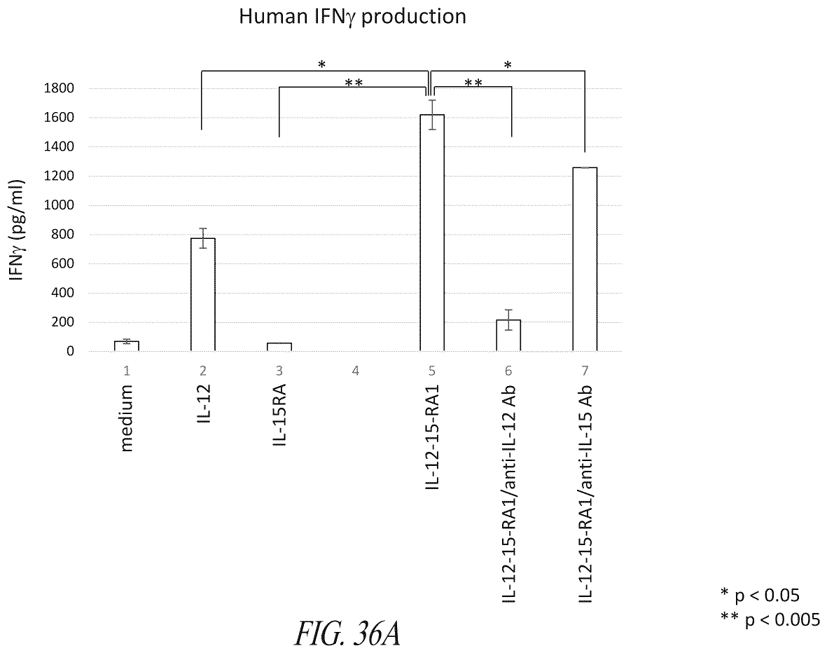

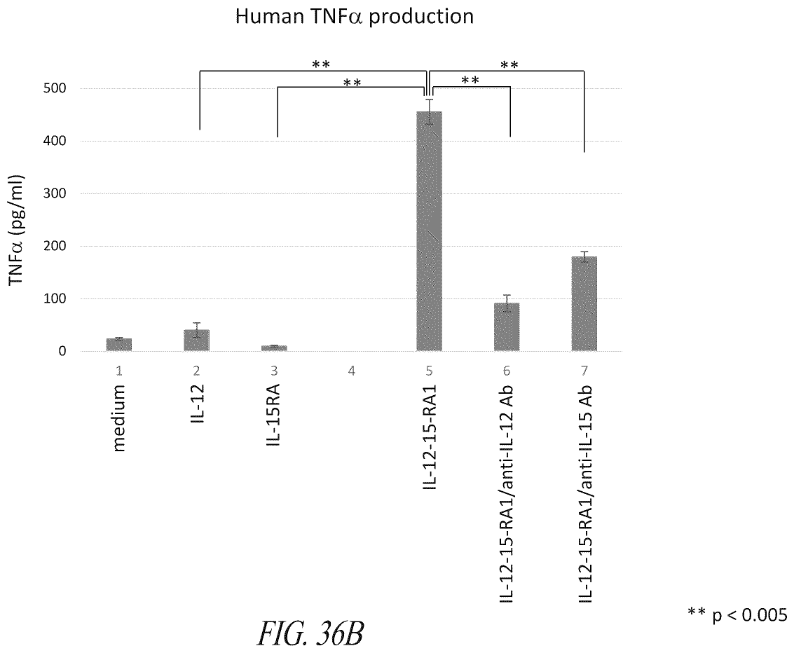

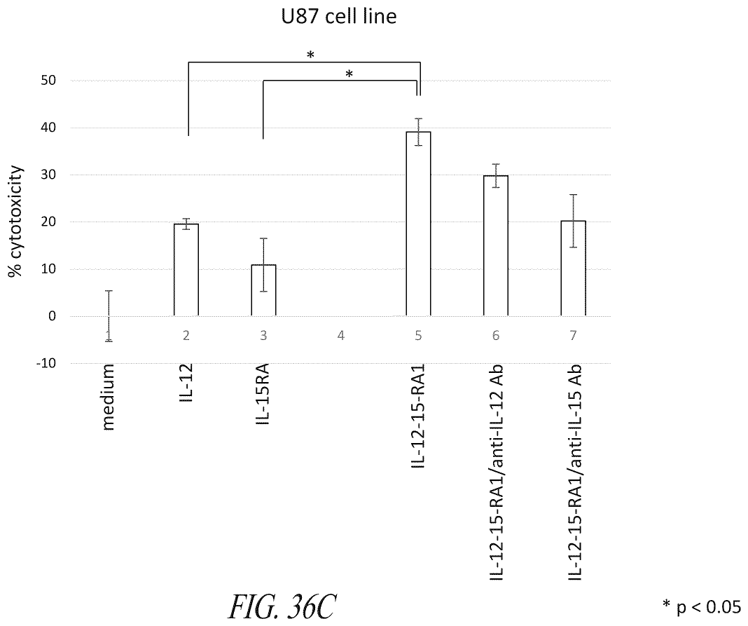

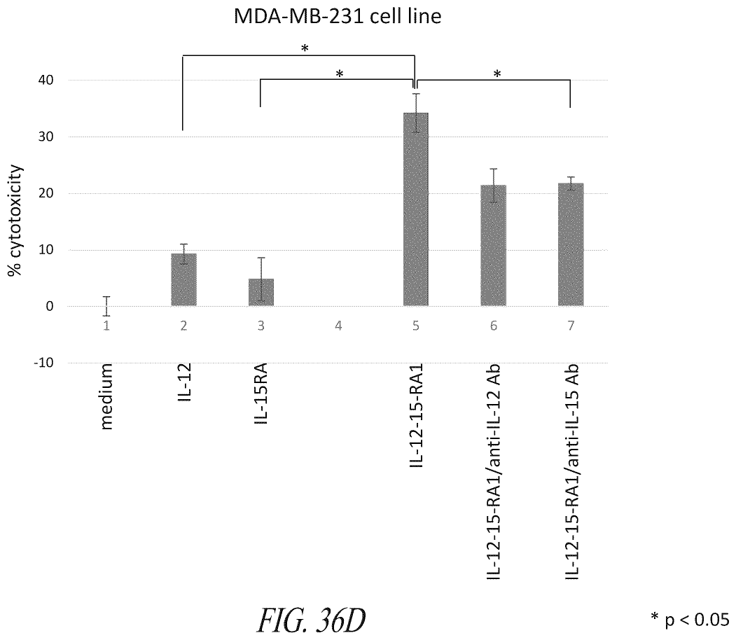

Human peripheral blood mononuclear cells (PBMCs) were stimulated with medium alone, recombinant IL-12 alone, recombinant IL-15 alone, or IL-12 plus IL-15/IL-15RA1 complex with or without anti-IL-12 (6 mg/ml) or anti-IL-15 (0.5 mg/ml) neutralizing antibody for 48 hours. Cultured supernatants were subsequently harvested for human IFNg and human TNFa production using ELISA assays as shown in FIGS. 36A and B.

To assess cytotoxicity against tumour cells, 1×10⁴ calcein-AM-labelled tumour cells were co-incubated with 1×10⁵ stimulated human PBMCs for 24 hours. Supernatants were harvested for measurement of released fluorescence. Supernatant harvested from calcein-labelled tumour cells incubated with medium only was used as minimum release, and supernatant harvested from calcein-labelled tumour cells incubated with lysis buffer was used as Maximum release. The percentage of cytotoxicity was calculated based on the formula: [(actual reading−minimum release)/(Maximum release−minimum release)]×100%. Cytotoxicity results for U87 tumour cells are shown as FIG. 36C and results for MDA-MB-231 tumour cells are shown as FIG. 36D.

### Example 22

### VG161h Infected Tumour Cells Produce Human IL-12, Human IL-15/IL15Ra, and Human IgG4

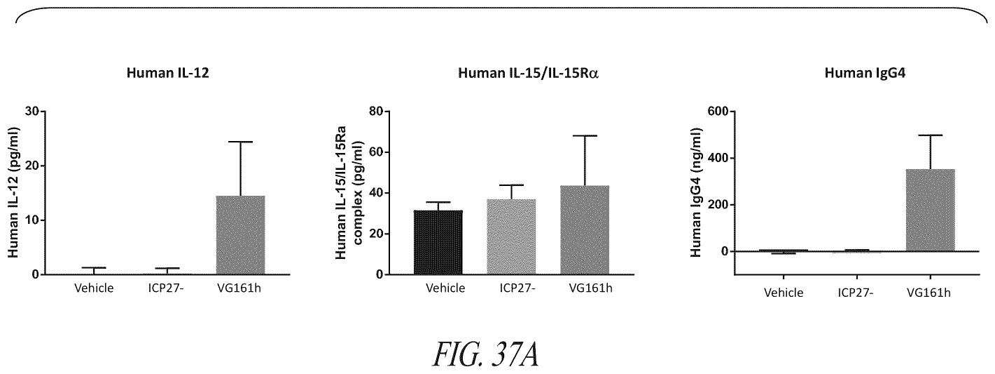

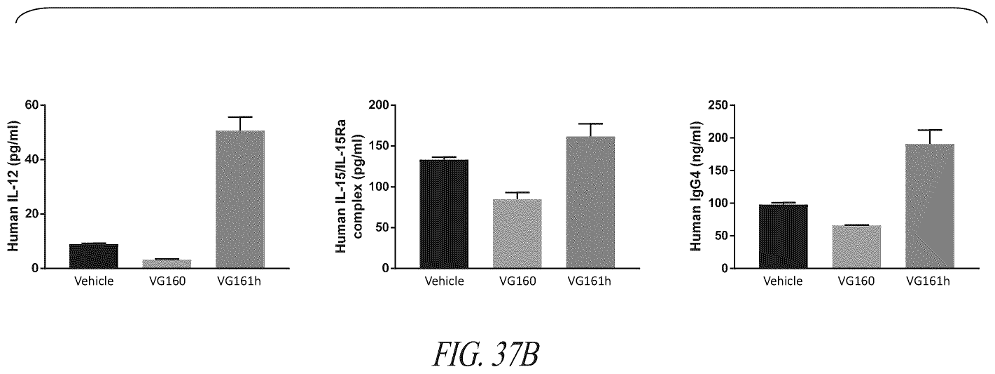

Briefly, LNCaP cells were implanted in nude mice and received injection of vehicle, ICP27-, or VG161h virus. Serum and tumour samples were harvested 120 hours after injection and the productions of human IL-12, human IL-15/IL-15Ra, and human IgG4 were assessed by ELISA. The results are shown in FIG. 37A

Fadu cells were implanted in nude mice and received injection of vehicle, VG160, or VG161h virus. Tumour samples were harvested 24 hours after injection and the productions of human IL-12, human IL-15/IL-15Ra, and human IgG4 were assessed by ELISA. The results are shown in FIG. 37B.

### Example 23

### Effect of VG161m in Immune Response

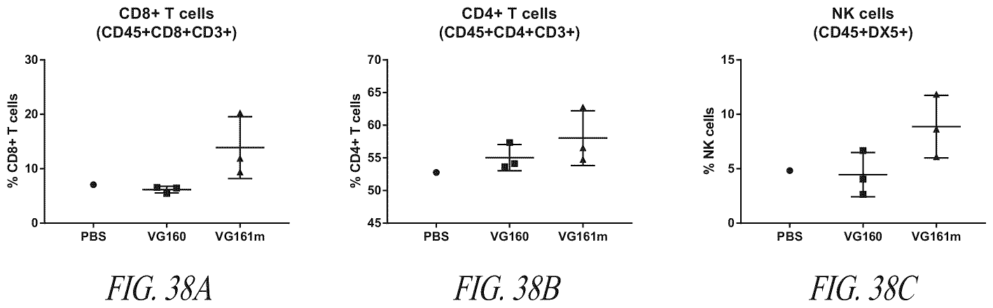

CT26 colon cancer cells were implanted in balb/c mice and received injection of PBS, VG160, or VG161m virus. Tumour samples were harvested 24 hours after injection and the percentage of CD8+ T cells, CD4+ T cells, or NK cells was assessed by flow cytometry. The results are shown in FIGS. 38A-C.

The following are additional exemplary embodiments of the present disclosure:
1) An HSV vector which expresses one or more of IL12, IL15 and/or an ILReceptor 15 alpha subunit. Within one embodiment, the HSV vector comprises an expression cassette which expresses IL12, IL15 and the IL Receptor 15 alpha subunit. Within various embodiments the IL12, IL15 and IL15 Receptor alpha subunit sequences which are expressed are of mammalian origin (e.g., of murine or human origin). Within preferred embodiments the expression cassette expresses murine or human IL12, murine or human IL15, and murine or human IL15Receptor alpha subunit. Within yet other embodiments the expression cassette expresses either murine or human IL12, hIL15, and murine and h15Receptor alpha subunit.
2) The HSV vector of embodiment 1, wherein nucleic acid sequence encoding a self-cleaving 2A peptide is located in-frame between coding sequences for IL12, IL15, and IL15Receptor alpha subunit. Within preferred embodiments, IL12 is a murine or human sequence, IL15 is human sequence, and IL15 Receptor alpha subunit is a human sequence.
3) The HSV vector of embodiment 2, wherein the nucleic acid sequence encodes a self-cleaving 2A peptide selected from the group consisting of VKQTLNFDLLKLAGDVESNPGP (SEQ ID NO: 520), QCTNYALLKLAGDVESNPGP (SEQ ID NO: 521), ATNF-SLLKQAGDVEENPGP (SEQ ID NO: 522), HYAGYFADLLIHDIETNPGP (SEQ ID NO: 523), GIFN-AHYAGYFADLLIHDIETNPGP (SEQ ID NO: 524), KAVRGYHADYYKQRLIHDVEMNPGP (SEQ ID NO: 525), GATNF-SLLKLAGDVELNPGP (SEQ ID NO: 526), EGRGSLLTCGDVEENPGP (SEQ ID NO: 527), AARQMLLLLSGDVETNPGP (SEQ ID NO: 528), FLRKRTOLLMSGDVESNPGP (SEQ ID NO: 529), GSWTDILLLLSGDVETNPGP (SEQ ID NO: 530), TRAEUEDELIRAGIESNPGP (SEQ ID NO: 531), AKFQIDKILISGDVELNPGP (SEQ ID NO: 532), SKFQIDKILISGDIELNPGP (SEQ ID NO: 533), SSIIRTKMLVSGDVEENPGP (SEQ ID NO: 534) and CDAQRQKLLLSGDIEQNPGP (SEQ ID NO: 535).
4) The HSV vector of any one of embodiments 1 to 3, wherein one or more IRES sequences is located between the coding sequences for IL12, IL15, and IL15Receptor alpha subunit. Within preferred embodiments, IL12 is a murine or human sequence, IL15 is human sequence, and IL15 Receptor alpha subunit is a human sequence.
5) The HSV vector of any one of embodiments 1 to 4, where the IL15 and IL15Receptor alpha subunit are co-expressed using a IRES sequence. Within preferred embodiments, IL12 is a murine or human sequence, IL15 is human sequence, and IL15 Receptor alpha subunit is a human sequence.
6) The HSV vector of any one of embodiments 1 to 5, where the IL15 and IL15Receptor alpha subunit are expressed by a bi-directional promoter. Within preferred embodiments, IL12 is a murine or human sequence, IL15 is human sequence, and IL15 Receptor alpha subunit is a human sequence.
7) The HSV vector of embodiment 6, wherein the bi-directional promoter is bi-CMV.
8) The HSV vector of any one of embodiments 1 to 7, wherein each of the IL15 and IL15Receptor alpha subunit is followed by a nucleic acid sequence encoding Lys5 or Glu5. Within preferred embodiments, IL12 is a murine or human sequence, IL15 is human sequence, and IL15 Receptor alpha subunit is a human sequence.
9) The HSV vector of any one of embodiments 1 to 8, wherein the hIL15Receptor alpha subunit is selected from the group consisting of variant 1, variant 2, variant 3 and variant 4.
10) The HSV vector of any one of embodiments 1 to 9, further comprising an expression cassette for one or more PD-L1 blocking peptides, or, wherein said expression cassette expresses one or more PD-L1 blocking peptides.
11) The HSV vector of any one of embodiments 1 to 10, further comprising sequence encoding a peptide linker between multiple PD-L1 blocking peptides.
12) The HSV vector of any one of embodiments 1 to 11, further comprising one or more IRES sequences between multiple PD-L1 blocking peptides.
13) The HSV vector of any one of embodiments 1 to 12, further comprising sequence encoding an Fc domain linked to the 3′-end of the PD-L1 blocking peptide.
14) The HSV vector of any one of embodiments 1 to 13, where the expression cassette is inserted in the either an internal repeat region or the terminal repeat region of HSV genome.
15) The HSV vector of embodiment 10, wherein the sequence encoding a PD-L1 blocking peptide is inserted in between viral genes, such as, for example, the UL3 and UL4 viral genes, the UL50 and UL51 genes, and/or between US1 and US2.
16) The HSV vector of any one of embodiments 1-15, further comprising an NFkB and an OCT4/SOX2 enhancing element in ICP4 or ICP27 regulatory regions.
17) The HSV vector of any one of embodiments 1-16, wherein the ICP34.5 genes are deleted.
18) The HSV vector of any one of embodiments 1 to 17, wherein the expression cassette comprises at least one bidirectional CMV promoter.
19) The HSV vector of any one of embodiments 1 to 18, wherein the expression cassette comprises at least one cellular promoter.
20) The HSV vector of any one of embodiments 1 to 19, the expression cassette for IL12/IL15/IL15Receptor alpha subunit is inserted into either an internal repeat region or the terminal repeat region where the original viral sequence is replaced by the cassette
21) The HSV vector of any of embodiments 1-20, wherein the HSV is either HSV-1 or HSV-2.
22) The HSV vector of any one of embodiments 1 to 21, wherein the ICP34.5 gene is regulated by a 3′UTR containing target sequences of miRNAs that are under-expressed in tumor cells.
23) A pharmaceutical composition, comprising a HSV vector according to any one of embodiments 1 to 22, and a pharmaceutically acceptable carrier.
24) A method of treating cancer, comprising administering to a patient a HSV vector according to any one of embodiments 1 to 22, or a pharmaceutical composition according to embodiment 23.
25) The method according to embodiment 24 wherein said cancer is selected from the group consisting of carcinomas, leukemia's, lymphomas, myelomas and sarcomas.

All patents, publications, scientific articles, web sites, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Applicants reserve the right to physically incorporate into this specification any and all materials and information from any such patents, publications, scientific articles, web sites, electronically available information, and other referenced materials or documents.

The written description portion of this patent includes all claims. Furthermore, all claims, including all original claims as well as all claims from any and all priority documents, are hereby incorporated by reference in their entirety into the written description portion of the specification, and Applicants reserve the right to physically incorporate into the written description or any other portion of the application, any and all such claims. Thus, for example, under no circumstances may the patent be interpreted as allegedly not providing a written description for a claim on the assertion that the precise wording of the claim is not set forth in haec verba in written description portion of the patent.

The claims will be interpreted according to law. However, and notwithstanding the alleged or perceived ease or difficulty of interpreting any claim or portion thereof, under no circumstances may any adjustment or amendment of a claim or any portion thereof during prosecution of the application or applications leading to this patent be interpreted as having forfeited any right to any and all equivalents thereof that do not form a part of the prior art.

All of the features disclosed in this specification may be combined in any combination. Thus, unless expressly stated otherwise, each feature disclosed is only an example of a generic series of equivalent or similar features.

It is to be understood that while the invention has been described in conjunction with the detailed description thereof, the foregoing description is intended to illustrate and not limit the scope of the invention, which is defined by the scope of the appended claims. Thus, from the foregoing, it will be appreciated that, although specific nonlimiting embodiments of the invention have been described herein for the purpose of illustration, various modifications may be made without deviating from the spirit and scope of the invention. Other aspects, advantages, and modifications are within the scope of the following claims and the present invention is not limited except as by the appended claims.

The specific methods and compositions described herein are representative of preferred nonlimiting embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus, for example, in each instance herein, in nonlimiting embodiments or examples of the present invention, the terms “comprising”, “including”, “containing”, etc. are to be read expansively and without limitation. The methods and processes illustratively described herein suitably may be practiced in differing orders of steps, and that they are not necessarily restricted to the orders of steps indicated herein or in the claims.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by various nonlimiting embodiments and/or preferred nonlimiting embodiments and optional features, any and all modifications and variations of the concepts herein disclosed that may be resorted to by those skilled in the art are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

It is also to be understood that as used herein and in the appended claims, the singular forms “a,” “an,” and “the” include plural reference unless the context clearly dictates otherwise, the term “X and/or Y” means “X” or “Y” or both “X” and “Y”, and the letter “s” following a noun designates both the plural and singular forms of that noun. In addition, where features or aspects of the invention are described in terms of Markush groups, it is intended, and those skilled in the art will recognize, that the invention embraces and is also thereby described in terms of any individual member and any subgroup of members of the Markush group, and applicants reserve the right to revise the application or claims to refer specifically to any individual member or any subgroup of members of the Markush group.

Other nonlimiting embodiments are within the following claims. The patent may not be interpreted to be limited to the specific examples or nonlimiting embodiments or methods specifically and/or expressly disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

1. An HSV vector comprising an expression cassette for IL12, IL15, and IL15Receptor alpha subunit.

2. (canceled)

3. (canceled)

4. (canceled)

5. The HSV vector of claim 1, where the IL15 and IL15Receptor alpha subunit are co-expressed using a IRES sequence.

6. (canceled)

7. The HSV vector of claim 1 wherein the bi-directional promoter is bi-CMV.

8. The HSV vector of claim 1, wherein each of the IL15 and IL15Receptor alpha subunit is followed by a nucleic acid sequence encoding Lys5 or Glu5.

9. (canceled)

10. The HSV vector of claim 1, further comprising an expression cassette for one or more PD-L1 blocking peptides.

11. (canceled)

12. The HSV vector of claim 10, further comprising one or more IRES sequences between multiple PD-L1 blocking peptides.

13. (canceled)

14. (canceled)

15. The HSV vector of claim 10, wherein the sequence encoding PD-L1 blocking peptides is inserted in between UL3 and UL4 viral genes.

16. (canceled)

17. (canceled)

18. The HSV vector of claim 1, wherein the expression cassette comprises at least one bidirectional CMV promoter.

19. (canceled)

20. The HSV vector of claim 1, wherein the expression cassette for IL12/IL15/IL15Receptor alpha subunit is inserted into either the internal repeat region or the terminal repeat region where the original viral sequence is replaced by the cassette.

21. (canceled)

22. (canceled)

23. A pharmaceutical composition, comprising a HSV vector according to claim 1, and a pharmaceutically acceptable carrier.

24. (canceled)

25. (canceled)

26. A method of enhancing the efficacy of an immune response, comprising administering a pharmaceutical composition according to claim 23 to a patient having cancer.

27. The method according to claim 26 wherein said cancer is selected from the group consisting of carcinomas, leukemia's, lymphomas, myelomas and sarcomas.

28. The method according to claim 26 wherein said cancer is a melanoma.

29. The method according to claim 26 wherein said cancer is a blastoma.

30. The method according to claim 29 wherein said blastoma is selected from the group consisting of glioblastomas, medulloblastomas and retinoblastomas.

31. The method according to claim 26 wherein said cancer is selected from the group consisting of bile duct cancer, breast, cervix, colorectal, acoustic neuroma, astrocytoma, craniopharyogioma, ependymoma, glioblastoma, hemangioblastoma, menangioma, neuroblastoma, oligodendroglioma, pinealoma, endometrial lining, kidney, larynx, lung, liver, oral cavity, ovaries, pancreas, prostate, squamous cell carcinoma and thyroid.
